SEQUENCE LISTING (30 sequences)

SEQ ID NO: 1 - protein, 424 aa, synthetic construct
  REGION 1..424: note = Description of Artificial Sequence: Synthetic polypeptide
MASSNLIKQLQERGLVAQVTDEEALAERLAQGPIALYCGFDPTADSLHLGHLVPLLCLKRFQQAGHKPVALVGGATGLIGDPSFKAAERKLNTEETVQEWVDKIRKQVAPFLDFDCGENSAIAANNYDWFGNMNVLTFLRDIGKHFSVNQMINKEAVKQRLNREDQGISFTEFSYNLLQGYDFACLNKQYGVVLQIGGSDQWGNITSGIDLTRRLHQNQVFGLTVPLITKADGTKFGKTEGGAVWLDPKKTSPYKFYQFWINTADADVYRFLKFFTFMSIEEINALEEEDKNSGKAPRAQYVLAEQVTRLVHGEEGLQAAKRITECLFSGSLSALSEADFEQLAQDGVPMVEMEKGADLMQALVDSELQPSRGQARKTIASNAITINGEKQSDPEYFFKEEDRLFGRFTLLRRGKKNYCLICWK

SEQ ID NO: 2 - protein, 860 aa, synthetic construct [sequence omitted]

SEQ ID NO: 3 - DNA, 1275 bp, Escherichia coli
  source 1..1275: mol_type = unassigned DNA
atggcaagcagtaacttgattaaacaattgcaagagcgggggctggtagcccaggtgacggacgaggaagcgttagcagagcgactggcgcaaggcccgatcgcgctctattgcggcttcgatcctaccgctgacagcttgcatttggggcatcttgttccattgttatgcctgaaacgcttccagcaggcgggccacaagccggttgcgctggtaggcggcgcgacgggtctgattggcgacccgagcttcaaagctgccgagcgtaagctgaacaccgaagaaactgttcaggagtgggtggacaaaatccgtaagcaggttgccccgttcctcgatttcgactgtggagaaaactctgctatcgcggcgaacaactatgactggttcggcaatatgaatgtgctgaccttcctgcgcgatattggcaaacacttctccgttaaccagatgatcaacaaagaagcggttaagcagcgtctcaaccgtgaagatcaggggatttcgttcactgagttttcctacaacctgttgcagggttatgattttgcctgtttgaacaaacagtacggtgtggtgctgcaaattggtggttctgaccagtggggtaacatcacttctggtatcgacctgacccgtcgtctgcatcagaatcaggtgtttggcctgaccgttccgctgatcactaaagcagatggcaccaaatttggtaaaactgaaggcggcgcagtctggttggatccgaagaaaaccagcccgtacaaattctaccagttctggatcaacactgcggatgccgacgtttaccgcttcctgaagttcttcacctttatgagcattgaagagatcaacgccctggaagaagaagataaaaacagcggtaaagcaccgcgcgcccagtatgtactggcggagcaggtgactcgtctggttcacggtgaagaaggtttacaggcggcaaaacgtattaccgaatgcctgttcagcggttctttgagtgcgctgagtgaagcggacttcgaacagctggcgcaggacggcgtaccgatggttgagatggaaaagggcgcagacctgatgcaggcactggtcgattctgaactgcaaccttcccgtggtcaggcacgtaaaactatcgcctccaatgccatcaccattaacggtgaaaaacagtccgatcctgaatacttctttaaagaagaagatcgtctgtttggtcgttttaccttactgcgtcgcggtaaaaagaattactgtctgatttgctggaaataa

SEQ ID NO: 4 - protein, 860 aa, Escherichia coli [sequence omitted]

SEQ ID NO: 5 - DNA, 2583 bp, Escherichia coli
  source 1..2583: mol_type = unassigned DNA
atgcaagagcaataccgcccggaagagatagaatccaaagtacagcttcattgggatgagaagcgcacatttgaagtaaccgaagacgagagcaaagagaagtattactgcctgtctatgcttccctatccttctggtcgactacacatgggccacgtacgtaactacaccatcggtgacgtgatcgcccgctaccagcgtatgctgggcaaaaacgtcctgcagccgatcggctgggacgcgtttggtctgcctgcggaaggcgcggcggtgaaaaacaacaccgctccggcaccgtggacgtacgacaacatcgcgtatatgaaaaaccagctcaaaatgctgggctttggttatgactggagccgcgagctggcaacctgtacgccggaatactaccgttgggaacagaaattcttcaccgagctgtataaaaaaggcctggtatataagaagacttctgcggtcaactggtgcccgaacgaccagaccgtactggcgaacgaacaagttatcgacggctgctgctggcgctgcgataccaaagttgaacgtaaagagatcccgcagtggtttatcaaaatcactgcttacgctgacgagctgctcaacgatctggataaactggatcactggccagacaccgttaaaaccatgcagcgtaactggatcggtcgttccgaaggcgtggagatcaccttcaacgttaacgactatgacaacacgctgaccgtttacactacccgcccggacacctttatgggttgtacctacctggcggtagctgcgggtcatccgctggcgcagaaagcggcggaaaataatcctgaactggcggcctttattgacgaatgccgtaacaccaaagttgccgaagctgaaatggcgacgatggagaaaaaaggcgtcgatactggctttaaagcggttcacccattaacgggcgaagaaattcccgtttgggcagcaaacttcgtattgatggagtacggcacgggcgcagttatggcggtaccggggcacgaccagcgcgactacgagtttgcctctaaatacggcctgaacatcaaaccggttatcctggcagctgacggctctgagccagatctttctcagcaagccctgactgaaaaaggcgtgctgttcaactctggcgagttcaacggtcttgaccatgaagcggccttcaacgccatcgccgataaactgactgcgatgggcgttggcgagcgtaaagtgaactaccgcctgcgcgactggggtgtttcccgtcagcgttactggggcgcgccgattccgatggtgacgctggaagacggtaccgtaatgccgaccccggacgaccagctgccggtgatcctgccggaagatgtggtaatggacggcattaccagcccgattaaagcagatccggagtgggcgaaaactaccgttaacggtatgccagcactgcgtgaaaccgacactttcgacacctttatggagtcctcctggtactatgcgcgctacacttgcccgcagtacaaagaaggtatgctggattccgaagcggctaactactggctgccggtggatatctacattggtggtattgaacacgccattatgcacctgctctacttccgcttcttccacaaactgatgcgtgatgcaggcatggtgaactctgacgaaccagcgaaacagttgctgtgtcagggtatggtgctggcagatgccttctactatgttggcgaaaacggcgaacgtaactgggtttccccggttgatgctatcgttgaacgtgacgagaaaggccgtatcgtgaaagcgaaagatgcggcaggccatgaactggtttataccggcatgagcaaaatgtccaagtcgaagaacaacggtatcgacccgcaggtgatggttgaacgttacggcgcggacaccgttcgtctgtttatgatgtttgcttctccggctgatatgactctcgaatggcaggaatccggtgtggaaggggctaaccgcttcctgaaacgtgtctggaaactggtttacgagcacacagcaaaaggtgatgttgcggcactgaacgttgatgcgctgactgaaaatcagaaagcgctgcgtcgcgatgtgcataaaacgatcgctaaagtgaccgatgatatcggccgtcgtcagaccttcaacaccgcaattgcggcgattatggagctgatgaacaaactggcgaaagcaccaaccgatggcgagcaggatcgcgctctgatgcaggaagcactgctggccgttgtccgtatgcttaacccgttcaccccgcacatctgcttcacgctgtggcaggaactgaaaggcgaaggcgatatcgacaacgcgccgtggccggttgctgacgaaaaagcgatggtggaagactccacgctggtcgtggtgcaggttaacggtaaagtccgtgccaaaatcaccgttccggtggacgcaacggaagaacaggttcgcgaacgtgctggccaggaacatctggtagcaaaatatcttgatggcgttactgtacgtaaagtgatttacgtaccaggtaaactcctcaatctggtcgttggctaa

SEQ ID NO: 6 - DNA, 60 bp, synthetic construct
  misc_feature 1..60: note = Description of Artificial Sequence: Synthetic primer
  variation 32: note = a, c, t, g, unknown or other
  source 1..60: mol_type = other DNA
gagcgactggcgcaaggcccgatcgcgctcvnktgcggcttcgatcctaccgctgacagc

SEQ ID NO: 7 - DNA, 60 bp, synthetic construct
  misc_feature 1..60: note = Description of Artificial Sequence: Synthetic primer
  source 1..60: mol_type = other DNA
gagcgactggcgcaaggcccgatcgcgctctattgcggcttcgatcctaccgctgacagc

SEQ ID NO: 8 - DNA, 60 bp, synthetic construct
  misc_feature 1..60: note = Description of Artificial Sequence: Synthetic primer
  variation 29: note = a, c, t, g, unknown or other
  source 1..60: mol_type = other DNA
cagcaggcgggccacaagccggttgcgvnkgtaggcggcgcgacgggtctgattggcgac

SEQ ID NO: 9 - DNA, 84 bp, synthetic construct
  misc_feature 1..84: note = Description of Artificial Sequence: Synthetic primer
  variation 31: note = a, c, t, g, unknown or other
  variation 43: note = a, c, t, g, unknown or other
  source 1..84: mol_type = other DNA
cgttcactgagttttcctacaacctgttgvnkggttatrrtvnkgcctgtkyaaacaaacagtacggtgtggtgctgcaaattg

SEQ ID NO: 10 - DNA, 59 bp, synthetic construct
  misc_feature 1..59: note = Description of Artificial Sequence: Synthetic primer
  variation 31: note = a,c, t, g, unknown or other
  source 1..59: mol_type = other DNA
gtgctaacaaacagtacggtgtggrgctgvnkattggtggttctgaccagtggggtaac

SEQ ID NO: 11 - DNA, 60 bp, synthetic construct
  misc_feature 1..60: note = Description of Artificial Sequence: Synthetic primer
  variation 29: note = a, c, t, g, unknown or other
  source 1..60: mol_type = other DNA
gctgtcagcggtaggatcgaagccgcamnbgagcgcgatcgggccttgcgccagtcgctc

SEQ ID NO: 12 - DNA, 60 bp, synthetic construct
  misc_feature 1..60: note = Description of Artificial Sequence: Synthetic primer
  source 1..60: mol_type = other DNA
gctgtcagcggtaggatcgaagccgcaatagagcgcgatcgggccttgcgccagtcgctc

SEQ ID NO: 13 - DNA, 59 bp, synthetic construct
  misc_feature 1..59: note = Description of Artificial Sequence: Synthetic primer
  variation 29: note = a, c, t, g, unknown or other
  source 1..59: mol_type = other DNA
gtcgccaatcagacccgtcgcgccgccmnbtaccgcaaccggctgtggcccgcctgctg

SEQ ID NO: 14 - DNA, 84 bp, synthetic construct
  misc_feature 1..84: note = Description of Artificial Sequence: Synthetic primer
  variation 42: note = a, c, t, g, unknown or other
  variation 54: note = a, c, t, g, unknown or other
  source 1..84: mol_type = other DNA
caatttgcagcaccacaccgtactgtttgtttrmacaggcmnbayyataaccmnbcaacaggttgtaggaaaactcagtgaacg

SEQ ID NO: 15 - DNA, 60 bp, synthetic construct
  misc_feature 1..60: note = Description of Artificial Sequence: Synthetic primer
  variation 30: note = a, c, t, g, unknown or other
  source 1..60: mol_type = other DNA
gttaccccactggtcagaaccaaccaatmnbcagcaccacaccgtactgtttgttagcac

SEQ ID NO: 16 - DNA, 59 bp, synthetic construct
  misc_feature 1..59: note = Description of Artificial Sequence: Synthetic primer
  variation 28: note = a, c, t, g, unknown or other
  variation 31: note = a, c, t, g, unknown or other
  source 1..59: mol_type = other DNA
gcaaagagaagtattactgcctgtctvnkvnkccctatccttctggtcgactacacatg

SEQ ID NO: 17 - DNA, 59 bp, synthetic construct
  misc_feature 1..59: note = Description of Artificial Sequence: Synthetic primer
  source 1..59: mol_type = other DNA
ctgaccgtttacactacccgcccggacgcttttatgggttgtacctacctggcggtagc

SEQ ID NO: 18 - DNA, 73 bp, synthetic construct
  misc_feature 1..73: note = Description of Artificial Sequence: Synthetic primer
  variation 40..41: note = a, c, t, g, unknown or other
  source 1..73: mol_type = other DNA
gaaaccgacactttcgacacctttatggagrsttcctggnnytatgcgcgctacacttgcccgcagtacaaag

SEQ ID NO: 19 - DNA, 60 bp, synthetic construct
  misc_feature 1..60: note = Description of Artificial Sequence: Synthetic primer
  variation 31..32: note = a, c, t, g, unknown or other
  source 1..60: mol_type = other DNA
gcggctaactactggctgccggtggatatcnntattggtggtattgaacacgccattatg

SEQ ID NO: 20 - DNA, 59 bp, synthetic construct
  misc_feature 1..59: note = Description of Artificial Sequence: Synthetic primer
  variation 29..30: note = a, c, t, g, unknown or other
  source 1..59: mol_type = other DNA
tattggtggtattgaacacgccattatgnntctgctctacttccgcttcttccacaaac

SEQ ID NO: 21 - DNA, 59 bp, synthetic construct
  misc_feature 1..59: note = Description of Artificial Sequence: Synthetic primer
  variation 29: note = a,c,t,g, unknown or other
  variation 32: note = a,c,t,g, unknown or other
  source 1..59: mol_type = other DNA
catgtgtagtcgaccagaaggatagggmnbmnbagacaggcagtaatacttctctttgc

SEQ ID NO: 22 - DNA, 59 bp, synthetic construct
  misc_feature 1..59: note = Description of Artificial Sequence: Synthetic primer
  source 1..59: mol_type = other DNA
gctaccgccaggtaggtacaacccataaaagcgtccgggcgggtagtgtaaacggtcag

SEQ ID NO: 23 - DNA, 73 bp, synthetic construct
  misc_feature 1..73: note = Description of Artificial Sequence: Synthetic primer
  variation 33..34: note = a, c, t, g, unknown or other
  source 1..73: mol_type = other DNA
ctttgtactgcgggcaagtgtagcgcgcatarnnccaggaasyctccataaaggtgtcgaaagtgtcggtttc

SEQ ID NO: 24 - DNA, 60 bp, synthetic construct
  misc_feature 1..60: note = Description of Artificial Sequence: Synthetic primer
  variation 29..30: note = a, c, t, g, unknown or other
  source 1..60: mol_type = other DNA
cataatggcgtgttcaataccaccaatanngatatccaccggcagccagtagttagccgc

SEQ ID NO: 25 - DNA, 59 bp, synthetic construct
  misc_feature 1..59: note = Description of Artificial Sequence: Synthetic primer
  variation 30..31: note = a, c, t, g, unknown or other
  source 1..59: mol_type = other DNA
gtttgtggaagaagcggaagtagagcaganncataatggcgtgttcaataccaccaata

SEQ ID NO: 26 - DNA, 73 bp, synthetic construct
  misc_feature 1..73: note = Description of Artificial Sequence: Synthetic primer
  variation 40..41: note = a, c, t, g, unknown or other
  source 1..73: mol_type = other DNA
caaaccgacactttcgacacctttatggagrsttcctggnnytatgcgcgctacacttgcccgcagtacaaag

SEQ ID NO: 27 - DNA, 94 bp, synthetic construct
  misc_feature 1..94: note = Description of Artificial Sequence: Synthetic primer
  variation 32..33: note = a, c, t, g, unknown or other
  variation 62..63: note = a, c, t, g, unknown or other
  source 1..94: mol_type = other DNA
gcggctaactactggctgccggttggatatcnntattggtggtattgaacacgccattatgnntctgctctacttccgcttcttccacaaactg

SEQ ID NO: 28 - DNA, 60 bp, synthetic construct
  misc_feature 1..60: note = Description of Artificial Sequence: Synthetic primer
  source 1..60: mol_type = other DNA
gctaccgccaggtaggtacaacccataaaagcgtccgggcgggtagtagtaaacggtcag

SEQ ID NO: 29 - DNA, 93 bp, synthetic construct
  misc_feature 1..93: note = Description of Artificial Sequence: Synthetic primer
  variation 32..33: note = a, c, t, g, unknown or other
  variation 62..63: note = a, c, t, g, unknown or other
  source 1..93: mol_type = other DNA
cagtttgtggaagaagcggaagtagagcaganncataatggcgtgttcaataccaccaatanngatatccaccggcagccagtagttagccgc

SEQ ID NO: 30 - protein, 424 aa, Escherichia coli; identical to SEQ ID NO: 1

(19) United States
(12) Patent Application Publication
(10) Pub. No.: US 2024/0200050 A1
(43) Pub. Date: Jun. 20, 2024

(54) AMINOACYL TRANSFER RNA SYNTHETASES

(71) Applicant: Trustees of Tufts College, Medford, MA (US)

(72) Inventors: James A. VAN DEVENTER, Medford, MA (US); Jessica T. STIEGLITZ, Medford, MA (US)

(73) Assignee: Trustees of Tufts College, Medford, MA (US)

(21) Appl. No.: 18/513,092

(22) Filed: Nov. 17, 2023

Related U.S. Application Data
(63) Continuation of application No. PCT/US2022/029775, filed on May 18, 2022.
(60) Provisional application No. 63/190,336, filed on May 19, 2021.

(51) Int. Cl. C12N 9/00; C12N 15/52; C12N 15/66

(52) U.S. Cl. CPC C12N 9/93; C12N 15/52; C12N 15/66; C12Y 601/01001; C12Y 601/01004

(57) ABSTRACT

The invention provides aminoacyl-tRNA synthetases, compositions thereof, and methods for use thereof.

### CROSS-REFERENCE TO RELATED APPLICATIONS

This application is a continuation under 35 U.S.C. § 111(a) of PCT International Patent Application No. PCT/US2022/029775, filed May 18, 2022, designating the United States and published in English, which claims priority to and the benefit of U.S. Provisional Application No. 63/190,336, filed May 19, 2021, the entire contents of each of which are incorporated by reference herein.

### STATEMENT OF RIGHTS TO INVENTIONS MADE UNDER FEDERALLY SPONSORED RESEARCH

This invention was made with government support under Grant No. W911NF-16-1-0175 from the Department of Defense; Grant No. 2016231237 from the National Science Foundation; and Grant No. R35GM133471 from the National Institute of Health. The government has certain rights in the invention.

### SEQUENCE LISTING

The present application contains a Sequence Listing which has been submitted electronically in XML format and is herein incorporated by reference in its entirety. The Sequence Listing XML file, created on Feb. 7, 2024, is named 167774-012502US-Sequence_Listing.xml and is 55,383 bytes in size.

### BACKGROUND OF THE INVENTION

The ability to produce chemically versatile proteins with encoded noncanonical amino acids (ncAAs) at specific sites supports a growing number of applications in chemical and synthetic biology. Aminoacyl-tRNA synthetases (aaRSs) evolved to maintain the fidelity of genetic code translation—that is, to precisely charge canonical amino acids (cAAs) to their cognate tRNAs while discriminating against other potential substrates. Aminoacyl-tRNA synthetase (aaRS) characteristics such as solubility and specificity are known to play key roles for individual applications, but it is not yet clear how best to evolve aminoacyl-tRNA synthetases (aaRSs) to address those needs. Thus, there is a need for aminoacyl-tRNA synthetases with improved characteristics.

### SUMMARY OF THE INVENTION

As described below, the invention of the disclosure features aminoacyl-tRNA synthetases, compositions thereof, and methods for use thereof.

In one aspect, the invention of the disclosure features a Tyrosyl-tRNA Synthetase (TyrRS) polypeptide, or a functional fragment thereof, having at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1). The polypeptide, contains an alteration at an amino acid position selected from any one or more of Y37, L71, V72, Q179, D182, and Q195. The polypeptide, has tRNA synthetase activity.

In one aspect, the invention of the disclosure features in vitro method of producing a protein containing a noncanonical amino acid. The method involves (a) contacting an in vitro translation system with a Tyrosyl-tRNA Synthetase (TyrRS) polypeptide, or a functional fragment thereof, having at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and containing an alteration at an amino acid position selected from one or more of Y37, L71, V72, Q179, D182, and Q195 and having tRNA synthetase activity. The method further involves (b) contacting the in vitro translation system of (a) with one or more noncanonical amino acids or a composition thereof and a polynucleotide encoding a protein containing one or more noncanonical amino acids. The method also involves (c) expressing the protein containing one or more noncanonical amino acids using the in vitro translation system.

In another aspect, the invention of the disclosure features a method of producing a protein containing a noncanonical amino acid. The method involves (a) contacting a cell with an expression vector encoding a Tyrosyl-tRNA Synthetase (TyrRS) polypeptide, or a functional fragment thereof The polypeptide has at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1). The polypeptide contains an alteration at an amino acid position selected from any one or more of Y37, L71, V72, Q179, D182, and Q195. The polypeptide has tRNA synthetase activity. The method further involves expressing the polypeptide(s) in the cell. The method involves (b) contacting the cell of (a) with one or more noncanonical amino acids or a composition thereof and a polynucleotide encoding a protein containing one or more noncanonical amino acids. The method also involves (c) expressing the protein containing one or more noncanonical amino acids in the cell and/or on the surface of the cell and/or secreted from the cell.

In another aspect, the invention of the disclosure features a system for producing and selecting for a protein containing a noncanonical amino acid. The system contains an in vitro translation system containing a Tyrosyl-tRNA Synthetase (TyrRS) polypeptide, or a functional fragment thereof, having at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and containing an alteration at an amino acid position selected from one or more of Y37, L71, V72, Q179, D182, and Q195 and having tRNA synthetase activity. The in vitro translation system contains one or more noncanonical amino acids or a composition thereof. A protein containing one or more noncanonical amino acids is produced in the in vitro translation system.

In another aspect, the invention of the disclosure features a system for producing and selecting for a protein containing a noncanonical amino acid. The system contains (a) a cell expressing a Tyrosyl-tRNA Synthetase (TyrRS) polypeptide, or a functional fragment thereof. The polypeptide has at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1). The polypeptide contains an alteration at an amino acid position selected from any one or more of Y37, L71, V72, Q179, D182, and Q195. The polypeptide has tRNA synthetase activity. The cell is contacted with one or more noncanonical amino acids or a composition thereof. A protein containing one or more noncanonical amino acids is produced in the cell and/or on the surface or the cell. The system also contains (b) materials and/or equipment to select for the protein containing one or more noncanonical amino acids produced in the cell and/or on the surface of the cell.

In another aspect, the invention of the disclosure features a method of controlling the replication of a cell or virus. The method involves (a) (i) altering a gene encoding a polypeptide in the cell or a virus infecting the cell to encode the polypeptide altered to contain a noncanonical amino acid and/or (ii) knocking out the gene in the cell or the virus infecting the cell and contacting the cell with a polynucleotide sequence encoding the polypeptide altered to contain the noncanonical amino acid, such that replication of the cell or virus is reduced or eliminated in the absence of expression of the polypeptide containing the noncanonical amino acid. The method further involves (b) contacting the cell with an expression vector encoding a Tyrosyl-tRNA Synthetase (TyrRS) polypeptide, or a functional fragment thereof where the polypeptide has at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and containing an alteration at an amino acid position selected from one or more of Y37, L71, V72, Q179, D182, and Q195 and having tRNA synthetase activity, and expressing the polypeptide(s) in the cell. The method also involves (c) controlling replication of the cell and/or virus by contacting the cell of (a) with the noncanonical amino acid. Replication of the cell and/or virus is reduced or eliminated in the absence of the noncanonical amino acid.

In any of the above aspects, or embodiments thereof, the alteration at Y37 is Y37A, Y37D, Y37E, Y37G, Y37H, Y37I, Y37L, Y37M, Y37Q, Y37T, or Y37V. In any of the above aspects, the alteration at L71 is L71V, L71D, L71I, L71M, L71R, L71T, or L71V. In any of the above aspects, the alteration at V72 is V72A, V72H, V72I, V72L, V72M, V72Q, V72R, V72S, or V72T. In any of the above aspects, the alteration at Q179 is Q179A, Q179D, Q179E, Q179G, Q179H, Q179L, Q179M, Q179N, Q179P, Q179S, Q179T, or Q179V. In any of the above aspects, the alteration at D182 is D182G, or D182S. In any of the above aspects, the polypeptide contains an alteration at F183 selected from F183A, F183D, F183G, F183H, F183I, F183L, F183N, F183P, F183Q, F183R, F183T, and F183V. In any of the above aspects, the polypeptide contains an alteration at L186 selected from L186E, L186S, and L186V. In any of the above aspects, the alteration at Q195 is Q195A, Q195E, Q195G, Q195H, Q195I, Q195K, Q195L, Q195M, Q195S, Q195T, or Q195V. In any of the above aspects, the polypeptide contains a combination of alterations selected from (1) Y37L and L71V, (2) Y37G, L71T, and D182S, (3) L71V and L186A, (4) Y37V and L71V, (5) Y37L, L71V, and L186A, and (6) L71I, V72V, Q179Q, D182G, F183M, L186A, and Q195Q.

In another aspect, the invention of the disclosure features a Leucyl-tRNA Synthetase (LeuRS) polypeptide, or a functional fragment thereof, having at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2). The polypeptide contains an alteration at an amino acid position selected from any one or more of M40, L41, 5496, Y499 and Y527. The polypeptide has tRNA synthetase activity.

In another aspect, the invention of the disclosure provides an in vitro method of producing a protein containing a noncanonical amino acid. The method involve (a) contacting an in vitro translation system with an expression vector encoding a Leucyl-tRNA Synthetase (LeuRS) polypeptide, or a functional fragment thereof, having at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and containing an alteration at an amino acid position selected from one or more of M40, L41, S496, Y499 and Y527 and having tRNA synthetase activity, and expressing the polypeptide(s) in the cell. The method further involves (b) contacting the in vitro translation system of (a) with one or more noncanonical amino acids or a composition thereof and producing a protein containing one or more noncanonical amino acids in the cell. The method also involves (c) expressing the protein containing one or more noncanonical amino acids using the in vitro translation system.

In another aspect, the invention of the disclosure features a method of producing a protein containing a noncanonical amino acid. The method involves (a) contacting a cell with an expression vector. The expression vector encodes a Leucyl-tRNA Synthetase (LeuRS) polypeptide, or a functional fragment thereof. The polypeptide has at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2). The polypeptide contains an alteration at an amino acid position selected from any one or more of M40, L41, S496, Y499 and Y527. The polypeptide has tRNA synthetase activity. The method further involves expressing the polypeptide(s) in the cell. The method also involves (b) contacting the cell of (a) with one or more noncanonical amino acids or a composition thereof and a polynucleotide encoding a protein containing one or more noncanonical amino acids. The method also involves (c) expressing the protein containing one or more noncanonical amino acids in the cell and/or on the surface of the cell and/or secreted from the cell.

In another aspect, the invention of the disclosure features a system for producing and selecting for a protein containing a noncanonical amino acid. The system contains an in vitro translation system containing a Leucyl-tRNA Synthetase (LeuRS) polypeptide, or a functional fragment thereof, having at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and containing an alteration at an amino acid position selected from one or more of M40, L41, S496, Y499 and Y527 and having tRNA synthetase activity. The in vitro translation system contains one or more noncanonical amino acids or a composition thereof. A protein containing one or more noncanonical amino acids is produced in the in vitro translation system.

In another aspect, the invention of the disclosure features a system for producing and selecting for a protein containing a noncanonical amino acid. The system contains (a) a cell expressing a Leucyl-tRNA Synthetase (LeuRS) polypeptide, or a functional fragment thereof.

The polypeptide has at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2). The polypeptide contains an alteration at an amino acid position selected from any one or more of M40, L41, S496, Y499 and Y527. The polypeptide has tRNA synthetase activity. The cell is contacted with one or more noncanonical amino acids or a composition thereof. A protein containing one or more noncanonical amino acids is produced in the cell and/or on the surface or the cell. The system further contains (b) materials and/or equipment to select for the protein containing one or more noncanonical amino acids produced in the cell and/or on the surface of the cell.

In another aspect, the invention of the disclosure features a method of controlling the replication of a cell or virus. The method involves (a)(i) altering a gene encoding a polypeptide in the cell or a virus infecting the cell to encode the polypeptide altered to contain a noncanonical amino acid and/or (ii) knocking out the gene in the cell or the virus infecting the cell, and contacting the cell with a polynucleotide sequence encoding the polypeptide altered to comprise the noncanonical amino acid, such that replication of the cell or virus is reduced or eliminated in the absence of expression of the polypeptide containing the noncanonical amino acid. The method also involves (b) contacting the cell an expression vector encoding a Leucyl-tRNA Synthetase (LeuRS) polypeptide, or a functional fragment thereof, where the polypeptide has at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and containing an alteration at an amino acid position selected from one or more of M40, L41, S496, Y499 and Y527 and having tRNA synthetase activity, and expressing the polypeptide(s) in the cell. The method further involves (c) controlling replication of the cell and/or virus by contacting the cell of (a) with the noncanonical amino acid, where replication of the cell and/or virus is reduced or eliminated in the absence of the noncanonical amino acid.

In any of the above aspects, or embodiments thereof, the alteration at M40 is M40A, M40G, M40L, M40P, M40Q, or M40S. In any of the above aspects, the alteration at L41 is L41A, L41E, L41G, L41H, L41N, L41P, L41T, or L41V. In any of the above aspects, the alteration at S496 is S496A, S496G, or S496T. In any of the above aspects, the alteration at Y499 is Y499A, Y499C, Y499F, Y499G, Y499H, Y499I, Y499L, Y499N, Y499S, Y499T, or Y499V. In any of the above aspects, the alteration at Y527 is Y527C, Y527D, Y527F, Y527G, Y527H, Y527I, Y527N, Y527R, Y527S, Y527T, or Y527V. In any of the above aspects, the polypeptide further contains an alteration at H537 selected from any one or more of H537A, H537C, H537F, H537L, and H537S. In any of the above aspects, the polypeptide contains a combination of alterations selected from (1) M40G and S496T, (2) S496T and H537G, (3) S496G and H537G, (4) L41P, S496G, and H537G, (5) M40P, S496G, H537G, (6) L41G and H537G, (7) M40A and H537G, and (8) M40G, L41P, S496G Y499A, Y527C, and H537G. In another aspect, the invention of the disclosure features a polynucleotide encoding the polypeptide of any one of the above aspects. In another aspect, the invention of the disclosure features an expression vector containing the polynucleotide. In another aspect, the invention of the disclosure features a cell containing the expression vector.

In another aspect, the invention of the disclosure features a protein containing one or more noncanonical amino acids produced, generated or selected by the method or system of any of the above aspects.

In any of the above aspects, or embodiments thereof, the protein possesses one or more improved properties or activities relative to a reference protein lacking one or more of the noncanonical amino acids

In any of the above aspects, or embodiments thereof, the polypeptide has aminoacylation activity polyspecific for at least two noncanonical amino acids.

In any of the above aspects, or embodiments thereof, the polypeptide encodes a noncanonical amino acid with a relative readthrough efficiency of at least 0.01. In any of the above aspects, the polypeptide encodes a noncanonical amino acid with a maximum misincorporation efficiency of less than 0.5.

In any of the above aspects, or embodiments thereof, the noncanonical amino acid(s) is selected from one or more of O-methyl-L-tyrosine (OmeY); p-acetyl-L-phenylalanine (AcF); p-azido-L-phenylalanine (AzF); p-propargyloxy-L-phenylalanine (OPG); 4-azidomethyl-L-phenylalanine (AzMF); 4-borono-L-phenylalanine (BPhe); 3,4-dihydroxy-L-phenylalanine (DOPA); 4-iodo-L-phenylalanine (IPhe); L-α-aminocaprylic acid (AC); NE-azido-L-lysine (AzK); 3-Amino-L-tyrosine (ATyr); 4-Amino-L-phenylalanine (APhe); dimethyl-L-lysine (DMK); Boc-L-lysine (BocK); (S)-2-amino-6-((2-azidoethoxy)carbonylamino)hexanoic acid (LysN3); and 2-Amino-6-(prop-2-ynoxycarbonylamino)hexanoic acid (LysAlk). In any of the above aspects, or embodiments thereof, the noncanonical amino acid(s) is selected from one or more of O-methyl-L-tyrosine (OmeY); p-acetyl-L-phenylalanine (AcF); p-azido-L-phenylalanine (AzF); p-propargyloxy-L-phenylalanine (OPG); 4-azidomethyl-L-phenylalanine (AzMF); 4-borono-L-phenylalanine (BPhe); 3,4-dihydroxy-L-phenylalanine (DOPA); O-(2-Bromoethyl)-tyrosine (Obey); 4-iodo-L-phenylalanine (IPhe); L-α-aminocaprylic acid (AC); NE-azido-L-lysine (AzK); 3-Amino-L-tyrosine (ATyr); 4-Amino-L-phenylalanine (APhe); dimethyl-L-lysine (DMK); Boc-L-lysine (BocK); (S)-2-amino-6-((2-azidoethoxy)carbonylamino)hexanoic acid (LysN3); and 2-Amino-6-(prop-2-ynoxycarbonylamino)hexanoic acid (LysAlk), 0-(2-Bromoethyl)-L-tyrosine, O-Sulfo-L-tyrosine (SY), 2-amino-3-[4-(carboxymethyl) phenyl]propanoic acid (CMF), L-p-hydroxy-phenyllactic acid (Ester), L-2-Amino-4-phosphonobutyric acid (PSA), O-phospho-L-serine (OPS), Acetyl-L-lysine (AcK), 4-benzoyl-1-phenylalanine (Bpa), and N⁶-((2-(3-methyl-3H-diazirin-3-yl)ethoxy)carbonyl)-L-lysine (Photo-Lysine, Phk). In any of the above embodiments and/or aspects, the noncanonical amino acid is selected from at least one of DOPA and BPhe. In any of the above embodiments and/or aspects, the noncanonical amino acid is selected from at least one of DOPA, BPhe, Obey, and Phk.

In any of the above aspects, or embodiments thereof, the method or system further involves selecting and/or isolating the protein containing one or more noncanonical amino acids from the cell. In any of the above aspects, or embodiments thereof, the protein containing one or more canonical amino acids is displayed on the surface of a yeast cell.

In any of the above aspects, or embodiments thereof, the cell is a mammalian cell, a yeast cell, or a prokaryotic cell. In any of the above aspects, or embodiments thereof, the cell is a mammalian cell, yeast cell, prokaryotic cell, plant cell, or insect cell. In embodiments, the prokaryotic cell is a bacterial cell. In any of the above aspects, or embodiments thereof, the cell is a mammalian cell selected from COS7, CHO, 293T, Hela or Vero cells. In any of the above aspects, or embodiments thereof, the cell is a yeast cell selected from Saccharornyces cerevisiae, Pichia pastoris, Hansenua polymorpha, Yarrowia lipolytica, Arxula adeninivorans, Kluyveronyces lactis, Candida boidinii and Schizosaccharomyces pombe yeast cells. In any of the above aspects, or embodiments thereof, the cell is genetically engineered or mutated to utilize noncanonical amino acids.

In any of the above aspects, or embodiments thereof, the protein is selected from a growth factor, an antibody or a fragment thereof, a cytokine, a chemokine, an extracellular matrix protein, a polypeptide having an immune-modulatory function, an interleukin, an interferon, an immune-checkpoint blockade polypeptide, an antigen recognition polypeptide, a binding agent, and an alpha-helical peptide or ligand thereof. In any of the above aspects, or embodiments thereof, the protein is selected from an immunoglobulin, a single chain antibody, an scFv, a single-domain antibody (nanobody), a fibronectin, a sso7d, a protein containing an alternative binding scaffold, a cytokine, an interleukin, an interferon, insulin, alpha-1 antitrypsin, angiostatin, antihemolytic factor, apolipoprotein, apoprotein, atrial natriuretic factor, atrial natriuretic polypeptide, atrial peptides, C-X-C chemokines, calcitonin, a CC chemokine, CD40 ligand, C-kit Ligand, collagen, colony stimulating factor (CSF), complement factor 5a, complement inhibitor, complement receptor 1, epidermal growth factor (EGF), Eerythropoietin, exfoliating toxins A and B, factor IX, factor VII, factor VIII, factor X, fibroblast frowth factor (FGF), fibrinogen, G-CSF, GM-CSF, glucocerebrosidase, gonadotropin, a hedgehog protein, hemoglobin, hepatocyte growth factor (HGF), hirudin, human serum albumin, insulin-like growth factor (IGF), keratinocyte growth factor (KGF), lactoferrin, leukemia inhibitory factor, luciferase, neurturin, neutrophil inhibitory factor (NIF), oncostatin M, osteogenic protein, parathyroid hormone, PD-ECSF, PDGF, peptide hormones, pleiotropin, protein A, protein G, pyrogenic exotoxins A, B, and C, relaxin, renin, SCF, soluble complement receptor I, soluble I-CAM 1, soluble interleukin receptors, soluble TNF receptor, somatomedin, somatostatin, somatotropin, streptokinase, superantigens, superoxide dismutase (SOD), toxic shock syndrome toxin (TSST-1), thymosin alpha 1, tissue plasminogen activator, tumor necrosis factor beta (TNF beta), tumor necrosis factor receptor (TNFR), tumor necrosis factor-alpha (TNF alpha), vascular endothelial growth factor (VEGF), and urokinase.

In any of the above aspects, or embodiments thereof, a library of proteins containing one or more noncanonical amino acids is expressed in and/or on the surface of the cell.

In any of the above aspects, or embodiments thereof, a protein containing one or more noncanonical amino acids is selected and/or isolated using high throughput screening.

The invention provides aminoacyl-tRNA synthetases, compositions thereof, and methods for use thereof. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “wild type (WT) tyrosyl-tRNA synthetase (tyrRS) polypeptide sequence” is meant a polypeptide or fragment thereof having at least about 85% amino acid identity to GenBank Accession No. QPN93601.1 and having activities that include charging a tRNA molecule with an amino acid. In some embodiments, the amino acid is tyrosine. A representative WT tyrRS polypeptide sequence is provided below:

(SEQ ID NO: 30)
MASSNLIKQLQERGLVAQVTDEEALAERLAQGPIALYCGFDPTADSLHLG
HLVPLLCLKRFQQAGHKPVALVGGATGLIGDPSFKAAERKLNTEETVQEW
VDKIRKQVAPFLDFDCGENSAIAANNYDWFGNMNVLTFLRDIGKHFSVNQ
MINKEAVKQRLNREDQGISFTEFSYNLLQGYDFACLNKQYGVVLQIGGSD
QWGNITSGIDLTRRLHQNQVFGLTVPLITKADGTKFGKTEGGAVWLDPKK
TSPYKFYQFWINTADADVYRFLKFFTFMSIEEINALEEEDKNSGKAPRAQ
YVLAEQVTRLVHGEEGLQAAKRITECLFSGSLSALSEADFEQLAQDGVPM
VEMEKGADLMQALVDSELQPSRGQARKTIASNAITINGEKQSDPEYFFKE
EDRLFGRFTLLRRGKKNYCLICWK.

By “wild type (WT) tyrosyl-tRNA synthetase (tyrRS) polynucleotide sequence” is meant a polynucleotide or fragment thereof having at least about 85% amino acid identity to a nucleotide sequence corresponding to base pairs 2305374 to 2306648 of GenBank Accession No. CP058342.1 and encoding a polypeptide having activities that include charging a tRNA molecule with an amino acid. In some embodiments, the amino acid is tyrosine. A representative WT tyrRS polynucleotide sequence is provided below:

(SEQ ID NO: 3)
ATGGCAAGCAGTAACTTGATTAAACAATTGCAAGAGCGGGGGCTGGTAGC
CCAGGTGACGGACGAGGAAGCGTTAGCAGAGCGACTGGCGCAAGGCCCGA
TCGCGCTCTATTGCGGCTTCGATCCTACCGCTGACAGCTTGCATTTGGGG
CATCTTGTTCCATTGTTATGCCTGAAACGCTTCCAGCAGGCGGGCCACAA
GCCGGTTGCGCTGGTAGGCGGCGCGACGGGTCTGATTGGCGACCCGAGCT
TCAAAGCTGCCGAGCGTAAGCTGAACACCGAAGAAACTGTTCAGGAGTGG
GTGGACAAAATCCGTAAGCAGGTTGCCCCGTTCCTCGATTTCGACTGTGG
AGAAAACTCTGCTATCGCGGCGAACAACTATGACTGGTTCGGCAATATGA
ATGTGCTGACCTTCCTGCGCGATATTGGCAAACACTTCTCCGTTAACCAG
ATGATCAACAAAGAAGCGGTTAAGCAGCGTCTCAACCGTGAAGATCAGGG
GATTTCGTTCACTGAGTTTTCCTACAACCTGTTGCAGGGTTATGATTTTG
CCTGTTTGAACAAACAGTACGGTGTGGTGCTGCAAATTGGTGGTTCTGAC
CAGTGGGGTAACATCACTTCTGGTATCGACCTGACCCGTCGTCTGCATCA
GAATCAGGTGTTTGGCCTGACCGTTCCGCTGATCACTAAAGCAGATGGCA
CCAAATTTGGTAAAACTGAAGGCGGCGCAGTCTGGTTGGATCCGAAGAAA
ACCAGCCCGTACAAATTCTACCAGTTCTGGATCAACACTGCGGATGCCGA
CGTTTACCGCTTCCTGAAGTTCTTCACCTTTATGAGCATTGAAGAGATCA
ACGCCCTGGAAGAAGAAGATAAAAACAGCGGTAAAGCACCGCGCGCCCAG
TATGTACTGGCGGAGCAGGTGACTCGTCTGGTTCACGGTGAAGAAGGTTT
ACAGGCGGCAAAACGTATTACCGAATGCCTGTTCAGCGGTTCTTTGAGTG
CGCTGAGTGAAGCGGACTTCGAACAGCTGGCGCAGGACGGCGTACCGATG
GTTGAGATGGAAAAGGGCGCAGACCTGATGCAGGCACTGGTCGATTCTGA
ACTGCAACCTTCCCGTGGTCAGGCACGTAAAACTATCGCCTCCAATGCCA
TCACCATTAACGGTGAAAAACAGTCCGATCCTGAATACTTCTTTAAAGAA
GAAGATCGTCTGTTTGGTCGTTTTACCTTACTGCGTCGCGGTAAAAAGAA
TTACTGTCTGATTTGCTGGAAATAA.

By “wild type (WT) leucyl-tRNA synthetase (leuRS) polypeptide sequence” is meant a polypeptide or fragment thereof having at least about 85% amino acid identity to GenBank Accession No. QPN94495.1 and having activities that include charging a tRNA molecule with an amino acid. In some embodiments, the amino acid is leucine. A representative WT leuRS polypeptide sequence is provided below:

(SEQ ID NO: 4)
MQEQYRPEEIESKVQLHWDEKRTFEVTEDESKEKYYCLSMLPYPSGRLHM
GHVRNYTIGDVIARYQRMLGKNVLQPIGWDAFGLPAEGAAVKNNTAPAPW
TYDNIAYMKNQLKMLGFGYDWSRELATCTPEYYRWEQKFFTELYKKGLVY
KKTSAVNWCPNDQTVLANEQVIDGCCWRCDTKVERKEIPQWFIKITAYAD
ELLNDLDKLDHWPDTVKTMQRNWIGRSEGVEITFNVNDYDNTLTVYTTRP
DTFMGCTYLAVAAGHPLAQKAAENNPELAAFIDECRNTKVAEAEMATMEK
KGVDTGFKAVHPLTGEEIPVWAANFVLMEYGTGAVMAVPGHDQRDYEFAS
KYGLNIKPVILAADGSEPDLSQQALTEKGVLFNSGEFNGLDHEAAFNAIA
DKLTAMGVGERKVNYRLRDWGVSRQRYWGAPIPMVTLEDGTVMPTPDDQL
PVILPEDVVMDGITSPIKADPEWAKTTVNGMPALRETDTFDTFMESSWYY
ARYTCPQYKEGMLDSEAANYWLPVDIYIGGIEHAIMHLLYFRFFHKLMRD
AGMVNSDEPAKQLLCQGMVLADAFYYVGENGERNWVSPVDAIVERDEKGR
IVKAKDAAGHELVYTGMSKMSKSKNNGIDPQVMVERYGADTVRLFMMFAS
PADMTLEWQESGVEGANRFLKRVWKLVYEHTAKGDVAALNVDALTENQKA
LRRDVHKTIAKVTDDIGRRQTFNTAIAAIMELMNKLAKAPTDGEQDRALM
QEALLAVVRMLNPFTPHICFTLWQELKGEGDIDNAPWPVADEKAMVEDST
LVVVQVNGKVRAKITVPVDATEEQVRERAGQEHLVAKYLDGVTVRKVIYV
PGKLLNLVVG.

By “wild type (WT) leucyl-tRNA synthetase (leuRS) polynucleotide sequence” is meant a polynucleotide or fragment thereof having at least about 85% amino acid identity to a nucleotide sequence corresponding to base pairs 3344203 to 3346785 of GenBank Accession No. CP058342.1 and encoding a polypeptide having activities that include charging a tRNA molecule with an amino acid. In some embodiments, the amino acid is leucine. A representative WT leuRS polynucleotide sequence is provided below:

(SEQ ID NO: 5)
ATGCAAGAGCAATACCGCCCGGAAGAGATAGAATCCAAAGTACAGCTTCA
TTGGGATGAGAAGCGCACATTTGAAGTAACCGAAGACGAGAGCAAAGAGA
AGTATTACTGCCTGTCTATGCTTCCCTATCCTTCTGGTCGACTACACATG
GGCCACGTACGTAACTACACCATCGGTGACGTGATCGCCCGCTACCAGCG
TATGCTGGGCAAAAACGTCCTGCAGCCGATCGGCTGGGACGCGTTTGGTC
TGCCTGCGGAAGGCGCGGCGGTGAAAAACAACACCGCTCCGGCACCGTGG
ACGTACGACAACATCGCGTATATGAAAAACCAGCTCAAAATGCTGGGCTT
TGGTTATGACTGGAGCCGCGAGCTGGCAACCTGTACGCCGGAATACTACC
GTTGGGAACAGAAATTCTTCACCGAGCTGTATAAAAAAGGCCTGGTATAT
AAGAAGACTTCTGCGGTCAACTGGTGCCCGAACGACCAGACCGTACTGGC
GAACGAACAAGTTATCGACGGCTGCTGCTGGCGCTGCGATACCAAAGTTG
AACGTAAAGAGATCCCGCAGTGGTTTATCAAAATCACTGCTTACGCTGAC
GAGCTGCTCAACGATCTGGATAAACTGGATCACTGGCCAGACACCGTTAA
AACCATGCAGCGTAACTGGATCGGTCGTTCCGAAGGCGTGGAGATCACCT
TCAACGTTAACGACTATGACAACACGCTGACCGTTTACACTACCCGCCCG
GACACCTTTATGGGTTGTACCTACCTGGCGGTAGCTGCGGGTCATCCGCT
GGCGCAGAAAGCGGCGGAAAATAATCCTGAACTGGCGGCCTTTATTGACG
AATGCCGTAACACCAAAGTTGCCGAAGCTGAAATGGCGACGATGGAGAAA
AAAGGCGTCGATACTGGCTTTAAAGCGGTTCACCCATTAACGGGCGAAGA
AATTCCCGTTTGGGCAGCAAACTTCGTATTGATGGAGTACGGCACGGGCG
CAGTTATGGCGGTACCGGGGCACGACCAGCGCGACTACGAGTTTGCCTCT
AAATACGGCCTGAACATCAAACCGGTTATCCTGGCAGCTGACGGCTCTGA
GCCAGATCTTTCTCAGCAAGCCCTGACTGAAAAAGGCGTGCTGTTCAACT
CTGGCGAGTTCAACGGTCTTGACCATGAAGCGGCCTTCAACGCCATCGCC
GATAAACTGACTGCGATGGGCGTTGGCGAGCGTAAAGTGAACTACCGCCT
GCGCGACTGGGGTGTTTCCCGTCAGCGTTACTGGGGCGCGCCGATTCCGA
TGGTGACGCTGGAAGACGGTACCGTAATGCCGACCCCGGACGACCAGCTG
CCGGTGATCCTGCCGGAAGATGTGGTAATGGACGGCATTACCAGCCCGAT
TAAAGCAGATCCGGAGTGGGCGAAAACTACCGTTAACGGTATGCCAGCAC
TGCGTGAAACCGACACTTTCGACACCTTTATGGAGTCCTCCTGGTACTAT
GCGCGCTACACTTGCCCGCAGTACAAAGAAGGTATGCTGGATTCCGAAGC
GGCTAACTACTGGCTGCCGGTGGATATCTACATTGGTGGTATTGAACACG
CCATTATGCACCTGCTCTACTTCCGCTTCTTCCACAAACTGATGCGTGAT
GCAGGCATGGTGAACTCTGACGAACCAGCGAAACAGTTGCTGTGTCAGGG
TATGGTGCTGGCAGATGCCTTCTACTATGTTGGCGAAAACGGCGAACGTA
ACTGGGTTTCCCCGGTTGATGCTATCGTTGAACGTGACGAGAAAGGCCGT
ATCGTGAAAGCGAAAGATGCGGCAGGCCATGAACTGGTTTATACCGGCAT
GAGCAAAATGTCCAAGTCGAAGAACAACGGTATCGACCCGCAGGTGATGG
TTGAACGTTACGGCGCGGACACCGTTCGTCTGTTTATGATGTTTGCTTCT
CCGGCTGATATGACTCTCGAATGGCAGGAATCCGGTGTGGAAGGGGCTAA
CCGCTTCCTGAAACGTGTCTGGAAACTGGTTTACGAGCACACAGCAAAAG
GTGATGTTGCGGCACTGAACGTTGATGCGCTGACTGAAAATCAGAAAGCG
CTGCGTCGCGATGTGCATAAAACGATCGCTAAAGTGACCGATGATATCGG
CCGTCGTCAGACCTTCAACACCGCAATTGCGGCGATTATGGAGCTGATGA
ACAAACTGGCGAAAGCACCAACCGATGGCGAGCAGGATCGCGCTCTGATG
CAGGAAGCACTGCTGGCCGTTGTCCGTATGCTTAACCCGTTCACCCCGCA
CATCTGCTTCACGCTGTGGCAGGAACTGAAAGGCGAAGGCGATATCGACA
ACGCGCCGTGGCCGGTTGCTGACGAAAAAGCGATGGTGGAAGACTCCACG
CTGGTCGTGGTGCAGGTTAACGGTAAAGTCCGTGCCAAAATCACCGTTCC
GGTGGACGCAACGGAAGAACAGGTTCGCGAACGTGCTGGCCAGGAACATC
TGGTAGCAAAATATCTTGATGGCGTTACTGTACGTAAAGTGATTTACGTA
CCAGGTAAACTCCTCAATCTGGTCGTTGGCTAA.

By “4-benzoyl-1-phenylalanine (Bpa)” is meant a noncanonical amino acid with the structure

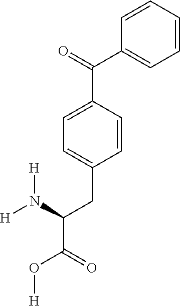 [SMILES: N[C@@H](Cc1ccc(C(=O)c2ccccc2)cc1)C(=O)O]

or an analog thereof.

By “O-methyl-L-tyrosine (OmeY)” is meant a noncanonical amino acid with the structure

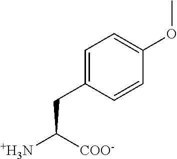

or an analog thereof.

By “p-acetyl-L-phenylalanine (AcF)” is meant a noncanonical amino acid with the structure

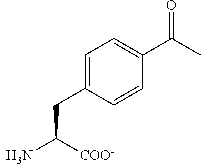

or an analog thereof.

By “p-azido-L-phenylalanine (AzF)” is meant a noncanonical amino acid with the structure

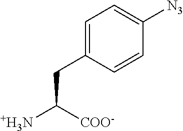

or an analog thereof.

By “p-propargyloxy-L-phenylalanine (OPG)” is meant a noncanonical amino acid with the structure

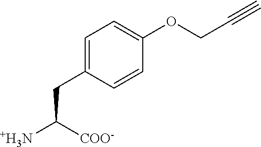

or an analog thereof.

By “4-azidomethyl-L-phenylalanine (AzMF)” is meant a noncanonical amino acid with the structure

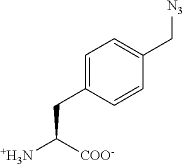

or an analog thereof.

By “4-borono-L-phenylalanine (BPhe)” is meant a noncanonical amino acid with the structure

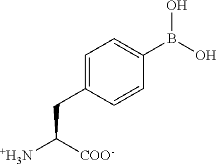

or an analog thereof.

By “3,4-dihydroxy-L-phenylalanine (DOPA)” is meant a noncanonical amino acid with the structure

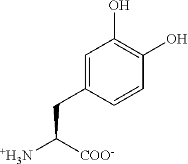

or an analog thereof.

By “4-iodo-L-phenylalanine (IPhe)” is meant a noncanonical amino acid with the structure

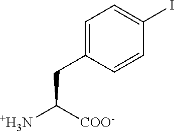

or an analog thereof.

By “L-α-aminocaprylic acid (AC)” is meant a noncanonical amino acid with the structure

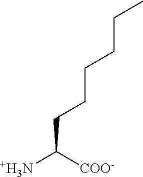

or an analog thereof.

By “NE-azido-L-lysine (AzK)” is meant a noncanonical amino acid with the structure

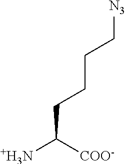

or an analog thereof.

By “3-Amino-L-tyrosine (ATyr)” is meant a noncanonical amino acid with the structure

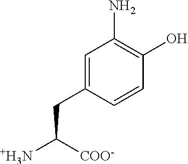

or an analog thereof.

By “4-Amino-L-phenylalanine (APhe)” is meant a noncanonical amino acid with the structure

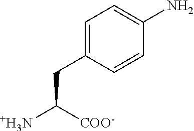

or an analog thereof.

By “dimethyl-L-lysine (DMK)” is meant a noncanonical amino acid with the structure

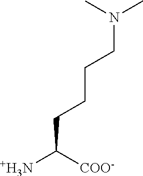

or an analog thereof.

By “Boc-L-lysine (BocK)” is meant a noncanonical amino acid with the structure

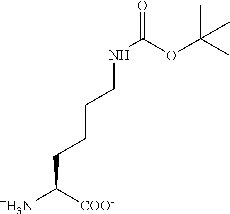

or an analog thereof.

By “(S)-2-amino-6-((2-azidoethoxy)carbonylamino)hexanoic acid (LysN3)” is meant a noncanonical amino acid with the structure

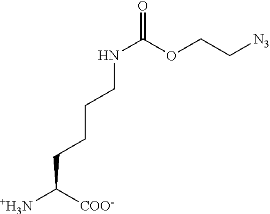

or an analog thereof.

By “2-Amino-6-(prop-2-ynoxycarbonylamino)hexanoic acid (LysAlk)” is meant a noncanonical amino acid with the structure

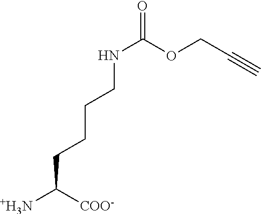

or an analog thereof. By “O-Sulfo-L-tyrosine (SY)” is meant a noncanonical amino acid with the structure

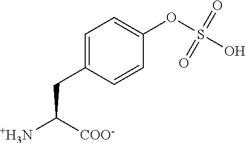

or an analog thereof.

By “2-amino-3-[4-(carboxymethyl) phenyl]propanoic acid (CMF)” is meant a noncanonical amino acid with the structure

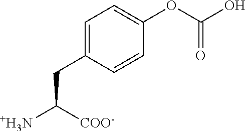

or an analog thereof.

By “L-p-hydroxy-phenyllactic acid (Ester)” is meant a noncanonical amino acid with the structure

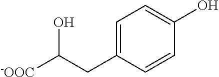

or an analog thereof.

By “L-2-Amino-4-phosphonobutyric acid (PSA)” is meant a noncanonical amino acid with the structure

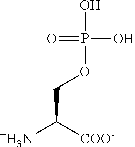

or an analog thereof.

By “O-phospho-L-serine (OPS)” is meant a noncanonical amino acid with the structure

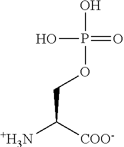

or an analog thereof.

By “Acetyl-L-lysine (AcK)” is meant a noncanonical amino acid with the structure

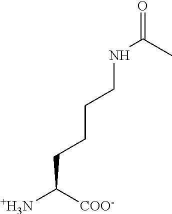

or an analog thereof.

By “N⁶-((2-(3-methyl-3H-diazirin-3-yl)ethoxy)carbonyl)-L-lysine (Photo-Lysine, Phk)” or “H-L-photo-lysine” is meant a noncanonical amino acid with the structure

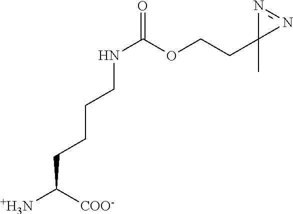

or an analog thereof.

By “O-(2-Bromoethyl)-L-tyrosine (Obey)” is meant a noncanonical amino acid with the structure

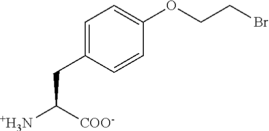

or an analog thereof.

By “agent” is meant any small molecule chemical compound, antibody, nucleic acid molecule, or polypeptide, or fragments thereof.

By “alteration” is meant a change (increase or decrease) in the expression level, structure, or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. In some embodiments, the alteration in structure is one or more amino acid changes.

By “aminoacyl-tRNA synthetase” is meant a polypeptide or fragment thereof that catalyzes the aminoacylation of transfer RNAs. In some embodiments, this activity is referred to as “charging a tRNA molecule with an amino acid.” In various embodiments, the amino acid is a noncanonical amino acid.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid. An analog may include a solvate or salt of a compound. An analog may include a freebase or free acid form of a compound.

By “to charge” or “charging” or “tRNA charging” with respect to an amino acid is meant aminoacylation of a tRNA molecule using the amino acid. In various embodiments, the amino acid is a noncanonical amino acid (ncAA).

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3500, 4000, 4500, 5000, 5500, 6000, or 6500 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increases” is meant a positive alteration of at least 10%, 25%, 50%, 75%, or 100%.

By “in vitro translation system” or “cell-free translation system” is meant a system for production of a protein using biological machinery in a cell-free system. In embodiments, an in vitro translation system contains a cell extract, an energy source, a supply of amino acids, cofactors (e.g., magnesium), and a polynucleotide sequence (e.g., an mRNA transcript or a polynucleotide sequence encoding a polypeptide). In various embodiments, an in vitro translation system contains the components necessary for the production of a protein from an mRNA transcript (e.g., ribosomes, aminoacyl-tRNA synthetases, elongation factors, nucleases, etc.). Non-limiting examples of in vitro translation systems include those derived from E. coli, rabbit reticulocytes, wheat germ, insect cells, and yeast (e.g., Kluyveromyces (the D2P system), all of which are commercially available. In vitro translation systems include those described in Khambhati, K., et al., “Exploring the potential of cell-free protein synthesis for extending the abilities of biological systems,” Bioeng. Biotechnol. vol. 7, art. 248 (2019), doi: 10.3389/fbioe.2019.00248, the disclosure of which is incorporated herein by reference in its entirety for all purposes.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, mass spectroscopy, polyacrylamide gel electrophoresis, or by HPLC analysis.

By “noncanonical amino acid” is meant an amino acid analog that acts as a surrogate for a naturally occurring amino acid. In one embodiment, a noncanonical amino acid is an isostructural analog of a canonical amino acid. The terms “noncanonical amino acid”, “unnatural amino acid”, “nonnatural amino acid”, “nonstandard amino acid”, or “nonproteinogenic amino acid” are interchangeable. In embodiments, a noncanonical amino acid is an amino acid not naturally encoded in the genome of an organism. Non-limiting examples of noncanonical amino acids include O-methyl-L-tyrosine (OmeY); p-acetyl-L-phenylalanine (AcF); p-azido-L-phenylalanine (AzF); p-propargyloxy-L-phenylalanine (OPG); 4-azidomethyl-L-phenylalanine (AzMF); 4-borono-L-phenylalanine (BPhe); 3,4-dihydroxy-L-phenylalanine (DOPA); O-(2-Bromoethyl)-tyrosine (Obey); 4-iodo-L-phenylalanine (IPhe); L-α-aminocaprylic acid (AC); Nc-azido-L-lysine (AzK); 3-Amino-L-tyrosine (ATyr); 4-Amino-L-phenylalanine (APhe); dimethyl-L-lysine (DMK); Boc-L-lysine (BocK); (S)-2-amino-6-((2-azidoethoxy)carbonylamino)hexanoic acid (LysN3); and 2-Amino-6-(prop-2-ynoxycarbonylamino)hexanoic acid (LysAlk), O-(2-Bromoethyl)-L-tyrosine, O-Sulfo-L-tyrosine (SY), 2-amino-3-[4-(carboxymethyl) phenyl]propanoic acid (CMF), L-p-hydroxy-phenyllactic acid (Ester), L-2-Amino-4-phosphonobutyric acid (PSA), O-phospho-L-serine (OPS), Acetyl-L-lysine (AcK), 4-benzoyl-1-phenylalanine (Bpa), and N⁶-((2-(3-methyl-3H-diazirin-3-yl)ethoxy)carbonyl)-L-lysine (Photo-Lysine, Phk).

As used herein, “obtaining” as in “obtaining an agent” includes synthesizing, purchasing, or otherwise acquiring the agent.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some aspects the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIGS. 1A-ID are a schematic, protein structural images, and flow cytometry dot plots relating to E. coli tyrosyl-tRNA synthetase (TyrRS) and leucyl-tRNA synthetase (LeuRS) library construction and fluorescence-activated cell sorting (FACS). FIG. 1A provides a schematic representation of the yeast display noncanonical amino acid (ncAA) incorporation reporter plasmid (Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269). and the E. coli Tyrosyl-tRNA synthetase (TyrRS) and Leucyl tRNA synthetase (LeuRS) genes and the cognate tRNA for amber (TAG) codon suppression.

FIG. 1B provides an image of the E. coli Tyrosyl-tRNA synthetase (TyrRS) crystal structure (PDB 6HB5) with highlighted residues in the active site chosen for mutation. Residues and degenerate codons with library mutations are listed in the table on the right. Wildtype (WT) Tyrosyl-tRNA synthetase (TyrRS) residues were included, in addition to degenerate codons, at positions 37 and 183. Degenerate codon VNK encodes the following residues: Leu, Pro, His, Gln, Arg, Ile, Met, Thr, Asn, Lys, Ser, Val, Ala, Asp, Glu, and Gly. Degenerate codon RRT encodes the following residues: Asn, Ser, Asp, and Gly. Degenerate codon KYA encodes the following residues: Leu, Ser, Val, and Ala. FIG. 1C provides an image of the E. coli Leucyl tRNA synthetase (LeuRS) crystal structure (PDB 4CQN) with highlighted residues in the active site chosen for mutation. An editing domain T252A mutation was included in the library to reduce misincorporation of Leu. Residues and degenerate codons with library mutations are listed in the table on the right. Degenerate codon RST encodes the following residues: Thr, Ala, Ser, and Gly. Degenerate codons NNY and NNT encode the following residues: Phe, Ser, Tyr, Cys, Leu, Pro, His, Arg, Ile, Thr, Asn, Val, Ala, Asp, and Gly. FIG. 1D provides flow cytometry dot plots corresponding to example fluorescence-activated cell sorting (FACS) screens of the pooled Tyrosyl-tRNA synthetase (TyrRS) and Leucyl tRNA synthetase (LeuRS) libraries for noncanonical amino acid (ncAA) LysN3. Screens with flow cytometry-based checks between rounds allowed for progression of screening with either a positive or negative round as needed until a population with low cAA and high noncanonical amino acid (ncAA) incorporation was attained. DNA from the population was purified and sequenced, and individual aminoacyl-tRNA synthetase (aaRS) mutants were further evaluated for efficiency and fidelity. Throughout the figures, ncAA represents “noncanonical amino acid”, and aaRS represents “aminoacyl-tRNA synthetase”, TyrRS represents “tyrosyl-tRNA synthetase”, LeuRS represents “leucyl-tRNA synthetase”, and cAA represents “canonical amino acid”.

FIG. 2 provides structures for a set of noncanonical amino acids used in the Examples. 1: O-methyl-L-tyrosine (OmeY); 2: p-acetyl-L-phenylalanine (AcF); 3: p-azido-L-phenylalanine (AzF); 4: p-propargyloxy-L-phenylalanine (OPG); 5: 4-azidomethyl-L-phenylalanine (AzMF); 6: 4-borono-L-phenylalanine (BPhe); 7: 3,4-dihydroxy-L-phenylalanine (DOPA); 8: 4-iodo-L-phenylalanine (IPhe); 9: L-α-aminocaprylic acid (AC); 10: Nε-azido-L-lysine (AzK); 11: 3-Amino-L-tyrosine (ATyr); 12: 4-Amino-L-phenylalanine (APhe); 13: dimethyl-L-lysine (DMK); 14: Boc-L-lysine (BocK); 15: (S)-2-amino-6-((2-azidoethoxy)carbonylamino)hexanoic acid (LysN3); 16: 2-Amino-6-(prop-2-ynoxycarbonylamino)hexanoic acid (LysAlk).

FIGS. 3A and 3B present bar plots and flow cytometry dot plots relating to aaRSs (aminoacyl-tRNA synthetases) from fluorescence-activated cell sorting (FACS) that can encode unique ncAAs (noncanonical amino acids). FIG. 3A presents bar graphs showing relative readthrough efficiency and maximum misincorporation frequency for aaRSs (aminoacyl-tRNA synthetases) isolated from fluorescence-activated cell sorting (FACS) that are able to encode unique ncAAs (noncanonical amino acids) OmeY, BPhe, DOPA, ATyr, LysN3, and OPG. FIG. 3B presents flow cytometry dot plots for each of the aminoacyl-tRNA synthetases (aaRSs) in panel FIG. 3A. Only one of three biological replicates is shown for each condition (-ncAA and +ncAA). Full-length detection is shown on the X axis and display detection is shown on the Y axis. An example of wildtype (WT) display is shown for comparison.

FIGS. 4A-4C provide bar graphs and a structural image relating to performance of error-prone polymerase chain reaction (epPCR) aminoacyl-tRNA synthetase (aaRS) mutants. FIG. 4A provides a bar graph showing relative readthrough efficiency of three error-prone polymerase chain reaction (epPCR) mutants that outperformed the parent DOPARS efficiency of DOPA incorporation at 0.1 mM, 1 mM, or both 0.1 and 1 mM DOPA. FIG. 4B provides a bar graph showing maximum misincorporation frequency of three error-prone polymerase chain reaction (epPCR) mutants that were comparable to the parent DOPARS fidelity of DOPA incorporation at both 0.1 and 1 mM DOPA. FIG. 4C is an image of the crystal structure of an E. coli Tyrosyl-tRNA synthetase (TyrRS) with mutated residues from error-prone polymerase chain reaction (epPCR) highlighted in blue. The crystal structure in this figure was derived from PDB 6HB5.

FIGS. 5A and 5B present bar plots showing relative readthrough efficiency (RRE) and maximum misincorporation frequency (MMF) of polyspecific and specific aaRSs (aminoacyl-tRNA synthetases) from fluorescence-activated cell sorting (FACS). FIG. 5A presents bar plots relating to the evaluation of relative readthrough efficiency (RRE) and maximum misincorporation frequency (MMF) of three specific aminoacyl-tRNA synthetases (aaRSs) (S-OPGRS-3, S-OPGRS-7, and S-OPGRS-9) and four polyspecific aminoacyl-tRNA synthetases (aaRSs) (PolyT1RS-7, PolyT2RS-5, PolyT2RS-6, and PolyT2RS-7) with a series of six aromatic ncAAs (noncanonical amino acids) used for screening via fluorescence-activated cell sorting (FACS). FIG. 5B presents bar plots relating to the evaluation of relative readthrough efficiency (RRE) and maximum misincorporation frequency (MMF) of six aminoacyl-tRNA synthetases (aaRSs) originating from a Track 1 polyspecificity screen that encode aliphatic noncanonical amino acids (ncAAs) LysAlk, LysN3, and BocK. All relative readthrough efficiency (RRE) and maximum misincorporation frequency (MMF) values were derived from samples in biological triplicate and error bars represent the standard deviation of triplicate values that was propagated during relative readthrough efficiency (RRE) and maximum misincorporation frequency (MMF) calculations.

FIGS. 6A and 6B are heatmaps presenting a comprehensive evaluation of aminoacyl-tRNA synthetase (aaRS) activity with aromatic and aliphatic noncanonical amino acids (ncAAs). FIG. 6A provides heat maps showing relative readthrough efficiency and maximum misincorporation efficiency for eight aminoacyl-tRNA synthetases (aaRSs) with 10 aromatic noncanonical amino acids (ncAAs). FIG. 6B provides heat maps showing relative readthrough efficiency and maximum misincorporation efficiency for six aminoacyl-tRNA synthetases (aaRSs) with six aliphatic noncanonical amino acids (ncAAs). In general, a high relative readthrough efficiency (RRE) value corresponds to more efficient noncanonical amino acid (ncAA) incorporation with 0=almost all reporter proteins truncated at TAG codon and 1=WT (wild type) protein translation efficiency. Maximum misincorporation frequency (MMF) is a measure of the canonical amino acid (cAA) misincorporation of the aminoacyl-tRNA synthetase (aaRS) at the TAG codon but does not directly indicate a percent of reporter proteins that contain a canonical amino acid (cAA) at the TAG codon. Rather, maximum misincorporation frequency (MMF) provides an approximation of the highest possible canonical amino acid (cAA) misincorporation in the worst-case scenario; e.g., when there is no canonical amino acid (cAA) present. All relative readthrough efficiency (RRE) and maximum misincorporation frequency (MMF) values were derived from samples in biological triplicate and error bars represent the standard deviation of triplicate values that was propagated during relative readthrough efficiency (RRE) and maximum misincorporation frequency (MMF) calculations.

FIG. 7 is a schematic providing a map of E. coli aminoacyl-tRNA synthetase (aaRS) pooled library sorting.

FIGS. 8A and 8B presents flow cytometry dot plots and noncanonical amino acid structures corresponding to Track 1 aaRS polyspecificity sorts induced in the presence of no ncAAs and 21 distinct ncAAs at 1 mM final concentration. FIG. 8A presents the flow cytometry dot plots and FIG. 8B presents the corresponding noncanonical amino acid structures and names.

FIG. 9 presents flow cytometry dot plots for PolyT2RS-5 demonstrating qualitative readthrough of the reporter protein in the presence of 1 mM APhe compared to no readthrough in the presence of no ncAAs.

FIG. 10 is a comparison of the relative readthrough efficiency (RRE) and maximum misincorporation frequency (MMF) of an acetylphenylalanyl-tRNA synthetase (AcFRS) with and without an I7M (isoleucine to methionine at position 7 in the protein) mutation, induced in the presence of 1 mM AzF.

FIGS. 11A-11T provide plots showing MALDI mass spectrometry characterization of ncAA-containing tryptic digested peptide fragments. FIG. 11A, Wildtype (WT) Donkey1.1 reporter protein used to evaluate ncAA incorporation via MALDI MS. FIG. 11B, Donkey1.1-H54TAG reporter protein with LeuOmeRS induced with 1 mM OmeY. FIG. 11C, Donkey1.1-H54TAG with A-OmeRS-7 induced with 1 mM OmeY. FIG. 11D, Donkey1.1-H54TAG with A-DOPARS-4 induced with 1 mM DOPA. FIG. 11E, Donkey1.1-H54TAG with DOPARS-0.1-10 induced with 1 mM DOPA. FIG. 11F, Donkey1.1-H54TAG with A-BPheRS-2 induced with 1 mM BPhe. FIG. 11G, Repeated MALDI MS for Donkey1.1-H54TAG with A-BPheRS-2 induced with 1 mM BPhe. FIG. 11H, Donkey1.1-H54TAG with A-ATyrRS-1 induced with 1 mM ATyr. FIG. 11I, Donkey1.1-H54TAG with A-LysN3RS-1 induced with 1 mM LysN3. FIG. 11J, Donkey1.1-H54TAG with B-LysN3RS-7 induced with 1 mM LysN3. FIG. 11K, Donkey1.1-H54TAG with B-LysAlkRS-3 induced with 1 mM LysAlk. FIG. 11L, Donkey1.1-H54TAG with B-BocKRS-2 induced with 1 mM BocK. FIG. 11M, Donkey1.1-H54TAG with B-OPGRS-L6 induced with 1 mM OPG. FIG. 11N, Donkey1.1-H54TAG with SpecOPGRS-3 induced with 1 mM OPG. FIG. 11O, Donkey1.1-H54TAG with PolyT2RS-5 induced with 1 mM AcF. FIG. 11P, Donkey1.1-H54TAG with PolyT2RS-5 induced with 1 mM AzF. FIG. 11Q, Donkey1.1-H54TAG with PolyT2RS-5 induced with 1 mM OmeY. FIG. 11R, Donkey1.1-H54TAG with PolyT2RS-5 induced with 1 mM OPG. FIG. 11S, Donkey1.1-H54TAG with PolyT2RS-5 induced with 1 mM AzMF. FIG. 11T, Donkey1.1-H54TAG with PolyT2RS-5 induced with 1 mM IPhe.

FIGS. 12A-12C provide schematics and a bar graph. FIG. 12A provides a schematic showing the structures of noncanonical amino acids (ncAA). The reactive groups are circled.

FIG. 12B provides a schematic showing constructs used for expression of fluorescent reporter, BXG, and the amber suppression machinery. The BXG reporter consisted of an N-terminal fluorescent protein (BFP) and a C-terminal green fluorescent protein (GFP) tethered by a linker containing an amber codon TAG. FIG. 12C provides a bar graph showing GFP detection in yeast cells induced in the absence (-ncAA) or presence of 1 mM of each indicated ncAA. Bars indicate the median fluorescence intensity (MFI) of GFP in populations expressing BFP as determined by flow cytometry analysis. Error bars correspond to the robust coefficient of variation of such populations. This screening experiment was performed once. In FIG. 12C each group of 4 bars corresponds to “-ncAA”, “Bpa”, “Phk”, and “Obey” from left-to-right, respectively.

FIGS. 13A-13C provide flow cytometry dot plots, formulae, and bar graphs. FIG. 13A provides a representative example of dot plots obtained through flow cytometry analysis of the BYG or BXG yeast populations induced in the absence (-ncAA) or presence of 1 mM ncAA (here shown for the Obey and OPGRS-L6 system). FIG. 13B provides formulae used to calculate the relative readthrough efficiency (RRE) and maximum misincorporation frequency (MMF) based on the fluorescent reporters. FIG. 13C provides bar graphs showing RRE and MMF values of the selected synthetases (Obey and Phk) and a known active synthetase (BpaRS).

### DETAILED DESCRIPTION OF THE INVENTION

The invention features compositions and methods that are useful for incorporation of noncanonical amino acids into polypeptide sequences. In particular, the invention of the disclosure provides aminoacyl-tRNA synthetases, compositions thereof, and methods for use thereof.

The invention of the present disclosure is based at least in part upon the development of a high-throughput screening platform in S. cerevisiae to evolve aminoacyl-tRNA synthetases (aaRSs) that were able to charge noncanonical amino acids (ncAAs) that had not previously been encoded in proteins (e.g., in yeast).

### Synthetic Biology

The ability to produce chemically versatile proteins with encoded noncanonical amino acids (ncAAs) at specific sites supports a growing number of applications in chemical and synthetic biology. Unique noncanonical amino acid (ncAA) functional groups allow for exploration of post-translational modifications, examination of protein-protein interactions, and discovery of biological therapeutics and protein medicinal chemistry, among many other applications. Aminoacyl-tRNA synthetases (aaRSs) evolved to maintain the fidelity of genetic code translation—that is, to precisely charge canonical amino acids (cAAs) to their cognate tRNAs while discriminating against other potential substrates. However, it is possible to engineer and express these precise aminoacyl-tRNA synthetases (aaRSs) in organisms from different domains of life to charge noncanonical amino acids (ncAAs) without interacting with the host cell's natural translation machinery. These orthogonal aaRS/tRNA pairs can be engineered to improve the efficiency of noncanonical amino acid (ncAA) incorporation in proteins in both prokaryotic and eukaryotic hosts. Diversification is generally limited to residues in the active site with selections most commonly used to isolate aminoacyl-tRNA synthetases (aaRSs) with desirable properties. Selection criteria are generally limited to incorporation of a target noncanonical amino acid (ncAA) and absence of incorporation of cAAs, without attempts to isolate aminoacyl-tRNA synthetases (aaRSs) with particular specificity or polyspecificity profiles. For example, when expressing proteins with dual- or triple-noncanonical amino acid (ncAA) incorporation, additional selection for aminoacyl-tRNA synthetase (aaRS) mutants that do not non-specifically charge non-target noncanonical amino acids (ncAAs) is sometimes necessary. The extent of specificity or polyspecificity of aminoacyl-tRNA synthetases (aaRSs) that can be engineered remains unknown, particularly as it is not well understood how mutations in or outside of the active sites of aminoacyl-tRNA synthetases (aaRSs) contribute to these two properties.

One property of aminoacyl-tRNA synthetases (aaRSs) beyond their ability to maintain the fidelity of the genetic code is their potential to demonstrate specificity or polyspecificity when engineered to charge noncanonical amino acids (ncAAs) to their cognate tRNAs. Many methods for evolving orthogonal translation machinery utilize life-or-death assays. Selection strategies in Escherichia coli involve the use of a toxic Barnase gene containing the codon at which a noncanonical amino acid (ncAA) could be encoded (the undesired readthrough of which leads to cell death, for negative selection) and a beta-lactamase or chloramphenicol acetyltransferase gene with the same codon (the desired readthrough of which would lead to cell survival in the presence of ampicillin, for positive selection). In yeast, a similar but unique selection strategy is used, where the yeast selection strain Saccharomyces cerevisiae MaV203 and the transcriptional activator GAL4 gene containing two TAG codons are used to measure responses in three reporter genes (HIS3, URA3, and lacZ) when both TAG codons have been successfully suppressed. These selection strategies offer several advantages, but are more difficult to employ in higher eukaryotes, such as mammalian cells. However, some archaeal aminoacyl-tRNA synthetases (aaRSs) such as the M. mazei or M. barkeri pyrrolysyl-tRNA synthetases (PylRSs) have been reported to be orthogonal in E. coli, S. cerevisiae, and mammalian cells. These selection and screening methods have changed the landscape of noncanonical amino acid (ncAA) incorporation as a field. During the course of attempts to improve aminoacyl-tRNA synthetase (aaRS) activity for hundreds of noncanonical amino acids (ncAAs), researchers have discovered and begun to probe characteristics beyond simply the ability to encode a novel noncanonical amino acid (ncAA). Despite the success of engineering various aminoacyl-tRNA synthetases (aaRSs) for improved noncanonical amino acid (ncAA) recognition and activity in E. coli, relatively little work has been done to develop E. coli or archaeal aminoacyl-tRNA synthetases (aaRSs) in yeast. Evolving aminoacyl-tRNA synthetases (aaRSs) for improved activity or selectivity in yeast has been restricted to selections and low-throughput screens, despite highly advantageous tools such as yeast display that make possible high throughput aminoacyl-tRNA synthetase (aaRS) engineering. Aminoacyl-tRNA synthetase (aaRS) characteristics such as solubility and specificity are known to play key roles for individual applications, but it is not yet clear how best to evolve aminoacyl-tRNA synthetases (aaRSs) to address those needs.

### Library Screening

Libraries of aminoacyl-tRNA synthetases (aaRSs) were constructed in yeast and screened for incorporation of unique noncanonical amino acids (ncAAs). From these screens, several aminoacyl-tRNA synthetase variants were isolated that support translation with noncanonical amino acids (ncAAs) not previously encoded in proteins in yeast. As described in the Examples provided herein below, an aminoacyl-tRNA synthetase with only moderate activity toward its cognate noncanonical amino acid (ncAA) was randomly mutated using error-prone PCR (epPCR) to provide for the selection of aminoacyl-tRNA synthetases having improved efficiency of noncanonical amino acid (ncAA) charging. Using a combination of epPCR with stringent screening conditions supported by the yeast display reporter platform, aminoacyl-tRNA synthetase variants were identified with double the efficiency of stop codon readthrough compared to the parent polypeptide.

To date, very little work in engineering aminoacyl-tRNA synthetases (aaRSs) has included evolution for incorporation of multiple distinct noncanonical amino acids (ncAAs). Further, there are many advantages of controlling the specificity of aminoacyl-tRNA synthetases (aaRSs). By including structurally similar noncanonical amino acids (ncAAs) during negative screening rounds, aminoacyl-tRNA synthetases (aaRSs) were identified that supported incorporation of a single noncanonical amino acid (ncAA) out of a group of six structurally similar aromatic noncanonical amino acids (ncAAs). At the same time, screening criteria was also altered to identify aminoacyl-tRNA synthetases (aaRSs) with polyspecific behavior for multiple similar noncanonical amino acids (ncAAs) and isolated aminoacyl-tRNA synthetases (aaRSs) that encoded all 6 noncanonical amino acid (ncAA) analogs at varying levels.

Polyspecific aminoacyl-tRNA synthetases (aaRSs) from these screens were also able to charge several aliphatic noncanonical amino acids (ncAAs), suggesting that the mutations in these aminoacyl-tRNA synthetases (aaRSs) imbued polyspecific characteristics beyond specificity to a select group of similar noncanonical amino acids (ncAAs). The unique ability to express several distinct proteins using a single polyspecific aminoacyl-tRNA synthetase or to produce a single protein at high efficiency using an aminoacyl-tRNA synthetase with engineered specificity supports protein engineering efforts in critical fields such as protein medicinal chemistry for discovery of biological therapeutics. Additionally, aminoacyl-tRNA synthetases (aaRSs) engineered with these unique polyspecific characteristics could expand the chemical versatility of libraries of proteins containing noncanonical amino acids (ncAAs), adding a potentially highly valuable technology to the protein engineering toolkit.

Expression of proteins with genetically encoded amino acids beyond the canonical 20 benefits a broad range of applications, from the development of biological therapeutics to fundamental biological studies to better understand how eukaryotic cells function. A major factor limiting the use of these noncanonical amino acids (ncAAs) is the lack of engineered cellular machinery, also known as orthogonal translation systems (OTSs), that supports efficient genetic code expansion at repurposed stop codons. The Examples provided herein demonstrate the use of a yeast display-based noncanonical amino acid (ncAA) incorporation reporter platform to screen libraries of Escherichia coli tyrosyl- and leucyl-tRNA synthetases in high throughput for 1) incorporation of new noncanonical amino acids (ncAAs), 2) a specificity profile in which an aminoacyl-tRNA synthetase encode only one out of a group of six noncanonical amino acid (ncAA) analogs, and 3) a polyspecific aminoacyl-tRNA synthetase capable of encoding all six noncanonical amino acid (ncAA) analogs. Using flow cytometry-based screens aminoacyl-tRNA synthetases (aaRSs) were isolated that can encode two noncanonical amino acids (ncAAs) that had not previously been genetically encoded in proteins in yeast: 3,4-dihydroxy-L-phenylalanine (DOPA) and 4-borono-L-phenylalanine (BPhe). To enhance specificity, libraries of orthogonal translation systems (OTSs) were induced in the presence of several “off-target” noncanonical amino acids (ncAAs) during negative screens to enrich for clones capable of discriminating between similar noncanonical amino acids (ncAAs).

The results presented in the Examples indicate the feasibility of identifying orthogonal translation systems (OTSs) that support expansion of the genetic code to include structurally related noncanonical amino acids (ncAAs) without causing loss of fidelity of the expanded code. To enhance polyspecificity, two strategies were pursued: (1) induction with several noncanonical amino acids (ncAAs) simultaneously, followed by positive screens; and (2) induction with individual different noncanonical amino acids (ncAAs) in successive rounds of positive screening. While each of these approaches yielded orthogonal translation systems (OTSs) exhibiting high polyspecificity while still limiting canonical amino acid misincorporation, greater control over the specific set of noncanonical amino acids (ncAAs) incorporated by variants was achieved using methodology (2). Unexpectedly, populations enriched via strategy (1) with aromatic noncanonical amino acids (ncAAs) included variants that supported high levels of translation for several aliphatic derivatives of lysine, suggesting that even relatively conservative attempts to enhance polyspecificity may result in clones with broad noncanonical amino acid (ncAA) incorporation capabilities. The use of quantitative yeast display reporters to engineer orthogonal translation systems (OTSs) has allowed for properties to be selected that would be difficult to identify using other methodologies. These results have important implications related to the fundamental properties and evolvability of orthogonal translation systems (OTSs), while access to orthogonal translation systems (OTSs) with diverse activities and specific or polyspecific properties may prove invaluable for a range of applications within chemical and synthetic biology.

### Aminoacyl-tRNA Synthetases

The present invention provides new aminoacyl-tRNA synthetases with improved characteristics. The aminoacyl-tRNA synthetases can be used for the specific incorporation of one or more noncanonical amino acids at a predetermined location(s) in a polypeptide. The noncanonical amino acid useful in the invention include, but are not limited to, the following: O-methyl-L-tyrosine (OmeY); p-acetyl-L-phenylalanine (AcF); p-azido-L-phenylalanine (AzF); p-propargyloxy-L-phenylalanine (OPG); 4-azidomethyl-L-phenylalanine (AzMF); 4-borono-L-phenylalanine (BPhe); 3,4-dihydroxy-L-phenylalanine (DOPA); O-(2-Bromoethyl)-tyrosine (Obey); 4-iodo-L-phenylalanine (IPhe); L-α-aminocaprylic acid (AC); NE-azido-L-lysine (AzK); 3-Amino-L-tyrosine (ATyr); 4-Amino-L-phenylalanine (APhe); dimethyl-L-lysine (DMK); Boc-L-lysine (BocK); (S)-2-amino-6-((2-azidoethoxy)carbonylamino)hexanoic acid (LysN3); and 2-Amino-6-(prop-2-ynoxycarbonylamino)hexanoic acid (LysAlk), O-Sulfo-L-tyrosine (SY), 2-amino-3-[4-(carboxymethyl) phenyl]propanoic acid (CMF), L-p-hydroxy-phenyllactic acid (Ester), L-2-Amino-4-phosphonobutyric acid (PSA), O-phospho-L-serine (OPS), Acetyl-L-lysine (AcK), 4-benzoyl-1-phenylalanine (Bpa), N⁶-((2-(3-methyl-3H-diazirin-3-yl)ethoxy)carbonyl)-L-lysine (Photo-Lysine, Phk), other noncanonical amino acids known in the art, and various combinations thereof. In some embodiments, the noncanonical amino acid is selected from one or more of OmeY, AcF, AzF, OPG, AzMF, and IPhe. In some embodiments, the noncanonical amino acid is selected from at least one of DOPA and BPhe. In some embodiments, the aminoacyl-tRNA synthetases can be used to incorporate noncanonical amino acids into polypeptides expressed in yeast.

The aminoacyl-tRNA synthetase in various embodiments can be derived from a WT Escherichia coli tyrosyl-tRNA synthetase or from a WT Escherichia co/i leucyl-tRNA synthetase. In some embodiments, the aminoacyl-tRNA synthetase polypeptide is a tyrosyl-tRNA synthetase. In some embodiments, the aminoacyl-tRNA synthetase is a leucyl-tRNA synthetase. The aminoacyl-tRNA synthetase can include about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 altered amino acids relative to a corresponding reference or WT sequence. In some embodiments, the aminoacyl-tRNA synthetase is an aminoacyl-tRNA synthetase listed in any one or more of Tables 6, 11, 8-10, and 19-24 or comprising one or more alterations or combinations of alterations listed in the tables.

An aminoacyl-tRNA synthetase polypeptide is characterized by measuring a relative readthrough efficiency (RRE) and/or a maximum misincorporation frequency (MMF) of the aminoacyl-tRNA synthetase. In some embodiments, an aminoacyl-tRNA synthetase encodes a noncanonical amino acid with a relative readthrough efficiency of about or at least about 0.001, 0.002, 0.003, 0.004, 0.005, 0.006, 0.007, 0.008, 0.009, 0.01, 0.02, 0.03, 0.04 0.05, 0.06, 0.07, 0.08, 0.09, 0.1, 0.11, 0.12, 0.13, 0.14, 0.15, 0.16, 0.17, 0.18, 0.19, 0.2, 0.25, 0.3, 0.35, 0.4, 0.45, or 0.5. In some embodiments, an aminoacyl-tRNA synthetase encodes a noncanonical amino acid with a maximum misincorporation frequency of about or less than about 2.5, 2.4, 2.3, 2.2, 2.1, 2, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, or 0.01. In some embodiments, the relative readthrough efficiency (RRE) and/or the maximum misincorporation frequency (MMF) is measured with respect to encoding one or more particular noncanonical amino acids listed herein.

In some embodiments, the relative readthrough efficiency (RRE) and/or the maximum misincorporation frequency (MMF) is measured at a particular concentration of the noncanonical amino acid(s), where the concentration of the noncanonical amino acid in various embodiments is about or at least about 0.05 mM, 0.1 mM, 0.15 mM, 0.20 mM, 0.25 mM, 0.30 mM, 0.35 mM, 0.40 mM, 0.45 mM, 0.5 mM, 0.55 mM, 0.65 mM, 0.7 mM, 0.75 mM, 0.8 mM, 0.85 mM, 0.9 mM, 0.95 mM, 1 mM, 1.1 mM, 1.2 mM, 1.3 mM, 1.4 mM, 1.5 mM, 1.6 mM, 1.7 mM, 1.8 mM, 1.9 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, or 20 mM.

Methods for calculating relative readthrough efficiency (RRE) and maximum misincorporation frequency (MMF) are known and are disclosed, for example, in Potts, K. A., Stieglitz, J. T., Lei, M., Van Deventer, J. A. (2020) Reporter system architecture affects measurements of noncanonical amino acid incorporation efficiency and fidelity, Mol. Syst. Des. Eng. 5, 573-588 and Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269.

Maximum misincorporation frequency quantifies the maximum frequency with which a stop codon is read through aberrantly with a canonical amino acid instead of a noncanonical amino acid of interest (typical range of 0 to 1, with highest fidelity at a value of 0). Relative readthrough efficiency quantified the readthrough of a stop codon in comparison to the readthrough of a cognate codon (typical range of 0 to 1, with readthrough efficiency equaling the readthrough of a cognate codon at a value of 1).

In some embodiments, the aminoacyl-tRNA synthetase has specificity or polyspecificity for 1, 2, 3, 4, 5, 6, 7, 8, 9, 10, 11, 12, 13, 14, 15, 16, 17, 18, 19, 20, 25, 30, 35, 40, 45, 50, or 100 noncanonical amino acids. In some embodiments, the aminoacyl-tRNA synthetase has specificity or polyspecificity for one or more noncanonical amino acids selected from OmeY, AcF, AzF, OPG, AzMF, and IPhe.

By “orthogonal” is meant a molecule that functions with endogenous components of a cell with reduced efficiency as compared to a corresponding molecule that is endogenous to the cell or translation system, or that fails to function with endogenous components of the cell. In the context of tRNAs and aminoacyl-tRNA synthetases, orthogonal refers to an inability or reduced efficiency, e.g., less than 20% efficiency, less than 10% efficiency, less than 5% efficiency, or less than 1% efficiency, of an orthogonal tRNA to function with an endogenous tRNA synthetase compared to the ability of an endogenous tRNA to function with the endogenous tRNA synthetase; or of an orthogonal aminoacyl-tRNA synthetase to function with an endogenous tRNA compared to the ability of an endogenous tRNA synthetase to function with the endogenous tRNA. The orthogonal molecule in various embodiments lacks a functionally normal endogenous complementary molecule in the cell. For example, an orthogonal tRNA in a cell is aminoacylated by any endogenous tRNA synthetase (RS) of the cell with reduced or even undetectable or zero efficiency, when compared to aminoacylation of an endogenous tRNA by the endogenous RS. In another example, an orthogonal RS aminoacylates any endogenous tRNA in a cell of interest with reduced or even undetectable or zero efficiency, as compared to aminoacylation of the endogenous tRNA by an endogenous RS. A second orthogonal molecule can be introduced into the cell that functions with the first orthogonal molecule. For example, an orthogonal tRNA/RS pair includes introduced complementary components that function together in the cell with an efficiency (e.g., 0.01% efficiency, 0.02% efficiency, 0.03% efficiency, 0.04% efficiency, 0.05% efficiency, 0.06% efficiency, 0.07% efficiency, 0.08% efficiency, 0.09% efficiency, 0.1% efficiency, 0.2% efficiency, 0.3% efficiency, 0.4% efficiency, 0.5% efficiency, 0.6% efficiency, 0.7% efficiency, 0.8% efficiency, 0.9% efficiency, 1% efficiency, 2% efficiency, 3% efficiency, 4% efficiency, 5% efficiency, 6% efficiency, 7% efficiency, 8% efficiency, 9% efficiency, 10% efficiency, 15% efficiency, 20% efficiency, 25% efficiency, 30% efficiency, 35% efficiency, 40% efficiency, 45% efficiency, 50% efficiency, 60% efficiency, 70% efficiency, 75% efficiency, 80% efficiency, 90% efficiency, 95% efficiency, or 99% or more efficiency) as compared to that of a control, e.g., a corresponding tRNA/RS endogenous pair.

### Polypeptide Expression

In general, polypeptides (e.g., an aminoacyl-tRNA synthetase) of the invention may be produced by transformation of a suitable host cell (e.g., mammalian cell, bacterial cell, yeast cell, insect cell) with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, cells from other species of yeast, insect cells, or mammalian cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof. Once the recombinant polypeptide of the invention, or a polypeptide produced according to the methods of the present invention and containing a noncanonical amino acid, is expressed, it can be isolated, e.g., using affinity chromatography. In embodiments, the polypeptide of the invention is not isolated from a cell. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods familiar to one of skill in the art.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). These general techniques of polypeptide expression and purification can also be used to produce and isolate useful peptide fragments or analogs.

A cell can be engineered or mutated to utilize noncanonical amino acids. For example, a cell (e.g., E. coli, yeast, other prokaryotic cells, or mammalian cells) can be genomically recoded to facilitate incorporation of noncanonical amino acids (see, e.g., Liu C C, Schultz P G. Adding new chemistries to the genetic code. Annu Rev Biochem 2010, 79:413-444; Chin J W. Expanding and reprogramming the genetic code. Nature 2017, 550:53-60; and Lajoie M J et al. Genomically recoded organisms expand biological functions. Science 2013, 342:357-360). In some instances, it can be advantageous to introduce mutations to a release factor polynucleotide or polypeptide in a cell to help facilitate utilization of noncanonical amino acids (see, e.g., Chin, “Reprogramming the genetic code”, EMBO J., 30:2312-2324 (2011)).

Synthetic Yeast 2.0 is an example of engineering a cell to better utilize noncanonical amino acids. For example, in the Sc2.0 project, TAG stop codons are recoded to TAA to allow insertion of noncanonical amino acids (Jones, S. SCRaMbLE does the yeast genome shuffle. Nat Biotechnol 36, 503 (2018). doi.org/10.1038/nbt.4164).

### Screening and Expression of Polypeptides Containing Noncanonical Amino Acids

The aminoacyl-tRNA synthetases of the present disclosure may be used for the incorporation of noncanonical amino acids into a polypeptide sequence in vivo or ex vivo. The noncanonical amino acids may be incorporated anywhere in the polypeptide sequence. The noncanonical amino acids may be incorporated into the polypeptide to confer desired characteristics to the polypeptide (e.g., increased stability at high or low temperature or pH, increased activity, etc.).

Incorporation of a noncanonical amino acid into the polypeptide sequence can be achieved by inserting a codon specific for the noncanonical amino acid (e.g., an amber codon) in the polynucleotide encoding the polypeptide. In some embodiments, a single noncanonical amino acid is incorporated into the polypeptide. The noncanonical amino acid in various embodiments can be any one of the noncanonical amino acids discussed herein.

In some embodiments, noncanonical amino acids may be incorporated into the polypeptide by using either a nonsense suppressor or a frame-shift suppressor tRNA in response to amber or four-base codons, respectively (See Bain et al., J Am. Chem. Soc. 111: 8013, 1989; Noren et al., Science 244: 182, 1989; Furter, Protein Sci. 7: 419, 1998; Wang et al., Proc. Natl. Acad. Sci. U.S.A., 100: 56, 2003; Hohsaka et al., FEBS Lett. 344: 171: 1994; Kowal and Oliver, Nucleic Acids Res. 25: 4685, 1997. All incorporated herein by reference). Such methods insert noncanonical amino acids at codon positions that will normally terminate wild-type peptide synthesis (e.g. a stop codon or a frame-shift mutation).

An exemplary method for producing a polypeptide comprising a noncanonical amino acid involves: a) transforming a host cell (e.g., mammalian cell, bacterial cell, yeast cell, insect cell) with i) a vector containing a polynucleotide sequence encoding an aminoacyl-tRNA synthetase described herein capable of charging a tRNA with a noncanonical amino acid(s) of interest, ii) a vector containing a polynucleotide sequence encoding the polypeptide of interest, iii) a vector encoding a suppressor tRNA (e.g., tRNA_{CUA}) that can be charged by the aminoacyl-tRNA synthetase with a noncanonical amino acid; b) growing the host-vector system in a medium comprising the noncanonical amino acid(s) to be incorporated into the polypeptide sequence under conditions where the host vector system overexpresses the aminoacyl-tRNA synthetase and the suppressor tRNA, c) inducing expression of the polypeptide of interest, thereby incorporating the noncanonical amino acid(s) into the polypeptide. The method can further comprise isolating the polypeptide of interest using methods known in the art, optionally those described herein. In various embodiments, the polypeptide of interest can be fused to tags to assist in downstream isolation (e.g., a His-tag or a FLAG tag). In some embodiments, the polypeptide of interest is fused to a secretion signal and/or is encoded by a secretion plasmid. In various embodiments, the suppressor tRNA is an orthogonal tRNA (i.e., a tRNA not aminoacylated by an aminoacyl-tRNA synthetase naturally expressed by the host cell).

For in vitro use, one or more aminoacyl-tRNA synthetases of the present disclosure can be recombinantly produced and supplied to an in vitro translation systems (e.g., the commercially available Wheat Germ Lysate-based PROTEINscript-PRO™, Ambion's E. coli system for coupled in vitro transcription/translation; or the rabbit reticulocyte lysate-based Retic Lysate IVT™ Kit from Ambion). Optionally, the in vitro translation system can be selectively depleted of one or more natural canonical aminoacyl-tRNA synthetases (by, for example, immunodepletion using immobilized antibodies against natural aminoacyl-tRNA synthetases) and/or natural amino acids so that enhanced incorporation of the analog can be achieved.

In some embodiments, the methods of the present disclosure involving preparing a library of polynucleotide sequences encoding the polypeptide sequence of interest with codons for incorporation of the noncanonical amino acid sequence at various, optionally random, sites. The methods can further include expressing each of the polynucleotide sequences under conditions, such as those described above, allowing for incorporation of the noncanonical amino acid(s) into the encoded polypeptide sequences and subsequently screening the expressed polypeptides for characteristics of interest. Non-limiting examples of methods for screening polypeptides expressed in yeast and for expression of polypeptides in yeast are described in Boder and Wittrup, “Yeast surface display for screening combinatorial polypeptide libraries,” Nature Biotechnology, 15:553-557 (1997). Further non-limiting examples of methods for rapid screening and characterization of polypeptides expressed in yeast are provided in Van Deventer, J. A.; Kelly, R. L. et al. Protein Eng Des Sel 2015, 28, 317; Van Deventer, J. A. et al, Protein Eng Des Sel 2016, 29, 485-94; Stieglitz, J. T., Kehoe, H. P., Lei, M. and Van Deventer, J. A., ACS Synth Biol 2018, 7 (9), 2256-2269; and Potts, K. A., Stieglitz, J. T., Lei, M., and Van Deventer, J. A., Mol. Syst. Des. Eng. 2020. 5, 573-588.

Various host cells may be used for this method, including those of prokaryotic, yeast, mammalian, insect, or plant cells. In various embodiments, the polypeptide comprising the noncanonical amino acid(s) of interest can be expressed in vitro. Non-limiting examples of prokaryotic host cells include Escherichia coli, Thermus thermophilus, Bacillus stearothermophilus. Examples of Archaea include, e.g., Methanococcus jannaschii, Methanosarcina mazei, Methanobacterium thermoautotrophicum, Methanococcus maripaludis, Methanopyrus kandleri, Halobacterium such as Haloferax volcanii and Halobacterium species NRC-1, Archaeoglobus fulgidus, Pyrococcus furiosus, Pyrococcus horikoshii, Pyrobaculum aerophilum, Pyrococcus abyssi, Sulfolobus solfataricus, Sulfolobus tokodaii, Aeuropyrum pernix, Thermoplasma acidophilum, and Thermoplasma volcanium. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987). The host cell can be in in culture.

The polypeptide of interest can be a therapeutic protein, a diagnostic protein, an industrial enzyme, or portion thereof, and the like. In some cases, the polypeptide of interest is a growth factor, an antibody or a fragment thereof, a cytokine, a chemokine, an extracellular matrix protein, a polypeptide having an immune-modulatory function, an interleukin, an interferon, an immune-checkpoint blockade polypeptide, an antigen recognition polypeptide, a binding agent (e.g., fibronectin), or an alpha-helical peptide or ligand thereof. Examples of therapeutic, diagnostic, and other proteins that can be modified to comprise one or more noncanonical amino acids according to the methods of the present disclosure include, but are not limited to, e.g., Alpha-1 antitrypsin, Angiostatin, Antihemolytic factor, antibodies, Apolipoprotein, Apoprotein, Atrial natriuretic factor, Atrial natriuretic polypeptide, Atrial peptides, C-X-C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-1 beta, RANTES, 1309, R83915, R91733, HCC1, T58847, D31065, T64262), CD40 ligand, C-kit Ligand, Collagen, Colony stimulating factor (CSF), Complement factor 5a, Complement inhibitor, Complement receptor 1, cytokines, (e.g., epithelial Neutrophil Activating Peptide-78, GROa/MGSA, GROβ, GROγ, MIP-1α, MIP-1δ, MCP-1), Epidermal Growth Factor (EGF), Erythropoietin (“EPO”), Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog proteins (e.g., Sonic, Indian, Desert), Hemoglobin, Hepatocyte Growth Factor (HGF), Hirudin, Human serum albumin, Insulin, Insulin-like Growth Factor (IGF), interferons (e.g., IFN-α, IFN-β, IFN-γ), interleukins (e.g., IL-1, IL-2, IL-3, IL-4, IL-5, IL-6, IL-7, IL-8, IL-9, IL-10, IL-11, IL-12, etc.), cytokines, Keratinocyte Growth Factor (KGF), Lactoferrin, leukemia inhibitory factor, Luciferase, Neurturin, Neutrophil inhibitory factor (NIF), oncostatin M, Osteogenic protein, Parathyroid hormone, PD-ECSF, PDGF, peptide hormones (e.g., Human Growth Hormone), Pleiotropin, Protein A, Protein G, Pyrogenic exotoxins A, B, and C, Relaxin, Renin, SCF, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Superoxide dismutase (SOD), Toxic shock syndrome toxin (TSST-1), Thymosin alpha 1, Tissue plasminogen activator, Tumor necrosis factor beta (TNF beta), Tumor necrosis factor receptor (TNFR), Tumor necrosis factor-alpha (TNF alpha), Vascular Endothelial Growth Factor (VEGF), Urokinase and many others.

In some embodiments, the polypeptide of interest is a transcriptional modulator or a portion thereof. Example transcriptional modulators include genes and transcriptional modulator proteins that modulate cell growth, differentiation, regulation, or the like. Transcriptional modulators are found in prokaryotes, viruses, and eukaryotes, including fungi, plants, yeasts, insects, and animals, including mammals, providing a wide range of therapeutic targets. It will be appreciated that expression and transcriptional activators regulate transcription by many mechanisms, e.g., by binding to receptors, stimulating a signal transduction cascade, regulating expression of transcription factors, binding to promoters and enhancers, binding to proteins that bind to promoters and enhancers, unwinding DNA, splicing pre-mRNA, polyadenylating RNA, and degrading RNA.

In some embodiments, the polypeptide of interest is an expression activator such as cytokines, inflammatory molecules, growth factors, their receptors, and oncogene products, e.g., interleukins (e.g., IL-1, IL-2, IL-8, etc.), interferons, FGF, IGF-I, IGF-II, FGF, PDGF, TNF, TGF-α, TGF-β, EGF, KGF, SCF/c-Kit, CD40L/CD40, VLA-4/VCAM-1, ICAM-1/LFA-1, and hyalurin/CD44; signal transduction molecules and corresponding oncogene products, e.g., Mos, Ras, Raf, and Met; and transcriptional activators and suppressors, e.g., p53, Tat, Fos, Myc, Jun, Myb, Rel, and steroid hormone receptors such as those for estrogen, progesterone, testosterone, aldosterone, the LDL receptor ligand and corticosterone.

The protein of interest can be an enzyme. Examples of enzymes include, but are not limited to, e.g., amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

The polypeptide of interest can be a protein from infectious fungi, e.g., Aspergillus, Candida species; bacteria, particularly E. coli, which serves a model for pathogenic bacteria, as well as medically important bacteria such as Staphylococcus spp. (e.g., S. aureus), or Streptococcus spp. (e.g., S. pneumoniae); protozoa such as sporozoa (e.g., Plasmodium spp.), rhizopods (e.g., Entamoeba spp.) and flagellates (Trypanosoma spp., Leishmania spp., Trichomonas spp., Giardia spp., etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), (−) RNA viruses (e.g., Rhabdoviruses, e.g., VSV; Paramyxoviruses, e.g., RSV; Orthomyxoviruses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

The polypeptide of interest can be an agriculturally related protein. Non-limiting examples of agriculturally related proteins include insect resistance proteins (e.g., the Cry proteins), starch and lipid production enzymes, plant and insect toxins, toxin-resistance proteins, Mycotoxin detoxification proteins, plant growth enzymes (e.g., Ribulose 1,5-Bisphosphate Carboxylase/Oxygenase, “RUBISCO”), lipoxygenase (LOX), and Phosphoenolpyruvate (PEP) carboxylase.

### Pharmaceutical Compositions

The present invention contemplates pharmaceutical preparations comprising polypeptides produced by the methods of the present disclosure, together with pharmaceutically acceptable carriers. Polypeptides of the invention may be administered as part of a pharmaceutical composition. The compositions should be sterile and contain a therapeutically effective amount of the polypeptides in a unit of weight or volume suitable for administration to a subject.

Pharmaceutical compositions of the invention to be used for therapeutic administration should be sterile. Sterility is readily accomplished by filtration through sterile filtration membranes (e.g., 0.2 mm membranes), by gamma irradiation, or any other suitable means known to those skilled in the art. Therapeutic polypeptide compositions generally are placed into a container having a sterile access port, for example, an intravenous solution bag or vial having a stopper pierceable by a hypodermic injection needle. These compositions ordinarily will be stored in unit or multi-dose containers, for example, sealed ampoules or vials, as an aqueous solution or as a lyophilized formulation for reconstitution. A composition for infusion can be prepared by reconstituting the lyophilized material using sterile Water-for-Injection (WFI).

The polypeptides may be combined, optionally, with a pharmaceutically acceptable excipient. The term “pharmaceutically-acceptable excipient” as used herein means one or more compatible solid or liquid filler, diluents or encapsulating substances that are suitable for administration into a human. The term “carrier” denotes an organic or inorganic ingredient, natural or synthetic, with which the active ingredient is combined to facilitate administration. The components of the pharmaceutical compositions also are capable of being co-mingled with the molecules of the present invention, and with each other, in a manner such that there is no interaction that would substantially impair the desired pharmaceutical efficacy.

Polypeptides of the present invention can be contained in a pharmaceutically acceptable excipient. The excipient preferably contains minor amounts of additives such as substances that enhance isotonicity and chemical stability. Such materials are non-toxic to recipients at the dosages and concentrations employed, and include buffers such as phosphate, citrate, succinate, acetate, lactate, tartrate, and other organic acids or their salts; tris-hydroxymethylaminomethane (TRIS), bicarbonate, carbonate, and other organic bases and their salts; antioxidants, such as ascorbic acid; low molecular weight (for example, less than about ten residues) polypeptides, e.g., polyarginine, polylysine, polyglutamate and polyaspartate; proteins, such as serum albumin, gelatin, or immunoglobulins; hydrophilic polymers, such as polyvinylpyrrolidone (PVP), polypropylene glycols (PPGs), and polyethylene glycols (PEGs); amino acids, such as glycine, glutamic acid, aspartic acid, histidine, lysine, or arginine; monosaccharides, disaccharides, and other carbohydrates including cellulose or its derivatives, glucose, mannose, sucrose, dextrins or sulfated carbohydrate derivatives, such as heparin, chondroitin sulfate or dextran sulfate; polyvalent metal ions, such as divalent metal ions including calcium ions, magnesium ions and manganese ions; chelating agents, such as ethylenediamine tetraacetic acid (EDTA); sugar alcohols, such as mannitol or sorbitol; counterions, such as sodium or ammonium; and/or nonionic surfactants, such as polysorbates or poloxamers. Other additives may be included, such as stabilizers, anti-microbials, inert gases, fluid and nutrient replenishers (i.e., Ringer's dextrose), electrolyte replenishers, and the like, which can be present in conventional amounts.

The compositions can be administered in effective amounts. The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result.

A variety of administration routes are available. The methods of the invention, generally speaking, may be practiced using any mode of administration that is medically acceptable, meaning any mode that produces effective levels of the active compounds without causing clinically unacceptable adverse effects. In one embodiment, a composition of the invention comprising a polypeptide comprising a noncanonical amino acid is administered by any of various modes including inhalation, oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes. A particular method of administration involves coating, embedding or derivatizing fibers, such as collagen fibers, protein polymers, etc. with therapeutic proteins. Other useful approaches are described in Otto, D. et al., J. Neurosci. Res. 22: 83-91 and in Otto, D. and Unsicker, K. J. Neurosci. 10: 1912-1921.

The term “parenteral” includes subcutaneous, intrathecal, intravenous, intramuscular, intraperitoneal, or infusion. Intravenous or intramuscular routes are not particularly suitable for long-term therapy and prophylaxis. They could, however, be preferred in emergency situations. Compositions comprising polypeptides produced by the methods of the present disclosure can be added to a physiological fluid such as blood or synovial fluid. For CNS administration, a variety of techniques are available for promoting transfer of the therapeutic across the blood brain barrier including disruption by surgery or injection, drugs which transiently open adhesion contact between the CNS vasculature endothelial cells, and compounds that facilitate translocation through such cells. Oral administration can be preferred for prophylactic treatment because of the convenience to the patient as well as the dosing schedule.

Pharmaceutical compositions of the invention can optionally further contain one or more additional proteins as desired, including plasma proteins, proteases, and other biological material, so long as it does not cause adverse effects upon administration to a subject. Suitable proteins or biological material may be obtained from human or mammalian plasma by any of the purification methods known and available to those skilled in the art; from supernatants, extracts, or lysates of recombinant tissue culture, viruses, yeast, bacteria, or the like that contain a gene that expresses a human or mammalian plasma protein which has been introduced according to standard recombinant DNA techniques; or from the fluids (e.g., blood, milk, lymph, urine or the like) or transgenic animals that contain a gene that expresses a human plasma protein which has been introduced according to standard transgenic techniques.

Pharmaceutical compositions of the invention can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions of the invention can also contain one or more osmotic modulating agents, i.e., a compound that modulates the osmotic properties (e.g., tonicity, osmolality and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

Compositions comprising polypeptides comprising noncanonical amino acids and produced by methods of the present disclosure can contain multivalent metal ions, such as calcium ions, magnesium ions and/or manganese ions. Any multivalent metal ion that helps stabilize the polypeptide composition and that will not adversely affect recipient individuals may be used. The skilled artisan, based on these two criteria, can determine suitable metal ions empirically and suitable sources of such metal ions are known, and include inorganic and organic salts.

Pharmaceutical compositions of the invention can also be a non-aqueous liquid formulation. Any suitable non-aqueous liquid may be employed, provided that it provides stability to the active agents (s) contained therein. Preferably, the non-aqueous liquid is a hydrophilic liquid. Illustrative examples of suitable non-aqueous liquids include: glycerol; dimethyl sulfoxide (DMSO); polydimethylsiloxane (PMS); ethylene glycols, such as ethylene glycol, diethylene glycol, triethylene glycol, polyethylene glycol (“PEG”) 200, PEG 300, and PEG 400; and propylene glycols, such as dipropylene glycol, tripropylene glycol, polypropylene glycol (“PPG”) 425, PPG 725, PPG 1000, PPG 2000, PPG 3000 and PPG 4000. Pharmaceutical compositions of the invention can also be a mixed aqueous/non-aqueous liquid formulation. Any suitable non-aqueous liquid formulation, such as those described above, can be employed along with any aqueous liquid formulation, such as those described above, provided that the mixed aqueous/non-aqueous liquid formulation provides stability to the polypeptide contained therein. Preferably, the non-aqueous liquid in such a formulation is a hydrophilic liquid. Illustrative examples of suitable non-aqueous liquids include: glycerol; DMSO; PMS; ethylene glycols, such as PEG 200, PEG 300, and PEG 400; and propylene glycols, such as PPG 425, PPG 725, PPG 1000, PPG 2000, PPG 3000 and PPG 4000. Suitable stable formulations can permit storage of the active agents in a frozen or an unfrozen liquid state. Stable liquid formulations can be stored at a temperature of at least −70° C., but can also be stored at higher temperatures of at least 0° C., or between about 0.1° C. and about 42° C., depending on the properties of the composition. It is generally known to the skilled artisan that proteins and polypeptides are sensitive to changes in pH, temperature, and a multiplicity of other factors that may affect therapeutic efficacy.

In certain embodiments a desirable route of administration can be by pulmonary aerosol. Techniques for preparing aerosol delivery systems containing polypeptides are well known to those of skill in the art. Generally, such systems should utilize components that will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily modify the various parameters and conditions for producing polypeptide aerosols without resorting to undue experimentation.

Other delivery systems can include time-release, delayed release or sustained release delivery systems. Such systems can avoid repeated administrations of polypeptides, increasing convenience to the subject and the physician. Many types of release delivery systems are available and known to those of ordinary skill in the art. They include polymer base systems such as polylactides (U.S. Pat. No. 3,773,919; European Patent No. 58,481), poly(lactide-glycolide), copolyoxalates, polycaprolactones, polyesteramides, polyorthoesters, polyhydroxybutyric acids, such as poly-D-(−)-3-hydroxybutyric acid (European Patent No. 133, 988), copolymers of L-glutamic acid and gamma-ethyl-L-glutamate (Sidman, K. R. et al., Biopolymers 22: 547-556), poly (2-hydroxyethyl methacrylate) or ethylene vinyl acetate (Langer, R. et al., J. Biomed. Mater. Res. 15:267-277; Langer, R. Chem. Tech. 12:98-105), and polyanhydrides.

Other examples of sustained-release compositions include semi-permeable polymer matrices in the form of shaped articles, e.g., films, or microcapsules. Delivery systems also include non-polymer systems that are: lipids including sterols such as cholesterol, cholesterol esters and fatty acids or neutral fats such as mono-di- and tri-glycerides; hydrogel release systems such as biologically-derived bioresorbable hydrogel (i.e., chitin hydrogels or chitosan hydrogels); sylastic systems; peptide based systems; wax coatings; compressed tablets using conventional binders and excipients; partially fused implants; and the like.

Another type of delivery system that can be used with the methods and compositions of the invention is a colloidal dispersion system. Colloidal dispersion systems include lipid-based systems including oil-in-water emulsions, micelles, mixed micelles, and liposomes. Liposomes are artificial membrane vessels, which are useful as a delivery vector in vivo or in vitro. Large unilamellar vessels (LUV), which range in size from 0.2-4.0 μm, can encapsulate large macromolecules within the aqueous interior and be delivered to cells in a biologically active form (Fraley, R., and Papahadjopoulos, D., Trends Biochem. Sci. 6: 77-80).

Liposomes can be targeted to a particular tissue by coupling the liposome to a specific ligand such as a monoclonal antibody, sugar, glycolipid, or protein. Liposomes are commercially available from Gibco BRL, for example, as LIPOFECTIN™ and LIPOFECTACE™, which are formed of cationic lipids such as N-[1-(2, 3 dioleyloxy)-propyl]-N, N, N-trimethylammonium chloride (DOTMA) and dimethyl dioctadecylammonium bromide (DDAB). Methods for making liposomes are well known in the art and have been described in many publications, for example, in DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88, 046; EP 143,949; EP 142,641; Japanese Pat. Appl. 83-118008; U.S. Pat. Nos. 4,485,045 and 4,544,545; and EP 102,324. Liposomes also have been reviewed by Gregoriadis, G., Trends Biotechnol., 3: 235-241).

Another type of vehicle is a biocompatible microparticle or implant that is suitable for implantation into the mammalian recipient. Exemplary bioerodible implants that are useful in accordance with this method are described in PCT International application no. PCT/US/03307 (Publication No. WO 95/24929, entitled “Polymeric Gene Delivery System”). PCT/US/03307 describes biocompatible, preferably biodegradable polymeric matrices for containing an exogenous gene under the control of an appropriate promoter. The polymeric matrices can be used to achieve sustained release of the exogenous gene or gene product in the subject.

The polymeric matrix preferably is in the form of a microparticle such as a microsphere (wherein an agent is dispersed throughout a solid polymeric matrix) or a microcapsule (wherein an agent is stored in the core of a polymeric shell). Microcapsules of the foregoing polymers containing drugs are described in, for example, U.S. Pat. No. 5,075,109. Other forms of the polymeric matrix for containing an agent include films, coatings, gels, implants, and stents. The size and composition of the polymeric matrix device is selected to result in favorable release kinetics in the tissue into which the matrix is introduced. The size of the polymeric matrix further is selected according to the method of delivery that is to be used. Preferably, when an aerosol route is used the polymeric matrix and polypeptides are encompassed in a surfactant vehicle. The polymeric matrix composition can be selected to have both favorable degradation rates and also to be formed of a material, which is a bioadhesive, to further increase the effectiveness of transfer. The matrix composition also can be selected not to degrade, but rather to release by diffusion over an extended period of time. The delivery system can also be a biocompatible microsphere that is suitable for local, site-specific delivery. Such microspheres are disclosed in Chickering, D. E., et al., Biotechnol. Bioeng., 52: 96-101; Mathiowitz, E., et al., Nature 386: 410-414.

Both non-biodegradable and biodegradable polymeric matrices can be used to deliver the polypeptide compositions of the invention to the subject. Such polymers may be natural or synthetic polymers. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, and polymers of lactic acid and glycolic acid, polyanhydrides, poly(ortho)esters, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

### Kits

The invention provides kits for the incorporation of noncanonical amino acids into a polypeptide of interest. The invention also provides for kits comprising a pharmaceutical composition comprising a polypeptide comprising a noncanonical amino acid produced by the methods of the present invention for administration to a subject. The agents described herein may, in some embodiments, be assembled into pharmaceutical or diagnostic or research kits to facilitate their use in therapeutic, diagnostic or research applications. In certain embodiments agents in a kit may be suitable for use in the production of polypeptides comprising noncanonical amino acids and/or for use in screens for polypeptides comprising noncanonical amino acids and having improved characteristics. Kits for research purposes may contain the components in appropriate concentrations or quantities for running various experiments.

Kits may include ampules or aliquots of compositions of the present invention. Kits may also contain devices necessary for use of components of the kit in various methods of the present disclosure. In some embodiments, the kit comprises a sterile container which contains a composition (e.g., a therapeutic or prophylactic composition); such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The kit may be designed to facilitate the methods described herein. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or another suitable solvent), which may or may not be provided with the kit. In some embodiments, the kit contains one or more of the cells described herein. The kit can contain a yeast cell.

The kit may contain any one or more of the components described herein in one or more containers. As an example, in one embodiment, the kit may include instructions for mixing one or more components of the kit and/or isolating and mixing a sample. The kit may contain instructions for administering a composition of the kit to a subject. The kit may include a container housing agents described herein. The agents may be in the form of a liquid, gel or solid (powder). The agents may be prepared sterilely, packaged in syringe and shipped refrigerated. A second container may comprise other agents prepared sterilely. Alternatively the kit may include agents premixed and shipped in a syringe, vial, tube, or other container. The kit may have one or more or all of the components useful to administer the agents to a subject, such as a syringe, topical application devices, or intravenous needle tubing and bag.

The instructions contained in the kit will generally include information about the use of the compositions of the kit in the methods of the present disclosure. In other embodiments, the instructions include at least one of the following: safety information; information describing how to use components of the kit in the methods of the present disclosure; and/or references. The instructions may be printed directly on the container (when present), provided on a transportable storage medium, stored on a remote server, or provided as a label applied to the container, or as a separate sheet, pamphlet, card, or folder supplied in or with the container. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1. E. coli Aminoacyl-tRNA Synthetase (aaRS) Saturation Mutagenesis Library Design

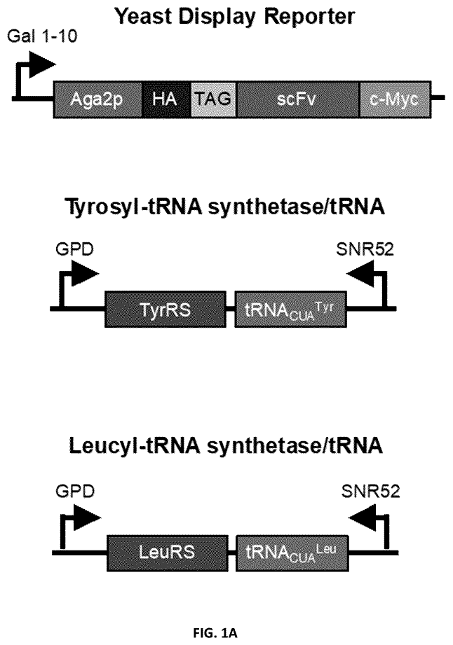

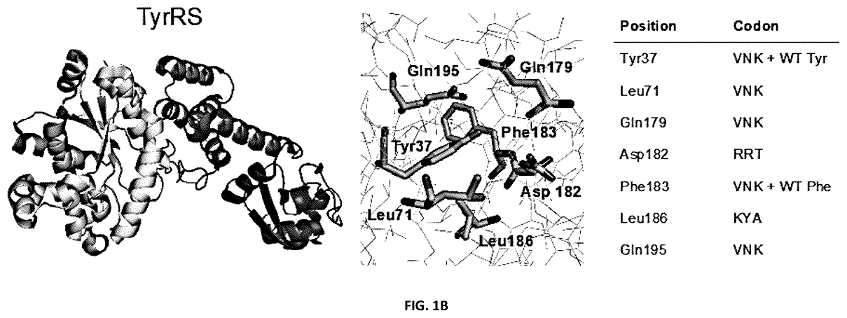

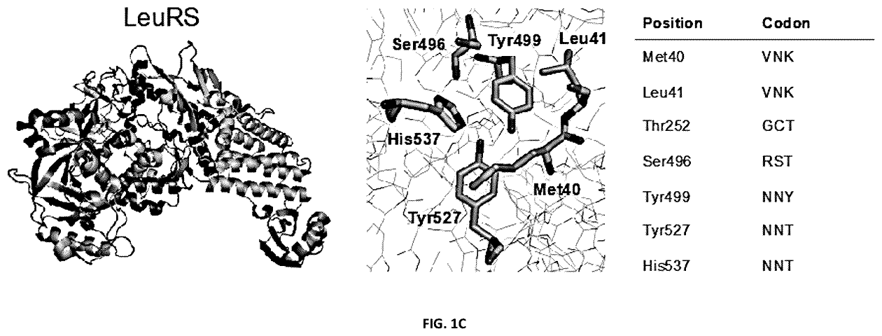

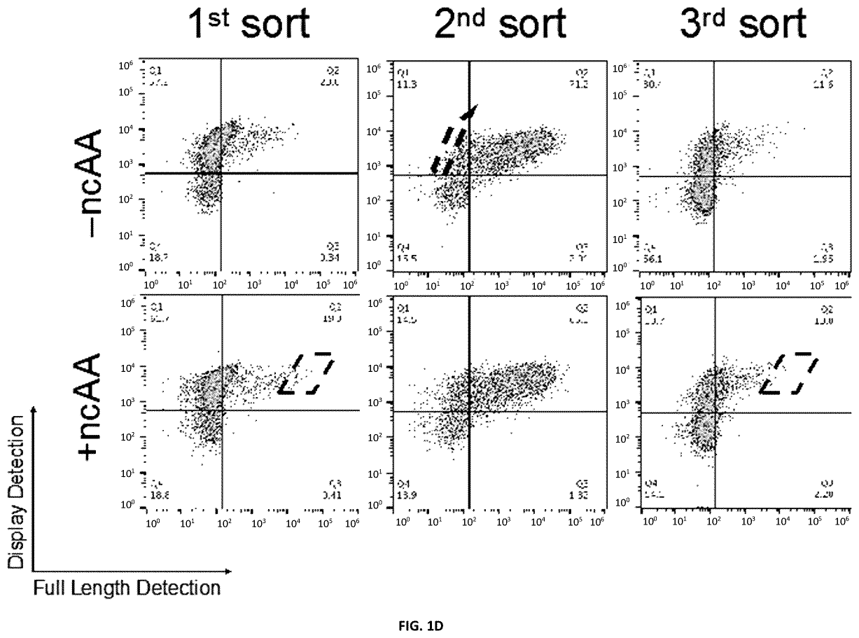

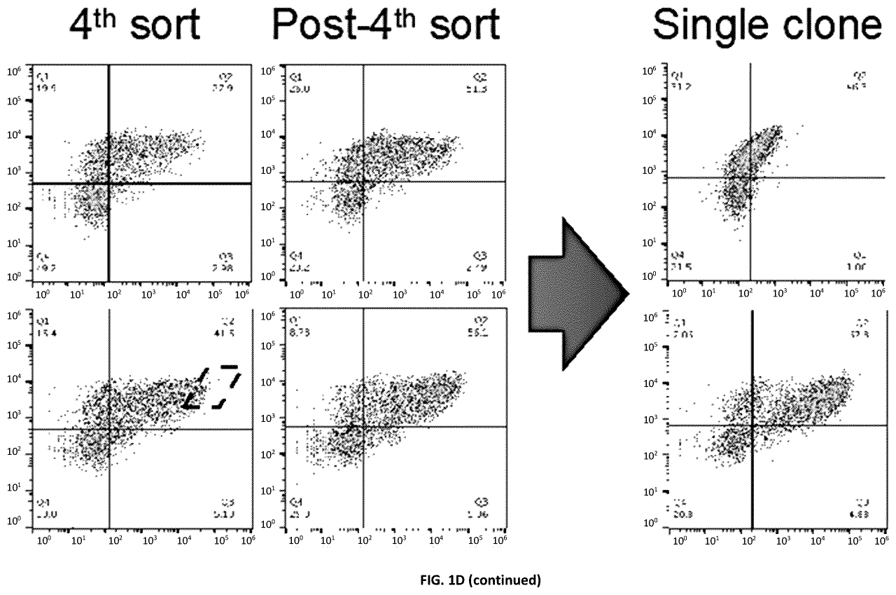

To identify aminoacyl-tRNA synthetases (aaRSs) capable of coupling versatile noncanonical amino acids (ncAAs) to tRNA_{CUA} (transfer RNA with a CUA anticodon), two saturation mutagenesis libraries were constructed of the E. coli Tyrosyl-tRNA synthetase (EcTyrRS) and the E. coli Leucyl-tRNA synthetase (EcLeuRS) (FIGS. 1A and 1B). Both EcTyrRS (E. coli Tyrosyl-tRNA synthetase) and E. coli Leucyl-tRNA synthetase (EcLeuRS) are orthogonal to the native translation machinery present in S. cerevisiae, and they can be used to encode noncanonical amino acids (ncAAs) in response to the amber stop codon. Several residues in the binding pocket of each aminoacyl-tRNA synthetase (aaRS) were chosen for mutation. In EcTyrRS (E. coli Tyrosyl-tRNA synthetase), degenerate codons at positions Y37, L71, Q179, D182, F183, L186, and Q195 were designed to reduce the size of residues in the active site that were expected to interact with the noncanonical amino acid (ncAA) as it is charged to the cognate tRNA_{CUA} (transfer RNA with a CUA anticodon) (FIG. 1B). Similarly in E. coli Leucyl-tRNA synthetase (EcLeuRS), positions M40, L41, S496, Y499, Y527, and H537 were randomized to smaller residues with an additional T252A mutation in the editing domain known to reduce the instances of leucine being charged to the tRNA^{Leu} (FIG. 1C). The permitted residues in each aminoacyl-tRNA synthetase (aaRS) active site were chosen to allow unique noncanonical amino acids (ncAAs) with bulkier or longer side chains to access the active sites and be charged to the tRNA_{CUA} (transfer RNA with a CUA anticodon), and subsequently improve noncanonical amino acid (ncAA) incorporation at a TAG (amber) codon in a reporter protein.

### Example 2. E. coli Aminoacyl-tRNA Synthetase (aaRS) Saturation Mutagenesis Library Construction

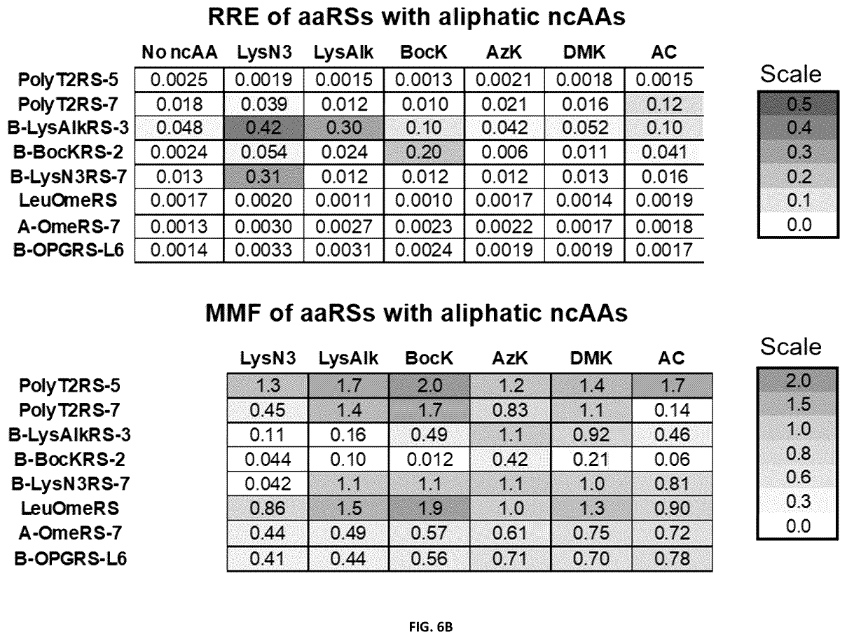

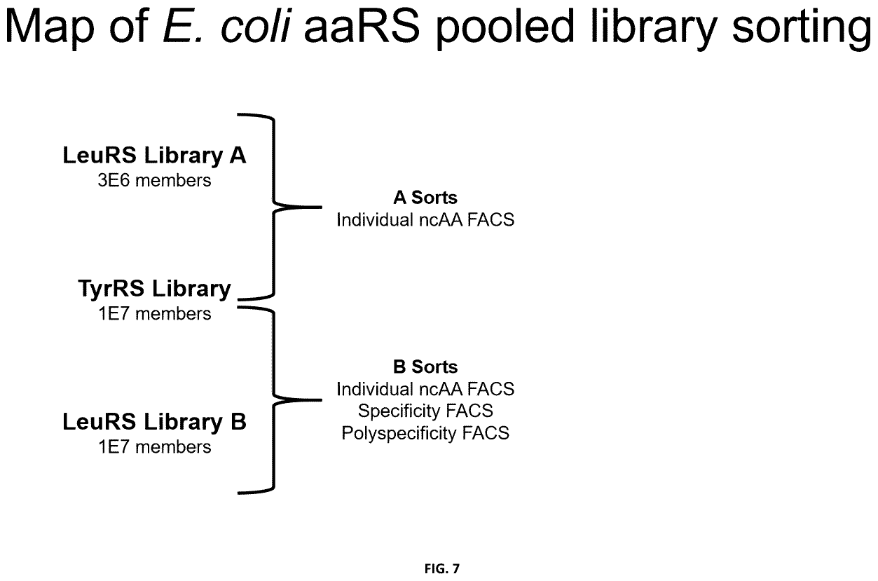

A yeast display system was used to report on noncanonical amino acid (ncAA) incorporation during screens with fluorescence-activated cell sorting (FACS). The reporter supported highly stringent screening conditions and both positive and negative screens in the same library populations. Additionally, with a series of controls and set conditions, a quantitative measurement of noncanonical amino acid (ncAA) incorporation for individual aminoacyl-tRNA synthetase (aaRS) variants identified from screening were reported for comparison of aminoacyl-tRNA synthetase (aaRS) activity against wildtype protein translation. Both libraries were constructed in S. cerevisiae RJY100 using homologous recombination and then evaluated for aminoacyl-tRNA synthetase (aaRS) activity and sequence diversity (Tables 1-5). The theoretical diversity of the EcTyrRS (E. coli Tyrosyl-tRNA synthetase) library was 1.3×10⁸, and the actual number of transformants was calculated to be 1×10⁷ and all 10 random clones that were sequenced were unique (Table 12). The theoretical diversity of the E. coli Leucyl-tRNA synthetase (EcLeuRS) library was 1.9×10⁷, and the calculated number of transformants was 3×10⁶. Sequence characterization of the E. coli Leucyl-tRNA synthetase (EcLeuRS) library revealed that out of ten clones, the last two positions chosen for mutation (Y527 and H537) were disproportionately wild type residues (Table 13). To correct the lack of mutations in those active site residues, a second library was constructed with modified primers and was determined to contain 1×10⁷ transformants (Table 5). Sequence characterization of nine clones from the reconstructed E. coli Leucyl-tRNA synthetase (EcLeuRS) library revealed that all active site positions contained expected mutations (Table 14). In order to facilitate screening, both the E. coli Tyrosyl-tRNA synthetase (EcTyrRS) and E. coli Leucyl-tRNA synthetase (EcLeuRS) libraries were pooled prior to sorting (FIG. 7). To distinguish between the first E. coli Leucyl-tRNA synthetase (EcLeuRS) library and the reconstructed E. coli Leucyl-tRNA synthetase (EcLeuRS) library the original library with predominantly WT residues at positions Y527 and H537 was named Library A and the reconstructed library was named Library B. Pooled libraries were called A or B depending on which E. coli Leucyl-tRNA synthetase (EcLeuRS) library was pooled with the EcTyrRS (E. coli Tyrosyl-tRNA synthetase) library prior to screening.

TABLE 1
Tyrosyl-tRNA synthetase (TyrRS) library theoretical diversity.

| Position | Codon | # Codons |
|---|---|---|
| Y37 | VNK + TAT | 25 |
| L71 | VNK | 24 |
| Q179 | VNK | 24 |
| D182 | RRT | 4 |
| F183 | VNK | 24 |
| L186 | KYA | 4 |
| Q195 | VNK | 24 |

| | |
|---|---|
| Theoretical Diversity | 1.3E8 |

TABLE 2
Tyrosyl-tRNA synthetase (TyrRS) library
primer design.

| | | SEQ | | SEQ |
|---|---|---|---|---|
| Posi- | | ID | | ID |
| tion | Forward | NO: | Reverse | NO: |
| Y37 | GAGCGACTGGCGCA | 6 | GCTGTCAGCGGTAG | 11 |
| | AGGCCCGATCGCGC | | GATCGAAGCCGCAM | |
| | TCVNKTGCGGCTTC | | NBGAGCGCGATCGG | |
| | GATCCTACCGCTGA | | GCCTTGCGCCAGTC | |
| | CAGC | | GCTC | |
| Y37 | GAGCGACTGGCGCA | 7 | GCTGTCAGCGGTAG | 12 |
| | AGGCCCGATCGCGC | | GATCGAAGCCGCAA | |
| | TCTATTGCGGCTTC | | TAGAGCGCGATCGG | |
| | GATCCTACCGCTGA | | GCCTTGCGCCAGTC | |
| | CAGC | | GCTC | |
| L41 | CAGCAGGCGGGCCA | 8 | GTCGCCAATCAGAC | 13 |
| | CAAGCCGGTTGCGV | | CCGTCGCGCCGCCM | |
| | NKGTAGGCGGCGCG | | NBTACCGCAACCGG | |
| | ACGGGTCTGATTGG | | CTGTGGCCCGCCTG | |
| | CGAC | | CTG | |
| Q179, | CGTTCACTGAGTTT | 9 | CAATTTGCAGCACC | 14 |
| D182, | TCCTACAACCTGTT | | ACACCGTACTGTTT | |
| F183, | GVNKGGTTATRRTV | | GTTTRMACAGGCMN | |
| L186 | NKGCCTGTKYAAAC | | BAYYATAACCMNBC | |
| | AAACAGTACGGTGT | | AACAGGTTGTAGGA | |
| | GGTGCTGCAAATTG | | AAACTCAGTGAACG | |
| Q195 | GTGCTAACAAACAG | 10 | GTTACCCCACTGGT | 15 |
| | TACGGTGTGGRGCT | | CAGAACCAACCAAT | |
| | GVNKATTGGTGGTT | | MNBCAGCACCACAC | |
| | CTGACCAGTGGGG | | CGTACTGTTTGTTA | |
| | TAAC | | GCAC | |

TABLE 3
LeuRS library theoretical diversity.

| Position | Codon | # Codons |
|---|---|---|
| M40 | VNK | 24 |
| L41 | VNK | 24 |
| T252 | GCT | 1 |
| S496 | RST | 4 |
| Y499 | NNY | 32 |
| Y527 | NNT | 16 |
| H537 | NNT | 16 |

| | |
|---|---|
| Theoretical Diversity | 1.9E7 |

TABLE 4
LeuRS Library Primers A.

| | | SEQ | | SEQ |
|---|---|---|---|---|
| Posi- | | ID | | ID |
| tion | Forward | NO: | Reverse | NO: |
| M40, | GCAAAGAGAAGTAT | 16 | CATGTGTAGTCGAC | 21 |
| L41 | TACTGCCTGTCTVN | | CAGAAGGATAGGGM | |
| | KVNKCCCTATCCTT | | NBMNBAGACAGGCA | |
| | CTGGTCGACTACAC | | GTAATACTTCTCTT | |
| | ATG | | TGC | |
| T252 | CTGACCGTTTACAC | 17 | GCTACCGCCAGGTA | 22 |
| | TACCCGCCCGGACG | | GGTACAACCCATAA | |
| | CTTTTATGGGTTGT | | AAGCGTCCGGGCGG | |
| | ACCTACCTGGCGGT | | GTAGTGTAAACGGT | |
| | AGC | | CAG | |
| S496, | GAAACCGACACTTT | 18 | CTTTGTACTGCGGG | 23 |
| Y499 | CGACACCTTTATGG | | CAAGTGTAGCGCGC | |
| | AGRSTTCCTGGNNY | | ATARNNCCAGGAAS | |
| | TATGCGCGCTACAC | | YCTCCATAAAGGTG | |
| | TTGCCCGCAGTACA | | TCGAAAGTGTCGGT | |
| | AAG | | TTC | |
| Y527 | GCGGCTAACTACTG | 19 | CATAATGGCGTGTT | 24 |
| | GCTGCCGGTGGATA | | CAATACCACCAATA | |
| | TCNNTATTGGTGGT | | NNGATATCCACCGG | |
| | ATTGAACACGCCAT | | CAGCCAGTAGTTAG | |
| | TATG | | CCGC | |
| H537 | TATTGGTGGTATTG | 20 | GTTTGTGGAAGAAG | 25 |
| | AACACGCCATTATG | | CGGAAGTAGAGCAG | |
| | NNTCTGCTCTACTT | | ANNCATAATGGCGT | |
| | CCGCTTCTTCCACA | | GTTCAATACCACCA | |
| | AAC | | ATA | |

TABLE 5
LeuRS Library Primers B.

| | | SEQ | | SEQ |
|---|---|---|---|---|
| Posi- | | ID | | ID |
| tion | Forward | NO: | Reverse | NO: |
| M40, | GCAAAGAGAAGTAT | 16 | CATGTGTAGTCGAC | 21 |
| L41 | TACTGCCTGTCTVN | | CAGAAGGATAGGGM | |
| | KVNKCCCTATCCTT | | NBMNBAGACAGGCA | |
| | CTGGTCGACTACAC | | GTAATACTTCTCTT | |
| | ATG | | TGC | |
| T252 | CTGACCGTTTACAC | 17 | GCTACCGCCAGGTA | 28 |
| | TACCCGCCCGGACG | | GGTACAACCCATAA | |
| | CTTTTATGGGTTGT | | AAGCGTCCGGGCGG | |
| | ACCTACCTGGCGGT | | GTAGTAGTAAACGG | |
| | AGC | | TCAG | |
| S496, | CAAACCGACACTTT | 26 | CTTTGTACTGCGGG | 23 |
| Y499 | CGACACCTTTATGG | | CAAGTGTAGCGCGC | |
| | AGRSTTCCTGGNNY | | ATARNNCCAGGAAS | |
| | TATGCGCGCTACAC | | YCTCCATAAAGGTG | |
| | TTGCCCGCAGTACA | | TCGAAAGTGTCGGT | |
| | AAG | | TTC | |
| Y527, | GCGGCTAACTACTG | 27 | CAGTTTGTGGAAGA | 29 |
| H537 | GCTGCCGGTTGGAT | | AGCGGAAGTAGAGC | |
| | ATCNNTATTGGTGG | | AGANNCATAATGGC | |
| | TATTGAACACGCCA | | GTGTTCAATACCAC | |
| | TTATGNNTCTGCTC | | CAATANNGATATCC | |
| | TACTTCCGCTTCTT | | ACCGGCAGCCAGTA | |
| | CCACAAACTG | | GTTAGCCGC | |

### Example 3. Screening Aminoacyl-tRNA Synthetase (aaRS) Libraries

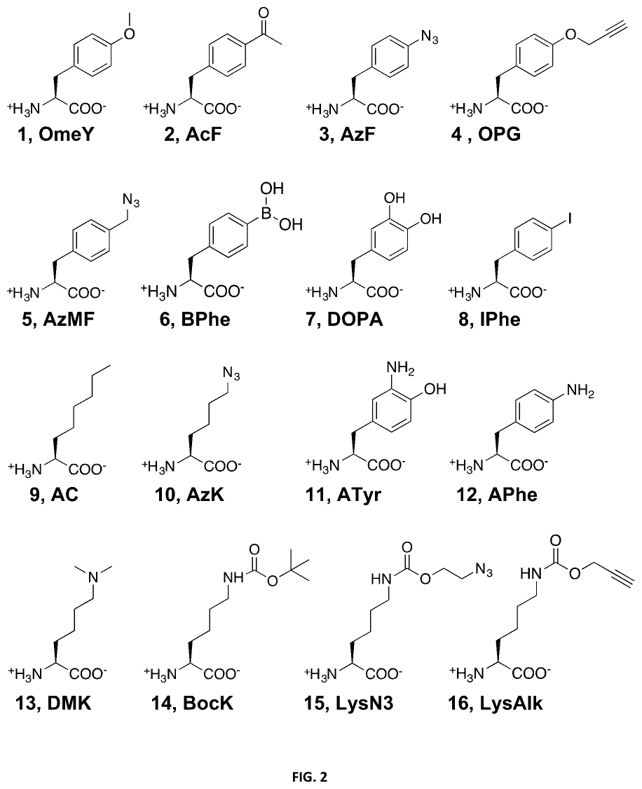

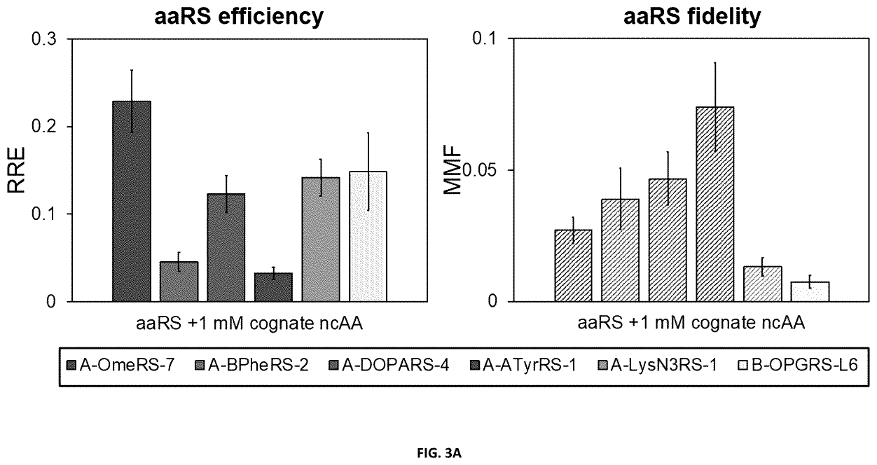

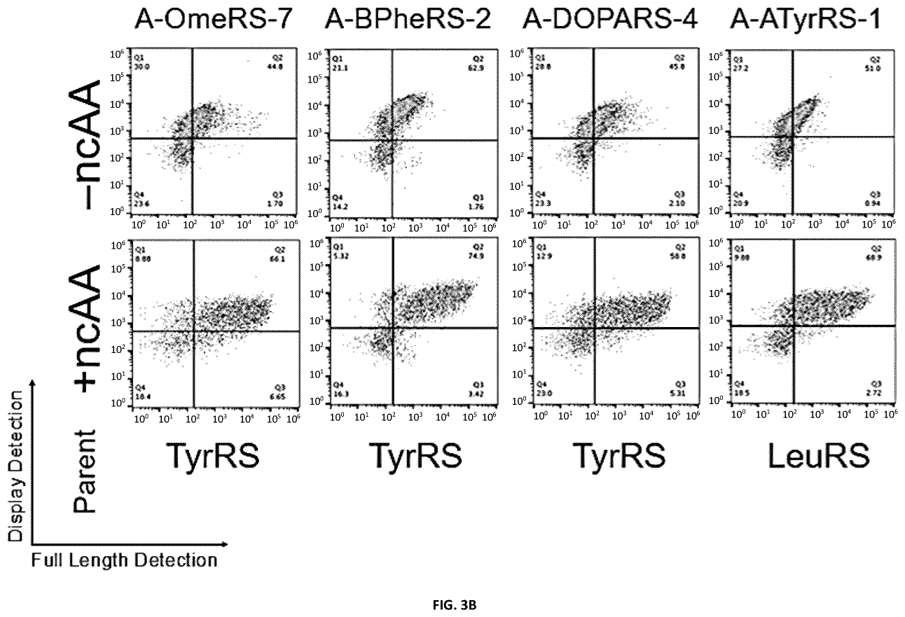

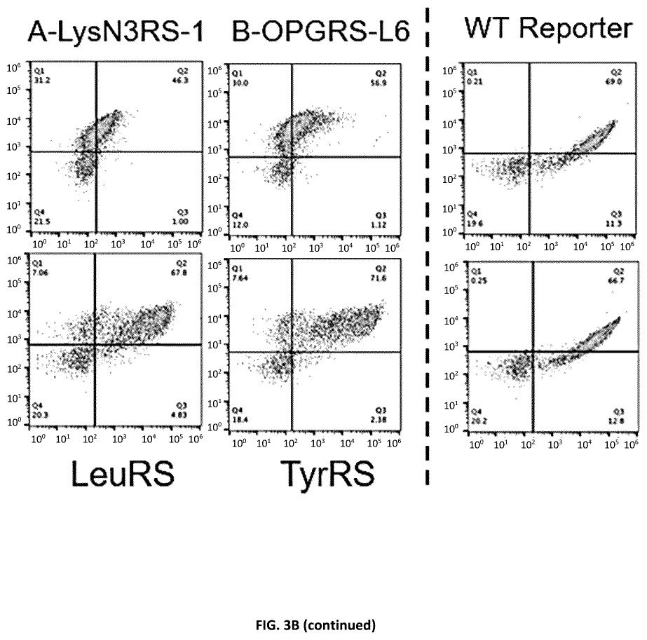

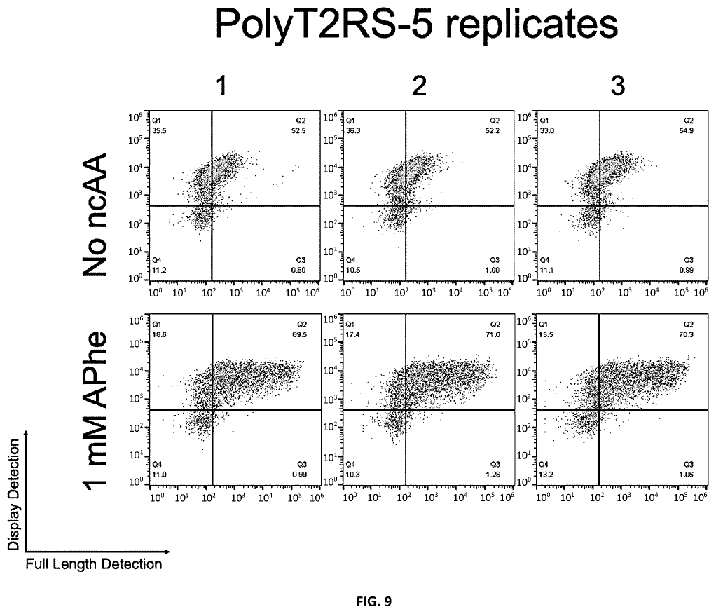

Combined EcTyrRS (E. coli Tyrosyl-tRNA synthetase) and EcLeuRS (E. coli Leucyl-tRNA synthetase) libraries were screened against several aromatic and aliphatic noncanonical amino acid (ncAA) targets to isolate aminoacyl-tRNA synthetases (aaRSs) capable of charging diverse and unique noncanonical amino acids (ncAAs) (FIG. 2). Both the original E. coli Leucyl-tRNA synthetase (EcLeuRS) library (A) and reconstructed E. coli Leucyl-tRNA synthetase (EcLeuRS) library (B) were pooled and sorted with the same EcTyrRS (E. coli Tyrosyl-tRNA synthetase) library (FIG. 7). Each noncanonical amino acid (ncAA) track was subjected to at least one negative sort where the population was induced in the absence of noncanonical amino acids (ncAAs) and cells demonstrating no amber codon suppression were recovered and at least one positive sort where the population was induced in media containing 1 mM noncanonical amino acid (ncAA) and cells demonstrating high levels of amber codon readthrough were recovered (FIG. 1D). With varying rounds of positive and negative screens, library populations with little to no misincorporation of canonical amino acids (cAAs) and moderate to high levels of noncanonical amino acid (ncAA) incorporation were enriched for several noncanonical amino acid (ncAA) targets (FIGS. 3A and 3B, and Tables 6-9). For screens with OmeY (1), one negative sort and two positive sorts yielded several clones capable of charging OmeY while discriminating against cAAs. A-OmeRS-7, had a relative readthrough efficiency (RRE) value of approximately 0.25 with a maximum misincorporation frequency (MMF) value below 0.05. For BPhe (8), two negative and three positive sorts led to isolation of an aminoacyl-tRNA synthetase (aaRS), A-BPheRS-2, that was able to charge BPhe (8) at detectable levels. Incorporation of BPhe (8) into proteins in yeast has not been achieved before, so this was an interesting result. Another noncanonical amino acid (ncAA) that has not been shown to be encoded in proteins in yeast previously is DOPA (7), for which a charging aminoacyl-tRNA synthetase (aaRS) (A-DOPARS-4) was isolated after one negative and four positive screens. Screening proceeded similarly for ATyr (11), LysN3 (15), and OPG (4). Clones A-ATyrRS-1, A-LysN3RS-1, and B-OPGRS-L6 were isolated after five positive and two negative screens, three positive and one negative screens, and two positive and one negative screens, respectively. Qualitative plots from flow cytometry indicated better incorporation of some noncanonical amino acids (ncAAs), such as APhe (FIG. 9).

TABLE 6
Sequences of aaRSs from FIG. 3.

| | Position | I37 | L71 | Q179 | G182 | M183 | A186 | Q195 |
|---|---|---|---|---|---|---|---|---|
| TyrRS | A-OmeRS-7 | L | L | Q | G | M | A | Q |
| | A-BPheRS-2 | G | T | N | S | V | E | I |
| | A-DOPARS-4 | Q | M | S | D | T | V | I |
| | B-OPGRS-L6 | V | I | Q | G | M | A | Q |

| | Position | M40 | L41 | T252 | S496 | Y499 | Y527 | H537 |
|---|---|---|---|---|---|---|---|---|
| LenRS | A-ATyrRS-1 | L | T | T | A | H | S | G |
| | A-LysN4RS | G | P | T | T | C | Y | G |

TABLE 7
Experimental details relating to sorting. Sort 1 was performed on MoFlo for aaRSs marked with an asterisk (*).

| aaRS | Sort 1 | Sort 2 | Sort 3 | Sort 4 | Sort 5 | Sort 6 | Sort 7 |
|---|---|---|---|---|---|---|---|
| A-OmeRS-7* | +1 mM OmeY | Negative | +1 mM OmeY | N/A | N/A | N/A | N/A |
| A-BPheRS-2 | +1 mM BPhe | Negative | +1 mM BPhe | Negative | +1 mM BPhe | N/A | N/A |
| A-DOPARS-4* | +1 mM DOPA | Negative | +1 mM DOPA | +1 mM DOPA | +1 mM DOPA | N/A | N/A |
| A-ATyrRS-1* | +1 mM ATyr | Negative | +1 mM ATyr | +1 mM ATyr | +1 mM ATyr | Negative | +1 mM ATyr |
| A-LysN3RS-1 | +1 mM LysN3 | Negative | +1 mM LysN3 | +1 mM LysN3 | N/A | N/A | N/A |
| B-OPGRS-L6 | +1 mM OPG | Negative | +1 mM OPG | N/A | N/A | N/A | N/A |

TABLE 8
TyrRS clone mutations.

| | TyrRS Position | | | | | | | |
|---|---|---|---|---|---|---|---|---|
| | Y37 | L71 | V72† | Q179 | D182 | F183 | L186 | Q195 |
| | Codon | | | | | | | |
| | VNK + | | | | | | | |
| | TAT | VNK | N/A | VNK | RRT | VNK | KYA | VNK |
| A-OmeRS-1 | L | V | V | Q | G | M | A | Q |
| A-OmeRS-2 | I | L | V | Q | G | M | A | Q |
| A-OmeRS-3 | G | R | V | P | S | P | L | S |
| A-OmeRS-4 | I | L | V | Q | G | M | A | Q |
| A-OmeRS-5 | I | L | V | Q | G | M | A | Q |
| A-OmeRS-6 | I | L | V | Q | G | M | A | Q |
| A-OmeRS-7 | L | L | V | Q | G | M | A | Q |
| A-OmeRS-8 | I | L | V | Q | G | M | A | Q |
| A-OmeRS-9 | I | L | V | Q | G | M | A | Q |
| A-OmeRS-10 | L | L | V | Q | G | M | A | Q |
| A-DOPARS-1 | E | L | V | Q | G | M | A | T |
| A-DOPARS-2 | L | L | V | G | D | T | V | L |
| A-DOPARS-3 | T | L | V | M | D | N | L | V |
| A-DOPARS-4 | Q | M | V | S | D | T | V | I |
| A-DOPARS-5 | E | V | V | Q | G | M | A | V |
| A-DOPARS-6 | E | L | V | Q | G | M | A | T |
| A-DOPARS-7 | T | I | V | M | D | M | A | K |
| A-DOPARS-8 | T | I | V | M | D | M | A | K |
| A-DOPARS-9 | E | V | M | Q | G | M | A | T |
| A-LysN3RS- | V | V | V | A | D | T | S | I |
| 1.1 | | | | | | | | |
| A-LysN3RS- | A | M | V | Q | D | T | V | V |
| 1.2 | | | | | | | | |
| A-LysN3RS- | L | V | V | N | D | I | L | Q |
| 1.3 | | | | | | | | |
| A-LysN3RS- | I | L | V | Q | D | G | L | Q |
| 1.5 | | | | | | | | |
| A-LysN3RS- | I | L | V | Q | D | V | A | L |
| 1.9 | | | | | | | | |
| A-LysN3RS- | A | M | V | Q | D | T | V | G |
| 1.10 | | | | | | | | |
| A-LysN3RS- | L | D | V | L | D | T | L | V |
| 1.11 | | | | | | | | |
| A-BPheRS-1 | E | I | V | L | S | I | S | E |
| A-BPheRS-2 | G | T | V | N | S | V | E | I |
| A-BPheRS-3 | A | L | V | P | D | T | L | A |
| A-BPheRS-4 | G | T | V | N | S | V | E | I |
| A-BPheRS-5 | M | V | R | Q | D | L | A | S |
| A-BPheRS-6 | V | V | L | V | D | T | A | L |
| A-BPheRS-7 | G | T | V | N | S | V | E | I |
| A-BPheRS-8 | L | V | V | N | D | L | S | Q |
| A-BPheRS-9 | G | T | V | N | S | V | E | I |
| A-BPheRS-10 | E | I | V | L | S | I | S | E |
| A-BPheRS-11 | G | T | V | N | S | V | E | I |
| A-BPheRS-12 | G | T | V | N | S | V | E | I |
| A-ATyrRS-4 | L | L | V | M | D | Q | A | S |
| A-ATyrRS-6 | L | V | V | D | D | T | A | E |
| A-ATyrRS-7 | L | M | V | N | D | I | S | L |
| A-ATyrRS-8 | L | L | V | M | D | Q | A | S |
| A-ATyrRS-10 | E | V | V | H | D | V | L | I |
| B-OPGRS-H1 | V | V | T | Q | G | M | A | Q |
| B-OPGRS-H2 | I | L | V | Q | G | M | A | Q |
| B-OPGRS-L1 | I | L | V | Q | G | M | A | Q |
| B-OPGRS-L2 | I | L | V | Q | G | M | A | Q |
| B-OPGRS-L3 | I | L | V | Q | G | M | A | Q |
| B-OPGRS-L4 | I | L | V | Q | G | M | A | Q |
| B-OPGRS-L5 | I | L | V | Q | G | M | A | Q |
| B-OPGRS-L6 | V | I | V | Q | G | M | A | Q |
| B-OPGRS-L7 | I | L | V | Q | G | M | A | Q |
| B-OPGRS-L8 | T | L | V | Q | G | M | A | Q |
| B-OPGRS-L9 | I | L | V | Q | G | M | A | Q |
| B-OPGRS-L10 | M | L | V | Q | S | R | L | Q |
| SpecOPGRS-1 | A | V | V | P | S | L | A | E |
| SpecOPGRS-2 | T | V | H | Q | G | M | A | Q |
| SpecOPGRS-3 | G | T | V | A | S | L | A | E |
| SpecOPGRS-4 | G | V | Q | Q | S | T | A | E |
| SpecOPGRS-5 | G | T | V | A | S | L | A | E |
| SpecOPGRS-7 | T | L | V | E | S | T | L | L |
| SpecOPGRS-9 | T | V | A | Q | G | M | A | Q |
| SpecOPGRS-10 | G | T | V | A | S | L | A | E |
| SpecOPGRS-12 | T | V | T | Q | G | M | A | Q |
| PolyT1RS-2 | L | L | V | Q | G | M | A | Q |
| PolyT1RS-3 | I | L | V | Q | G | M | A | Q |
| PolyT1RS-4 | I | L | V | Q | G | M | A | Q |
| PolyT1RS-5 | I | L | V | Q | G | M | A | Q |
| PolyT1RS-6 | I | L | V | Q | G | M | A | Q |
| PolyT1RS-7 | V | L | V | Q | G | M | A | Q |
| PolyT1RS-9 | I | L | V | Q | G | M | A | Q |
| PolyT1RS-10 | I | L | V | Q | G | M | A | Q |
| PolyT1RS-11 | I | L | V | Q | G | M | A | Q |
| PolyT1RS-12 | T | L | V | Q | G | M | A | Q |
| Poly T2RS-2 | L | V | V | Q | G | M | A | Q |
| Poly T2RS-3 | I | L | V | Q | G | M | A | Q |
| PolyT2RS-4 | V | V | V | Q | G | M | A | Q |
| PolyT2RS-5 | V | V | I | Q | G | M | A | Q |
| Poly T2RS-6 | I | V | V | Q | G | M | A | Q |
| PolyT2RS-8 | I | L | V | Q | G | M | A | Q |
| PolyT2RS-9 | V | V | I | Q | G | M | A | Q |
| PolyT2RS-10 | V | V | V | Q | G | M | A | Q |
| PolyT2RS-11 | L | V | V | Q | G | M | A | Q |

TABLE 9
LeuRS clone mutations.

| | LeuRS Position | | | | | | | |
|---|---|---|---|---|---|---|---|---|
| | M40 | L41 | T252 | S496 | Y499 | Y527 | H537 | |
| | Codon | | | | | | | |
| | VNK | VNK | GCT | RST | NNY | NNT | NNT | Notes |
| A-LysN3RS-1 | G | P | T | T | C | Y | G | |
| A-LysN3RS-2 | L | P | T | T | I | G | G | |
| A-LysN3RS-3 | A | G | T | S | G | N | C | |
| A-LysN3RS-1.4 | G | P | T | T | L | G | F | |
| A-LysN3RS-1.6 | A | N | T | S | N | G | F | |
| A-LysN3RS-1.7 | G | T | T | G | T | T | G | |
| A-LysN3RS-1.8 | G | E | T | T | C | Y | G | |
| A-ATyrRS-1 | L | T | T | A | H | S | G | |
| A-ATyrRS-2 | L | T | T | A | H | S | G | |
| A-ATyrRS-3 | L | T | T | A | H | S | G | |
| A-ATyrRS-5 | L | T | T | A | H | S | G | |
| A-ATyrRS-9 | L | T | T | A | G | T | G | |
| A-ATyrRS-12 | Q | G | T | S | Y | Y | H | |
| SpecOPGRS-6 | L | T | A | A | T | T | G | |
| SpecOPGRS-8 | L | T | A | A | C | C | G | |
| SpecOPGRS-11 | L | T | A | A | T | T | G | |
| PolyT1RS-8 | L | A | A | A | S | D | S | |
| Poly T2RS-7 | G | E | A | T | H | F | G | Q2K mutation |
| PolyT2RS-12 | G | A | A | A | S | F | A | |
| B-BockRS-1 | P | A | A | G | G | I | G | |
| B-BockRS-2 | A | G | A | S | G | T | G | |
| B-BockRS-3 | G | A | A | A | C | I | G | |
| B-BockRS-4 | A | G | A | S | G | T | G | |
| B-BockRS-5 | A | G | A | S | G | T | G | |
| B-BockRS-6 | A | G | A | S | G | T | G | |
| B-BockRS-7 | A | G | A | S | G | T | G | |
| B-BockRS-8 | A | G | A | S | G | T | G | |
| B-BockRS-9 | G | G | A | T | T | F | G | |
| B-BockRS-10 | P | G | A | G | A | V | G | |
| B-BockRS-11 | P | G | A | S | L | T | G | |
| B-BockRS-12 | A | A | A | G | A | C | G | |
| B-LysAlkRS-1 | A | G | A | S | V | I | G | |
| B-LysAlkRS-2 | S | P | A | G | T | V | G | |
| B-LysAlkRS-3 | M | H | A | G | A | G | G | |
| B-LysAlkRS-4 | G | V | A | G | T | V | L | |
| B-LysAlkRS-5 | P | G | A | G | C | C | G | |
| B-LysAlkRS-6 | A | G | A | S | G | T | G | |
| B-LysAlkRS-7 | A | G | A | S | G | T | G | |
| B-LysAlkRS-8 | A | G | A | S | G | T | G | |
| B-LysAlkRS-9 | A | G | A | S | G | T | G | |
| B-LysAlkRS-10 | M | L | A | G | F | S | G | |
| B-LysAlkRS-11 | A | G | A | S | G | T | G | |
| B-LysAlkRS-12 | A | G | A | S | G | T | G | |
| B-LysN3RS-1 | G | P | A | G | A | C | G | |
| B-LysN3RS-2 | P | G | A | G | A | V | G | |
| B-LysN3RS-3 | P | G | A | G | A | C | G | |
| B-LysN3RS-4 | P | G | A | G | C | C | G | |
| B-LysN3RS-5 | P | G | A | G | C | C | G | |
| B-LysN3RS-6 | A | P | A | G | G | H | G | |
| B-LysN3RS-7 | S | P | A | G | I | H | G | |
| B-LysN3RS-8 | A | P | A | G | G | H | G | |
| B-LysN3RS-9 | A | G | A | S | G | T | G | |
| B-LysN3RS-10 | A | G | A | S | G | T | G | |
| B-LysN3RS-11 | S | P | A | G | I | H | G | |
| B-LysN3RS-12 | P | G | A | G | C | C | G | |

### Example 4. Error-Prone Polymerase Chain Reaction (epPCR) Library Construction and Screening

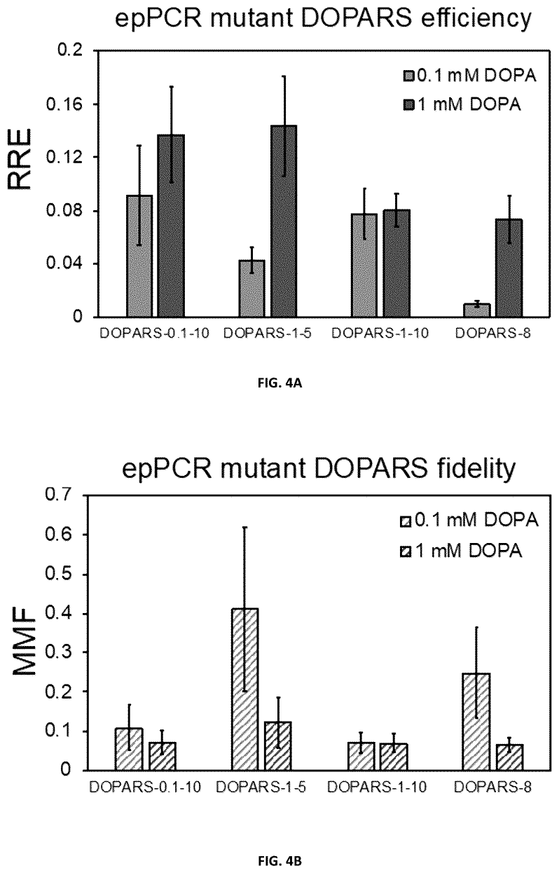

While aminoacyl-tRNA synthetases (aaRSs) capable of charging new noncanonical amino acids (ncAAs) were obtained from the EcTyrRS (E. coli Tyrosyl-tRNA synthetase) and E. coli Leucyl-tRNA synthetase (EcLeuRS) libraries from the initial sorts, some clones exhibited low support of stop codon readthrough. Work was done to improve one of these aminoacyl-tRNA synthetases (aaRSs) for charging of its cognate noncanonical amino acid (ncAA). EcTyrRS (E. coli Tyrosyl-tRNA synthetase) mutants that charge DOPA (7), A-DOPARS-8, was amplified via PCR with two concentrations of mutagenic dNTPs to cause random point mutations across the aminoacyl-tRNA synthetase (aaRS) gene. The lower concentration (1×) of mutagenic reagents was expected to cause 1-3 point mutations and the higher concentration (5×) was expected to cause 5-15 point mutations per gene. The two error-prone polymerase chain reaction (epPCR) libraries, DOPARS-1× and DOPARS-5×, respectively, were constructed in S. cerevisiae RJY100 with 1.6×10⁷ transformants for DOPARS-1× and 2×10⁷ transformants for DOPARS-5×. Based on sequence characterization of 12 clones per library, the average number of point mutations in each library were four for DOPARS-1× and 17 for DOPARS-5×. Both libraries were screened via fluorescence-activated cell sorting (FACS) at both 1 mM and 0.1 mM DOPA. After one negative and four positive screens, with gradually reduced DOPA concentrations, aminoacyl-tRNA synthetases (aaRSs) with error-prone polymerase chain reaction (epPCR)-manufactured mutations outside of the active site were identified by their improved ability to charge DOPA as compared to the parent aminoacyl-tRNA synthetase (aaRS) at one or both noncanonical amino acid (ncAA) concentrations used in screening (FIGS. 4A-4C). The improved incorporation of DOPA with the error-prone polymerase chain reaction (epPCR) mutant aminoacyl-tRNA synthetases (aaRSs) demonstrates how moderately active aminoacyl-tRNA synthetases (aaRSs) evolved for noncanonical amino acid (ncAA) incorporation using the yeast screening platform can be improved using error-prone polymerase chain reaction (epPCR) and further sorting.

Sequence comparison between error-prone polymerase chain reaction (epPCR) DOPARS mutants (Table 10) revealed trends in the locations in the aminoacyl-tRNA synthetases (aaRSs) where mutations were more likely to occur. Many mutations occurred in or directly adjacent to the active site or in the tRNA binding domain.

TABLE 10
Error-prone PCR DOPARS sequences.

| Clone | Q18 | D21 | T37 | L49 | K59 | Q63 | V108 | A109 | F111 | N132 | M179 | C185 |
|---|---|---|---|---|---|---|---|---|---|---|---|---|
| DOPARS-0.1-1 | — | — | — | S | — | — | — | — | — | — | — | — |
| DOPARS-0.1-2 | — | — | G | — | — | — | — | — | L | — | N | — |
| DOPARS-0.1-3 | — | — | — | — | — | — | — | — | — | — | — | — |
| DOPARS-0.1-5 | — | — | — | — | — | — | — | — | — | — | — | — |
| DOPARS-0.1-6 | — | — | — | — | R | — | — | — | — | — | — | — |
| DOPARS-0.1-7 | — | — | — | — | R | — | — | — | — | — | — | — |
| DOPARS-0.1-9 | — | A | — | — | — | — | — | — | — | — | — | Y |
| DOPARS-0.1-10 | — | — | — | — | — | — | — | — | — | — | — | R |
| DOPARS-0.1-11 | — | — | — | — | — | — | — | V | — | — | — | — |
| DOPARS-0.1-12 | — | — | — | — | R | — | — | — | L | — | — | — |
| DOPARS-1-2 | — | — | — | — | R | — | — | — | — | — | — | — |
| DOPARS-1-3 | — | — | — | — | — | — | — | — | L | — | — | — |
| DOPARS-1-4 | — | — | — | — | — | — | — | — | L | — | — | — |
| DOPARS-1-5 | — | — | — | — | — | I | — | — | — | — | — | — |
| DOPARS-1-7 | — | — | — | — | — | — | — | — | — | S | — | — |
| DOPARS-1-8 | — | — | — | — | — | — | — | — | — | — | — | R |
| DOPARS -1-9 | — | — | — | — | — | — | — | — | — | — | — | — |
| DOPARS-1-10 | — | — | — | — | — | — | — | — | — | — | — | — |
| DOPARS-1-11 | — | — | — | — | — | — | — | — | — | — | — | — |
| DOPARS-1-12 | R | — | — | — | — | — | — | — | — | — | — | — |
| Clone | A186 | K195 | I209 | T231 | G242 | T263 | A264 | F277 | M278 | I280 | S293 | Q300 |
| DOPARS-0.1-1 | — | — | — | — | — | — | — | — | — | — | — | — |
| DOPARS-0.1-2 | — | N | — | — | — | — | — | — | — | — | — | R |
| DOPARS-0.1-3 | — | — | — | I | — | — | — | — | — | — | — | — |
| DOPARS-0.1-5 | T | — | — | — | D | — | — | — | — | — | — | — |
| DOPARS-0.1-6 | — | — | — | — | — | — | — | — | — | — | — | — |
| DOPARS-0.1-7 | — | — | — | — | — | — | — | — | — | — | — | — |
| DOPARS-0.1-9 | — | — | — | — | — | — | Q | — | — | T | — | — |
| DOPARS-0.1-10 | — | — | V | — | — | — | — | — | — | — | — | — |
| DOPARS-0.1-11 | — | — | — | — | — | — | — | — | — | — | — | — |
| DOPARS-0.1-12 | — | — | — | — | — | — | — | — | — | — | G | — |
| DOPARS-1-2 | — | — | — | — | — | — | T | — | — | — | — | — |
| DOPARS-1-3 | — | — | — | — | — | — | — | — | V | — | — | — |
| DOPARS-1-4 | — | — | — | — | — | — | — | — | — | — | — | — |
| DOPARS-1-5 | — | — | — | — | — | — | — | L | — | — | — | — |
| DOPARS-1-7 | V | — | — | — | — | — | — | — | — | — | — | — |
| DOPARS-1-8 | — | — | — | — | — | — | — | — | — | — | — | — |
| DOPARS -1-9 | — | — | — | — | — | — | — | — | — | — | — | — |
| DOPARS-1-10 | V | — | V | — | — | A | — | — | — | — | — | — |
| DOPARS-1-11 | T | — | — | — | — | — | — | — | — | — | — | — |
| DOPARS-1- 12 | — | — | — | — | — | A | — | — | — | — | — | — |
| Clone | K321 | M353 | M360 | Q369 | K377 | T378 | N372 | E389 | K390 | S392 | Y396 | K415 |
| DOPARS-0.1-1 | — | — | — | — | — | — | — | — | — | — | — | — |
| DOPARS-0.1-2 | — | — | — | — | — | — | — | — | — | — | — | — |
| DOPARS-0.1-3 | — | — | — | — | — | — | — | — | — | — | — | — |
| DOPARS-0.1-5 | — | — | — | — | — | — | — | — | — | P | — | N |
| DOPARS-0.1-6 | — | — | — | — | — | — | — | — | — | — | — | — |
| DOPARS-0.1-7 | — | — | — | — | — | — | — | — | — | — | — | — |
| DOPARS-0.1-9 | — | — | — | — | E | — | — | D | — | — | — | — |
| DOPARS-0.1-10 | — | V | — | — | — | — | — | — | — | — | H | — |
| DOPARS-0.1- 11 | R | — | — | — | — | — | — | — | — | — | — | — |
| DOPARS-0.1-12 | — | — | — | — | — | — | — | — | — | — | — | — |
| DOPARS-1-2 | — | — | — | — | — | — | — | — | — | — | — | — |
| DOPARS-1-3 | — | V | — | — | — | A | S | — | — | — | — | — |
| DOPARS-1-4 | — | — | — | — | — | — | — | — | — | — | — | — |
| DOPARS-1-5 | — | — | — | — | — | — | — | — | — | — | — | — |
| DOPARS-1-7 | — | — | T | R | — | — | — | — | — | — | — | — |
| DOPARS-1-8 | — | — | — | — | — | — | — | — | — | — | — | — |
| DOPARS -1-9 | — | — | — | — | — | — | — | — | R | — | — | — |
| DOPARS-1-10 | — | — | — | — | — | — | — | — | — | — | — | — |
| DOPARS-1-11 | — | — | — | — | — | — | — | — | — | — | — | — |
| DOPARS-1- 12 | — | — | — | — | — | — | — | — | R | — | — | — |

### Example 5. Modified Screens for Isolation of Aminoacyl-tRNA Synthetases (aaRSs) with Desired Specificity Profiles

With a robust aminoacyl-tRNA synthetase (aaRS) screening platform in place, it was then determined how introducing different induction conditions during fluorescence-activated cell sorting (FACS) could be utilized to isolate highly specific aminoacyl-tRNA synthetases (aaRSs); i.e., aminoacyl-tRNA synthetases (aaRSs) that charge a single noncanonical amino acid (ncAA) to the cognate tRNA_{CUA} (transfer RNA with a CUA anticodon) and do not mischarge other similar noncanonical amino acids (ncAAs). For these experiments, a group of six structurally similar aromatic noncanonical amino acids (ncAAs) was used: OmeY (1), AcF (2), AzF (3), OPG (4), AzMF (5), and IPhe (6). It was expected that the similarity of these noncanonical amino acids (ncAAs) would make it difficult for some aminoacyl-tRNA synthetases (aaRSs) to selectively charge one over the others. First, isolation of aminoacyl-tRNA synthetases (aaRSs) capable of charging OPG (4) and not the other five non-target noncanonical amino acids (ncAAs) was pursued. The negative screens were modified to add 1 mM of all five non-target noncanonical amino acids (ncAAs) during induction prior to negative screens, while the positive screens (the terms “sorts” and “screens” used interchangeably herein) remain unchanged (1 mM OPG was added during induction for positive sorts). Subsequent evaluation of individual aminoacyl-tRNA synthetase (aaRS) clones isolated from these specificity sorts demonstrated that the addition of non-target noncanonical amino acid (ncAA) analogs during negative sort rounds yielded aminoacyl-tRNA synthetases (aaRSs) specific to a single noncanonical amino acid (ncAA) out of a group of 6 noncanonical amino acid (ncAA) analogs.

### Example 6. Modified Screens for Isolation of Aminoacyl-tRNA Synthetases (aaRSs) with Desired Polyspecificity Profiles

In parallel to tuning the specificity of aminoacyl-tRNA synthetases (aaRSs), as described above, during screening, it was also sought to investigate whether the polyspecificity of the aminoacyl-tRNA synthetases (aaRSs) could be enhanced using a different screening strategy. Adaptations to each screen by inducing the library populations with different combinations of noncanonical amino acids (ncAAs) allowed for the determination of whether polyspecific aminoacyl-tRNA synthetases (aaRSs) could be isolated from the Tyrosyl-tRNA synthetase (TyrRS) and Leucyl tRNA synthetase (LeuRS) libraries. For the first track (Track 1, T1), all six of the same group of aromatic noncanonical amino acids (ncAAs) (OmeY (1), AcF (2), AzF (3), OPG (4), AzMF (5), and IPhe (6)) were added in the induced cultures for all positive sort rounds. For the second track (Track 2, T2), only one of the six aromatic noncanonical amino acids (ncAAs) was added per positive sort round, with a different noncanonical amino acid (ncAA) for each subsequent positive sort round. With 3 consecutive positive sorts followed by a single negative sort, characterization of 11 clones from the Track 1 screens yielded four unique Tyrosyl-tRNA synthetase (TyrRS) mutants and one Leucyl tRNA synthetase (LeuRS) mutant. All four of the unique Tyrosyl-tRNA synthetase (TyrRS) variants differed only by the first mutated position (Y37 to L, I, V, or T). For Track 2 sorts, the pooled libraries were screened first using AzF (3), followed by a positive screen for incorporation of AcF (2), a negative screen, and two consecutive positive screens for AzMF (5) due to lower incorporation of AzMF (5) as compared to the other five noncanonical amino acids (ncAAs) during flow cytometry characterization of intermediate populations. Sequence characterization of 12 aminoacyl-tRNA synthetases (aaRSs) from the T2 sorted library population yielded six unique clones: four Tyrosyl-tRNA synthetase (TyrRS) variants and two Leucyl tRNA synthetase (LeuRS) variants. An identical sequence to TyrAcFRS (Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269; Van Deventer, J. A., Le, D. N., Zhao, J., Kehoe, H. P., and Kelly, R. L. (2016) A platform for constructing, evaluating, and screening bioconjugates on the yeast surface, Protein Eng Des Sel 29, 485-494) was found seven times in the Track 1 population out of the 11 clones and two times in the Track 2 population out of the 12 clones evaluated, further indicating its polyspecific ability to charge five of the six noncanonical amino acids (ncAAs) tested. No other sequence consensus occurred from Track 1 sorts, but clone PolyT2RS-5 appeared five times out of 12 clones that were characterized from Track 2.

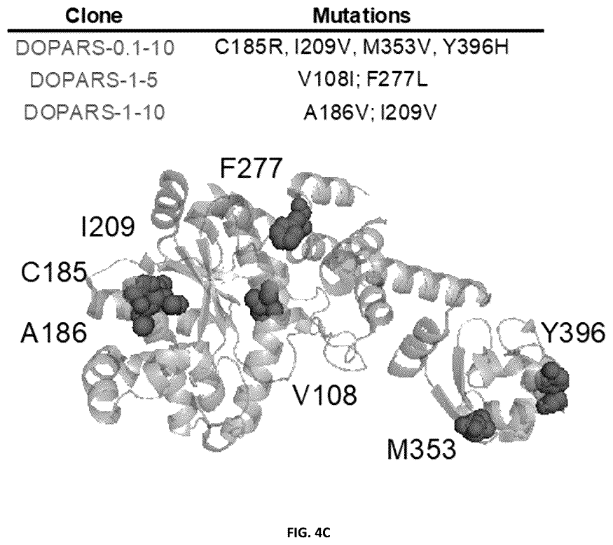

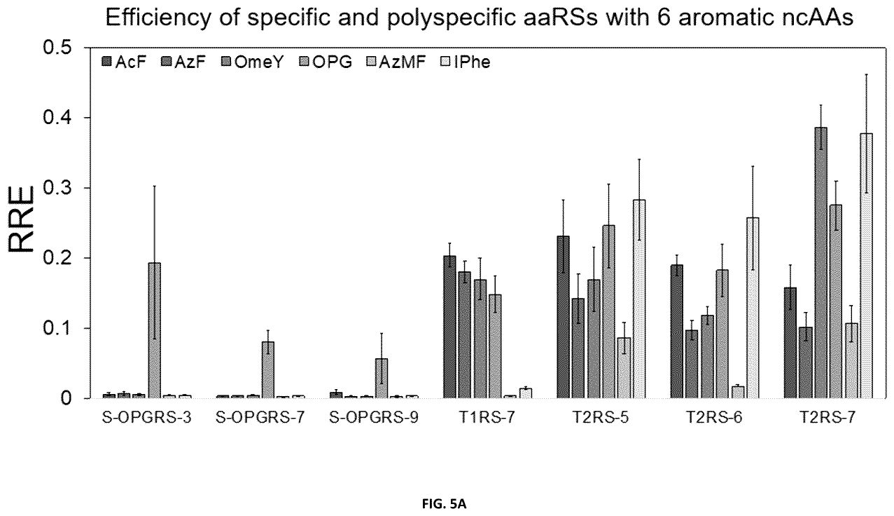

Evaluation of the efficiency and fidelity of the unique polyspecific aminoacyl-tRNA synthetases (aaRSs) demonstrated that the aminoacyl-tRNA synthetases (aaRSs) were able to encode several of the group of six noncanonical amino acids (ncAAs), and also revealed a difference in outcome between the two sort tracks (FIG. 5A and Table 11). In the case of Track 1, the most active clone B-PolyT1RS-7 (T1RS-7 in FIG. 5A) encoded 4 out of the 6 noncanonical amino acids (ncAAs). For Track 2 sorts, when the sorted populations revealed reduced AzMF incorporation during routine flow checks, positive sorts were performed with that noncanonical amino acid (ncAA) to ultimately isolate aminoacyl-tRNA synthetases (aaRSs) that encoded all 6 noncanonical amino acids (ncAAs). By making simple modifications to the induction step prior to fluorescence-activated cell sorting (FACS), aminoacyl-tRNA synthetases (aaRSs) with polyspecific characteristics were isolated for use in applications where a single aminoacyl-tRNA synthetase (aaRS) that can encode multiple noncanonical amino acids (ncAAs) is required.

TABLE 11
Sequences of aminoacyl-tRNA synthetases (aaRSs) from FIG. 5.

| | Position | I37 | L71 | Q179 | G182 | M183 | A186 | Q195 |
|---|---|---|---|---|---|---|---|---|
| TyrRs | B-SOPGRS-3 | G | T | A | S | L | A | E |
| | B-SOPGRS-7 | T | L | E | S | T | L | L |
| | B-SOPGRS-9 | T | V | Q | G | M | A | Q |
| | B-PolyT1RS-7 | V | L | Q | G | M | A | Q |
| | B-PolyT2RS-5 | V | V | Q | G | M | A | Q |
| | B-PolyT2RS-6 | I | V | Q | G | M | A | Q |
| | Position | M40 | L41 | T252 | S496 | Y499 | Y527 | H537 |
| LeurRS | A-LysN3RS-1 | G | P | T | T | C | Y | G |
| | B-PolyT2RS-7 | G | E | A | T | H | F | G |
| | B-LysAlkRS-3 | M | H | A | G | A | G | G |
| | B-LysN3RS-1 | G | P | A | G | A | C | G |
| | B-LysN3RS-7 | S | P | A | G | I | H | G |
| | B-LysN3RS-8 | A | P | A | G | G | H | G |
| | B-BockRS-1 | P | A | A | G | G | I | G |
| | B-BockRS-2 | A | G | A | S | G | T | G |

### Example 7. Characterizing Polyspecific Aminoacyl-tRNA Synthetases (aaRSs) for Activity with Aliphatic Noncanonical Amino Acids (ncAAs)

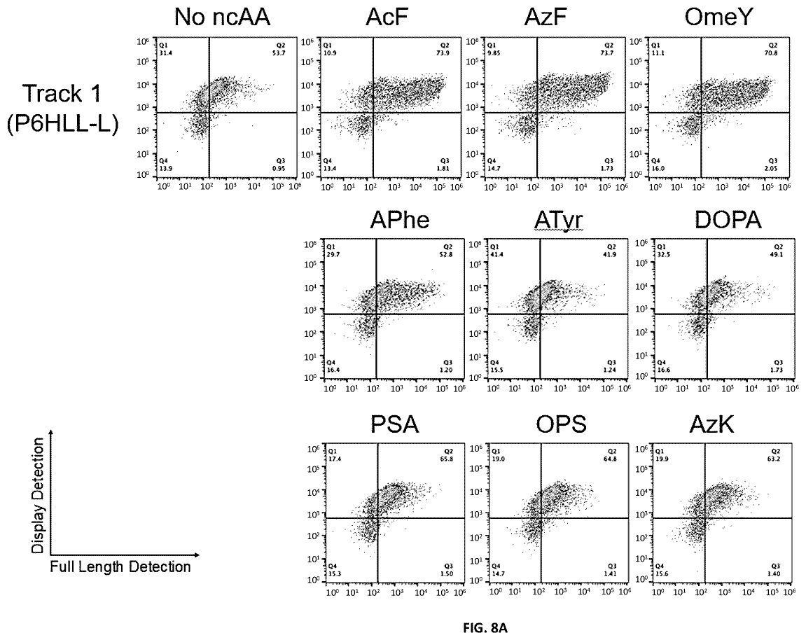

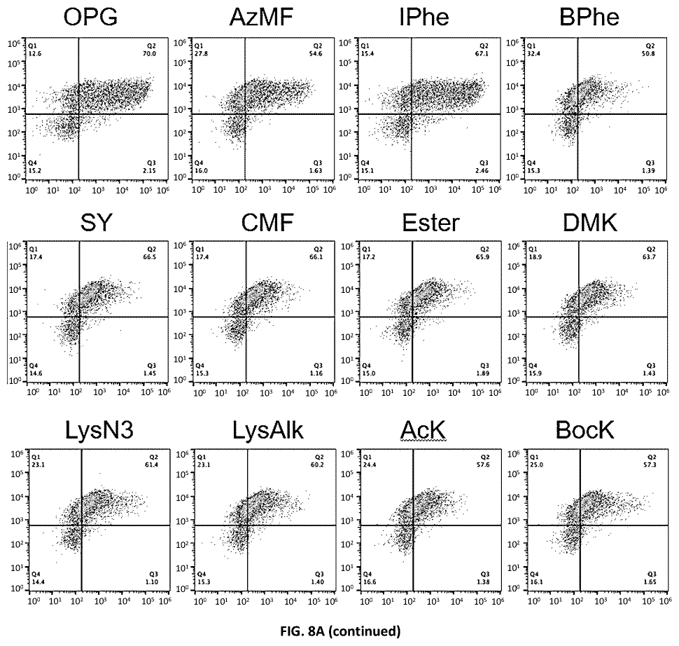

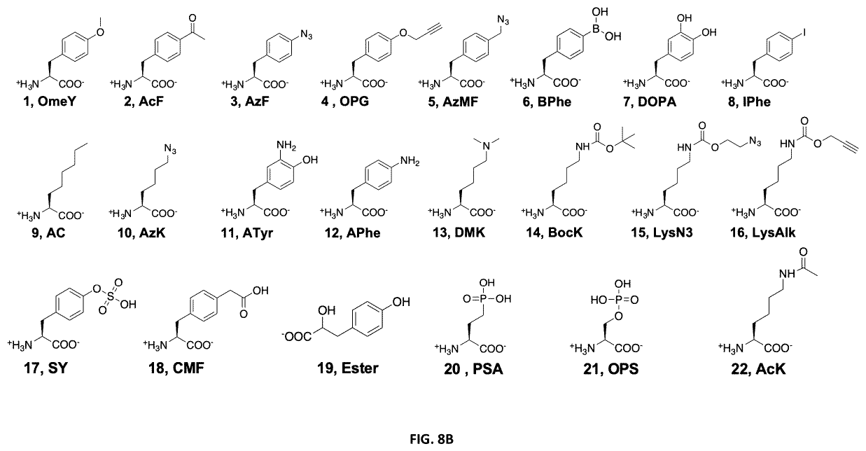

To further evaluate the extent of polyspecificity of the final screened Track 1 library population, the population was tested for incorporation of 21 noncanonical amino acids (ncAAs) (FIGS. 8A-8B). While the Track 1 aminoacyl-tRNA synthetases (aaRSs) were only screened against a group of aromatic noncanonical amino acids (ncAAs), they also encoded several aliphatic noncanonical amino acids (ncAAs) at low levels. To investigate if aminoacyl-tRNA synthetases (aaRSs) from the Track 1 library population were polyspecific for noncanonical amino acids (ncAAs) beyond the original six aromatic noncanonical amino acids (ncAAs), the library was screened for three aliphatic noncanonical amino acids (ncAAs): BocK (14), LysN3 (15) and LysAlk (16). After one positive screen for incorporation of BocK, the population yielded nine Leucyl tRNA synthetase (LeuRS) variants, six of which were unique and one sequence that appeared four times in the population. Similarly, two consecutive positive rounds of screening with LysAlk and LysN3 also yielded only Leucyl tRNA synthetase (LeuRS) mutants. For the LysAlk library population, sequence characterization of 11 clones yielded seven unique sequences and one sequence that appeared five times. The consensus sequence for the LysAlk population was the same clone that appeared multiple times in the BocK population. For the LysN3 population, sequence characterization of 12 clones revealed eight unique Leucyl tRNA synthetase (LeuRS) variants. One of the eight unique Leucyl tRNA synthetase (LeuRS) clones appeared three times, and two other clones appeared twice each, including the same sequence that appeared in both the LysAlk and BocK populations. The efficiency and fidelity of noncanonical amino acid (ncAA) incorporation for several clones from each population was evaluated and some were able to encode the aliphatic noncanonical amino acid (ncAA) target at levels comparable to the Track 1 and Track 2 relative readthrough efficiency (RRE) values for the original six aromatic noncanonical amino acids (ncAAs) with similarly low misincorporation of cAAs (FIG. 5B). While few Leucyl tRNA synthetase (LeuRS) variants resulted from characterization of the original Track 1 and 2 screens, all of the aminoacyl-tRNA synthetases (aaRSs) isolated from the aliphatic noncanonical amino acid (ncAA) sorts were derived from Leucyl tRNA synthetase (LeuRS). Regardless of the parent aminoacyl-tRNA synthetase (aaRS), these results demonstrated that aminoacyl-tRNA synthetases (aaRSs) screened for polyspecific incorporation of aromatic noncanonical amino acids (ncAAs) may also confer polyspecificity for other noncanonical amino acid (ncAA) structures, including the aliphatic BocK, LysAlk, and LysN3 noncanonical amino acids (ncAAs).

### Example 8. Trends in Aminoacyl-tRNA Synthetase (aaRS) Active Site Residues

For both the Tyrosyl-tRNA synthetase (TyrRS) and Leucyl tRNA synthetase (LeuRS) mutants isolated during screening, several trends appeared in which residues in the active site resulted in more efficient aminoacylation of particular groups of noncanonical amino acids (ncAAs). For example, 11 out of the 12 Tyrosyl-tRNA synthetase (TyrRS) variants isolated from non-specificity sorts for OPG contained mutations D182G, F183M, and L186A, and maintained WT residues at positions Q179 and Q195—herein referred to as the QGMAQ motif This was interesting when compared to the OPGRSs isolated from specificity screens, for which the active site residues varied significantly more, though the L186A mutations still appeared for 8 out of 9 aminoacyl-tRNA synthetases (aaRSs). QGMAQ motif may have been removed from the library population during specificity screens because the motif may improve the efficiency of charging OPG to tRNA_{CUA} (transfer RNA with a CUA anticodon) but also allow OPG analogs to be aminoacylated. The QGMAQ motif appears in all of the unique clones from both Track 1 and Track 2 polyspecificity sorts. The only positions that differed between the polyspecific aminoacyl-tRNA synthetases (aaRSs) were positions 37, 71, and 72. Position V72 was not included in the original set of active site residues chosen for mutation and did not appear in the initial Tyrosyl-tRNA synthetase (TyrRS) library characterization. However, position V72 showed up in several of the Tyrosyl-tRNA synthetase (TyrRS) clones isolated from different sort tracks and can be attributed to an error in primer binding during the PCR step of library construction. For the polyspecific aminoacyl-tRNA synthetases (aaRSs), the 37, 71, and 72 positions only contained mutations to leucine, isoleucine, and valine as well as one instance of a Y37T mutation. Not wishing to be bound by theory, these trends in active site residues support the theory that the QGMAQ motif contributes to polyspecific aminoacylation of some noncanonical amino acids (ncAAs), and that small, hydrophobic residues may further alter the active site conformation in a way that reduces selectivity of one noncanonical amino acid (ncAA) over another.

Tyrosyl-tRNA synthetase (TyrRS) variants supporting incorporation of LysN3 showed a similar trend at positions 37 and 71, where a majority of residues were alanine, valine, leucine, and methionine (all hydrophobic). However, the QGMAQ motif did not appear and all seven unique clones retained the wild type (WT) aspartic acid at position 182. LysN3RSs isolated from Leucyl tRNA synthetase (LeuRS) Library A revealed two notable trends: 1) all of the Leucyl tRNA synthetase (LeuRS) clones did not contain the T252A editing domain mutation and 2) a disproportionately high rate of glycine and threonine. The other group of LysN3RSs were variants of Leucyl tRNA synthetase (LeuRS) Library B and were originally screened as polyspecificity Track 1. All 12 of the aminoacyl-tRNA synthetases (aaRSs) isolated from this population were Leucyl tRNA synthetase (LeuRS) variants and contained the T252A mutation. For the six active site positions diversified in the original library construction, some trends were discovered. Positions M40, L41, S496, Y499, and H537 were primarily mutated to glycine, proline, or alanine with a few instances of serine and isoleucine residues. In particular, virtually all of the clones were mutated to glycine at both positions S496 and H537. S496 had not previously been included in a Leucyl tRNA synthetase (LeuRS) saturation mutagenesis library Not wishing to be bound by theory, position Y527 showed the most variability with residues such as cysteine, histidine, and threonine appearing most often. The frequency of glycine and alanine mutations, particularly in clones B-LysN3RS-1, 7, and 8 (all of which demonstrated high levels of efficiency for charging LysN3) supports the possibility that these mutations alter the active site specificity to aminoacylate aliphatic noncanonical amino acids (ncAAs). This theory is further reinforced by comparison of aminoacyl-tRNA synthetase (aaRS) sequences capable of charging aliphatic noncanonical amino acids (ncAAs) LysAlk and BocK.

With one exception, all 24 clones isolated from sorts for BocK and LysAlk encoded glycine at the 537 position in the active site. All clones had the expected T252A mutation, and similarly to the LysN3 mutants, primarily glycine, proline, or alanine at positions M40, L41, and Y499. Residue 496 was virtually always a glycine or serine and residue 527 mainly converged to threonine. In the best performing aminoacyl-tRNA synthetase (aaRS), B-LysAlkRS-3, residue M40 was unmutated and residue L41 was mutated to histidine; the only Leucyl tRNA synthetase (LeuRS) variant isolated from any screen in this work that contained an L41H mutation.

Additionally, trends were investigated between the noncanonical amino acid (ncAA) screening targets and whether more Tyrosyl-tRNA synthetase (TyrRS) or Leucyl tRNA synthetase (LeuRS) variants were isolated from the final library populations after screening. For sorts where a single noncanonical amino acid (ncAA) of interest was the target, aminoacyl-tRNA synthetases (aaRSs) isolated tended to predominantly originate in the Tyrosyl-tRNA synthetase (TyrRS) if the noncanonical amino acid (ncAA) contained a benzyl ring, or Leucyl tRNA synthetase (LeuRS) if the noncanonical amino acid (ncAA) was aliphatic (Tables 8 and 9). Populations of aminoacyl-tRNA synthetases (aaRSs) sorted for the ability to charge OmeY (1), DOPA (7), BPhe (8), OPG (4), and the polyspecificity sorts for groups of aromatic noncanonical amino acids (ncAAs) all had 80-100% Tyrosyl-tRNA synthetase (TyrRS) clones. Similarly, for sorts for charging of aliphatic noncanonical amino acids (ncAAs), aminoacyl-tRNA synthetases (aaRSs) for all three sorts (BocK, LysAlk, and LysN3) that started as polyspecificity sorts against aromatic noncanonical amino acids (ncAAs) but were continued for aliphatic noncanonical amino acids (ncAAs), 100% of clones were Leucyl tRNA synthetase (LeuRS) variants. There were two notable exceptions: LysN3 from sorts with Leucyl tRNA synthetase (LeuRS) Library A and ATyr. For both of these tracks, Tyrosyl-tRNA synthetase (TyrRS) and Leucyl tRNA synthetase (LeuRS) clones appeared an approximately equal number of times from sequence characterization of final populations. For LysN3 this may have been due to the misconstruction of Leucyl tRNA synthetase (LeuRS) Library A, where the last two positions in the active site chosen for mutations were discovered to be mostly wild type (WT) larger residues tyrosine and histidine. For Leucyl tRNA synthetase (LeuRS) variants isolated from Library A screens, none of the aminoacyl-tRNA synthetases (aaRSs) had the wild type (WT) residue histidine at the position 537, which may indicate the importance of mutating that residue for improved interaction of LysN3 with the active site. For ATyr, half of the isolated aminoacyl-tRNA synthetases (aaRSs) were Leucyl tRNA synthetase (LeuRS) variants. Despite the aromatic structure of ATyr compared to tyrosine and leucine, the best aminoacyl-tRNA synthetase (aaRS) for charging ATyr to tRNA_{CUA} (transfer RNA with a CUA anticodon) was a Leucyl tRNA synthetase (LeuRS) variant.

TABLE 12
E. coli TyRS library characterization.

| | WT Codon | | | | | | |
|---|---|---|---|---|---|---|---|
| | Y37 | L71 | Q179 | D182 | F183 | L186 | Q195 |
| | Degenerate codon | | | | | | |
| | VNK + | | | | | | |
| | TAT | VNK | GCT | RRT | VNK | KYA | VNK |
| 1 | G | G | R | N | K | L | V |
| 2 | R | S | R | D | G | A | R |
| 3 | S | V | A | D | A | A | P |
| 4 | L | L | S | D | P | A | Q |
| 5 | D | M | E | D | R | A | L |
| 6 | M | V | L | N | I | L | V |
| 7 | H | D | T | G | M | A | P |
| 8 | S | M | R | S | H | L | P |
| 9 | G | V | N | S | D | A | V |
| 10 | N | V | R | D | N | A | I |

TABLE 13
E. coli LeuRS library characterization.

| | WT Codon | | | | | | |
|---|---|---|---|---|---|---|---|
| | M40 | L41 | T252 | S496 | Y499 | Y527 | H537 |
| | Degenerate codon | | | | | | |
| | VNK | VNK | GCT | RST | NNY | NNT | NNT |
| 1 | N | T | A | T | T | Y | H |
| 2 | A | S | A | T | A | Y | H |
| 3 | V | E | A | T | A | Y | H |
| 4 | A | R | A | G | D | Y | F |
| 5 | G | A | A | T | P | S | A |
| 6 | N | R | A | A | A | Y | T |
| 7 | I | R | A | A | I | Y | L |
| 8 | G | H | A | A | H | V | S |
| 9 | N | R | A | T | S | V | Y |
| 10 | R | D | A | G | Y | Y | H |

TABLE 14
E. coli LeuRS library characterization from
second library construction to correct prodominantily
wildtype residues at the Y527 and H537 positions.

| | WT Codon | | | | | | |
|---|---|---|---|---|---|---|---|
| | M40 | L41 | T252 | S496 | Y499 | Y527 | H537 |
| | Degenerate codon | | | | | | |
| | VNK | VNK | GCT | RST | NNY | NNT | NNT |
| 1 | I | D | A | A | H | Y | S |
| 2 | G | S | A | G | I | H | F |
| 3 | G | H | A | S | D | S | C |
| 4 | A | T | A | T | Y | D | I |
| 5 | A | T | A | T | Y | D | I |
| 6 | L | N | A | A | P | T | N |
| 7 | R | V | A | S | A | V | S |
| 8 | D | H | A | A | S | A | H |
| 9 | M | L | A | G | N | S | S |

### Example 9. Comparison Between Aminoacyl-tRNA Synthetases (aaRSs) Sorted Using Different Methods

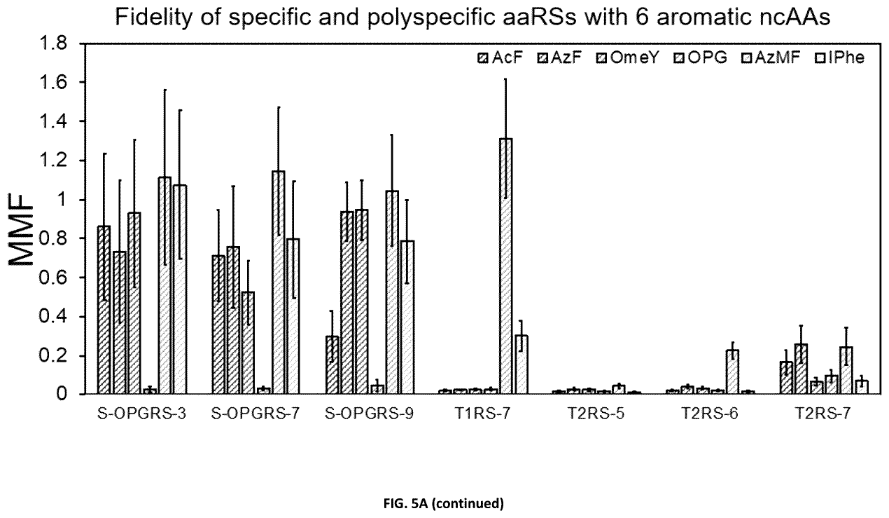

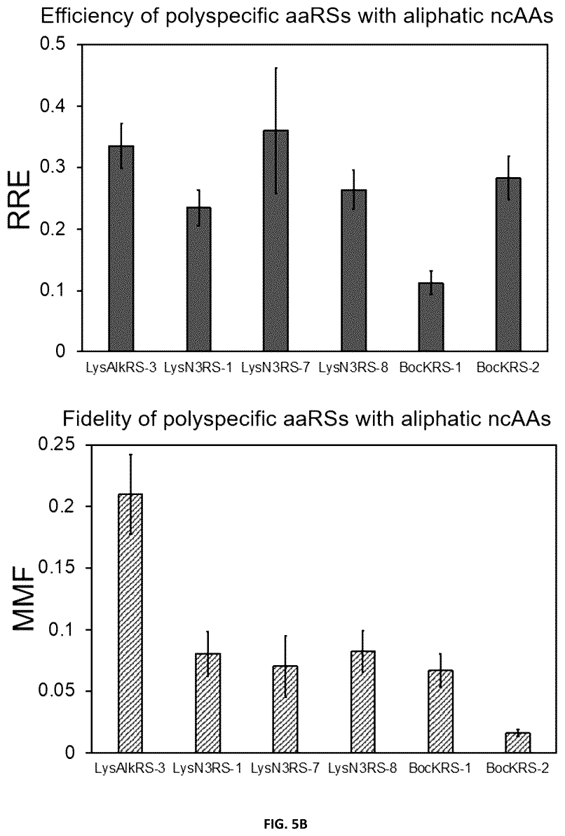

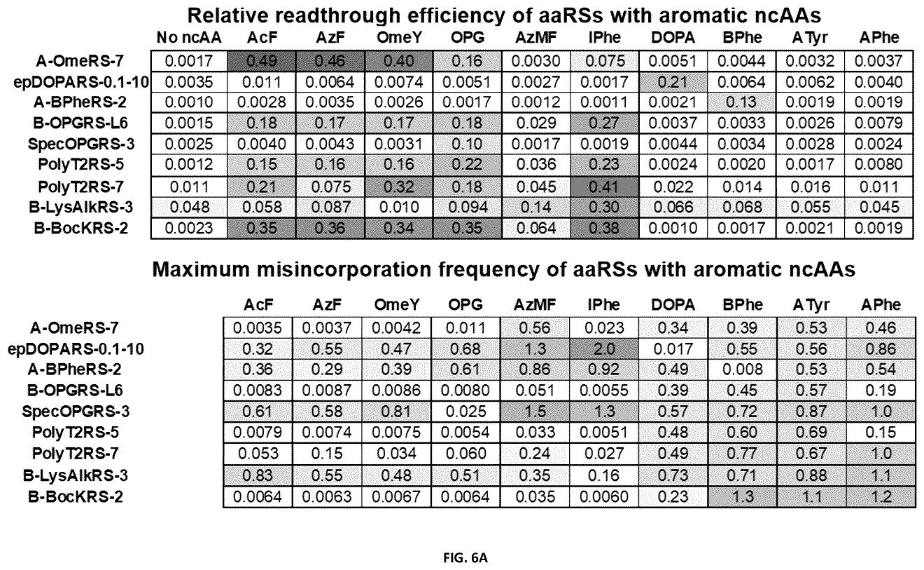

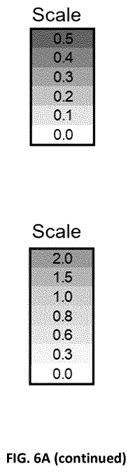

In order to cross-evaluate some of the unique aminoacyl-tRNA synthetases (aaRSs) isolated from the screens with a larger set of noncanonical amino acids (ncAAs), the efficiency and fidelity of nine aminoacyl-tRNA synthetases (aaRSs) was measured with ten aromatic noncanonical amino acids (ncAAs) and eight aminoacyl-tRNA synthetases (aaRSs) with six aliphatic noncanonical amino acids (ncAAs) (FIGS. 6A and 6B, Tables 15-18). For aminoacyl-tRNA synthetases (aaRSs) evolved using a more standard screening strategy rather than one intended to isolate specific or polyspecific aminoacyl-tRNA synthetases (aaRSs), the aminoacyl-tRNA synthetase (aaRS) clones had variable levels of specificity/polyspecificity. For example, the clones A-BPheRS-2 and epDOPARS-0.1-10 both exhibited specificity towards the noncanonical amino acids (ncAAs) BPhe (8) and DOPA (7), respectively, whereas A-OmeRS-7 and B-OPGRS-L6 were able to encode five of the group of 10 aromatic noncanonical amino acids (ncAAs) at moderate to high levels (FIG. 6A, Tables 15 and 16). These observations are consistent with little to no control over incorporation of noncanonical amino acids (ncAAs) not originally included in the screening process. Conversely, for aminoacyl-tRNA synthetases (aaRSs) isolated from intentionally polyspecific or specific sorts, the behavior of the aminoacyl-tRNA synthetases (aaRSs) were consistent with the type of screen employed. Specificity clone SpecOPGRS-3 only supported translation with OPG (4) and not any of the other aromatic noncanonical amino acids (ncAAs) evaluated. Polyspecificity clones PolyT2RS-5 and PolyT2RS-7 performed well with five out of the original six aromatic noncanonical amino acids (ncAAs) they were selected for incorporation of and encoded the sixth noncanonical amino acid (ncAA), AzMF (5), at detectable levels. Interestingly, these clones did not support incorporation of the other aromatic noncanonical amino acids (ncAAs) tested. The two aminoacyl-tRNA synthetases (aaRSs) originally screened with Track 1 polyspecific methods and then carried forward for subsequent screens with aliphatic noncanonical amino acids (ncAAs) LysAlk (16) and BocK (14) both exhibited highly polyspecific behavior toward the original set of six aromatic noncanonical amino acids (ncAAs), notably with lower incorporation of AzMF (5) than the other five. Despite relative readthrough efficiency (RRE) values of near zero, APhe (12) in qualitative plots from flow cytometry appeared to showed low levels of activity, indicating that APhe (12) was able to be encoded by one of the aminoacyl-tRNA synthetase (aaRS) tested (FIG. 9). However, due to the method by which relative readthrough efficiency (RRE) was calculated, cells that lost the suppression machinery and were incapable of reading through the TAG codon were included in the value determination of N- and C-terminus detection, which lowered the overall median fluorescence intensity (MFI). This led to an unanticipatedly low relative readthrough efficiency (RRE) value that does not appear to correspond well to the qualitative flow cytometry plots and was more noticeable for aminoacyl-tRNA synthetases (aaRSs) with lower activity, such as A-ATyrRS-1 and several of the aminoacyl-tRNA synthetases (aaRSs) evaluated in FIG. 6 (see Tables 15-18 for error values).

TABLE 15
Calculated error of RRE measurements of aaRSs with aromatic ncAAs.

| | No | | | | | | | | | | |
|---|---|---|---|---|---|---|---|---|---|---|---|
| | ncAA | AcF | AzF | OmeY | OPG | AzMF | IPhe | DOPA | BPhe | ATyr | APhe |
| A-OmeRS-7 | 0.00014 | 0.080 | 0.27 | 0.12 | 0.056 | 0.00044 | 0.026 | 0.0012 | 0.0057 | 0.00099 | 0.00113 |
| epDOPAR | 0.00058 | 0.0024 | 0.0016 | 0.0017 | 0.0013 | 0.00041 | 0.00034 | 0.050 | 0.0023 | 0.00103 | 0.00097 |
| S-0.1-10 | | | | | | | | | | | |
| A-BPheR | 0.00034 | 0.00064 | 0.00056 | 0.00068 | 0.00057 | 0.00029 | 0.00016 | 0.00076 | 0.048 | 0.00045 | 0.00072 |
| S-2 | | | | | | | | | | | |
| B-OPGR | 0.00032 | 0.039 | 0.040 | 0.079 | 0.052 | 0.016 | 0.096 | 0.0018 | 0.00223 | 0.00184 | 0.00435 |
| S-L6 | | | | | | | | | | | |
| SpecOPGR | 0.0010 | 0.0015 | 0.0017 | 0.0012 | 0.052 | 0.0060 | 0.0059 | 0.0015 | 0.00146 | 0.00093 | 0.00085 |
| S-3 | | | | | | | | | | | |
| PolyT2R S-5 | 0.00015 | 0.048 | 0.063 | 0.052 | 0.054 | 0.011 | 0.094 | 0.00043 | 0.00034 | 0.0003 | 0.00321 |
| PolyT2R S-7 | 0.0026 | 0.028 | 0.017 | 0.18 | 0.041 | 0.0080 | 0.051 | 0.0041 | 0.00364 | 0.00459 | 0.00317 |
| B-LysAlKR | 0.0041 | 0.0080 | 0.013 | 0.011 | 0.011 | 0.016 | 0.068 | 0.0072 | 0.01127 | 0.00736 | 0.00407 |
| S-3 | | | | | | | | | | | |
| B-BocKR | 0.00041 | 0.054 | 0.090 | 0.13 | 0.062 | 0.024 | 0.15 | 0.0036 | 0.00036 | 0.00041 | 0.00043 |
| S-2 | | | | | | | | | | | |

TABLE 16
Calculated error of MMF measurements of aaRSs with aromatic ncAAs.

| | AcF | AzF | OmeY | OPG | AzMF | IPhe | DOPA | BPhe | ATyr | APhe |
|---|---|---|---|---|---|---|---|---|---|---|
| OmeR S-7 | 0.00064 | 0.0022 | 0.0013 | 0.0040 | 0.095 | 0.0081 | 0.087 | 0.060 | 0.17 | 0.14 |
| epDOPAR S-0.1- | 0.089 | 0.16 | 0.14 | 0.21 | 0.30 | 0.51 | 0.0049 | 0.22 | 0.13 | 0.25 |
| 10 | | | | | | | | | | |
| BPheR S-2 | 0.15 | 0.11 | 0.16 | 0.29 | 0.35 | 0.33 | 0.24 | 0.0040 | 0.21 | 0.27 |
| OPGR S-L6 | 0.0026 | 0.0027 | 0.0043 | 0.0029 | 0.030 | 0.0023 | 0.21 | 0.32 | 0.43 | 0.11 |
| SOPGR S-3 | 0.34 | 0.33 | 0.46 | 0.016 | 0.79 | 0.65 | 0.30 | 0.42 | 0.45 | 0.55 |
| T2R S-5 | 0.0027 | 0.0031 | 0.0027 | 0.0015 | 0.011 | 0.0022 | 0.10 | 0.13 | 0.15 | 0.062 |
| T2R S-7 | 0.014 | 0.048 | 0.021 | 0.020 | 0.071 | 0.0071 | 0.15 | 0.27 | 0.24 | 0.36 |
| LysAlkR S-3 | 0.15 | 0.096 | 0.067 | 0.075 | 0.050 | 0.038 | 0.10 | 0.13 | 0.14 | 0.13 |
| BocKR S-2 | 0.0015 | 0.0020 | 0.0028 | 0.0016 | 0.015 | 0.0026 | 0.092 | 0.36 | 0.29 | 0.35 |

TABLE 17
Calculated error of RRE measurements of aaRSs with aliphatic ncAAs.

| | No ncAA | LysN3 | LysAlk | BocK | AzK | DMK | AC |
|---|---|---|---|---|---|---|---|
| Poly T2R | 0.00026 | 0.00027 | 0.00021 | 0.00030 | 0.00035 | 0.00029 | 0.00027 |
| S-5 | | | | | | | |
| PolyT2R | 0.0032 | 0.0075 | 0.0021 | 0.0024 | 0.0049 | 0.0042 | 0.025 |
| S-7 | | | | | | | |
| B-LysAlkR | 0.0041 | 0.020 | 0.024 | 0.011 | 0.0021 | 0.0035 | 0.0083 |
| S-3 | | | | | | | |
| B-BocKR | 0.00042 | 0.0057 | 0.0011 | 0.033 | 0.00038 | 0.0013 | 0.0043 |
| S-2 | | | | | | | |
| B-LysN3R | 0.0035 | 0.051 | 0.0022 | 0.0020 | 0.0013 | 0.0021 | 0.0033 |
| S-7 | | | | | | | |
| LeuOmeR | 0.00027 | 0.00022 | 0.00018 | 0.00016 | 0.00026 | 0.00023 | 0.00036 |
| S | | | | | | | |
| A-OmeR | 0.00016 | 0.00034 | 0.00034 | 0.0003 | 0.00031 | 0.00031 | 0.00025 |
| S-7 | | | | | | | |
| B-OPGR | 0.00018 | 0.00060 | 0.00046 | 0.00047 | 0.00014 | 0.00039 | 0.00022 |
| S-L6 | | | | | | | |

TABLE 18
Calculated error of MMF measuremetns
of aaRSs with aliphatic ncAAs.

| | LysN3 | LysAlk | BocK | AzK | DMK | AC |
|---|---|---|---|---|---|---|
| Poly T2R | 0.24 | 0.29 | 0.51 | 0.23 | 0.27 | 0.37 |
| S-5 | | | | | | |
| PolyT2R | 0.12 | 0.35 | 0.49 | 0.24 | 0.36 | 0.039 |
| S-7 | | | | | | |
| B-LysAlkR | 0.011 | 0.018 | 0.068 | 0.11 | 0.10 | 0.053 |
| S-3 | | | | | | |
| B-BocKR | 0.0091 | 0.018 | 0.0030 | 0.078 | 0.046 | 0.012 |
| S-2 | | | | | | |
| B-LysN3R | 0.013 | 0.35 | 0.35 | 0.31 | 0.33 | 0.28 |
| S-7 | | | | | | |
| LeuOmeR | 0.16 | 0.34 | 0.46 | 0.23 | 0.28 | 0.22 |
| S | | | | | | |
| A-OmeR | 0.074 | 0.086 | 0.10 | 0.12 | 0.16 | 0.13 |
| S-7 | | | | | | |
| B-OPGR | 0.092 | 0.087 | 0.13 | 0.11 | 0.17 | 0.15 |
| S-L6 | | | | | | |

Several aminoacyl-tRNA synthetases (aaRSs) were also evaluated against a panel of aliphatic noncanonical amino acids (ncAAs) (FIG. 6B, Tables 17 and 18). Polyspecificity clone PolyT2RS-5 showed virtually no incorporation of any of the aliphatic noncanonical amino acids (ncAAs) tested but PolyT2RS-7 incorporated AC (9) with a relative readthrough efficiency (RRE) value of 0.12. LeuOmeRS, A-OmeRS-7, and B-OPGRS-L6, all of which were not sorted for polyspecific characteristics but demonstrated them with aromatic noncanonical amino acids (ncAAs), did not encode any of the aliphatic noncanonical amino acids (ncAAs) tested. The highest performing aliphatic noncanonical amino acid (ncAA)-encoding aminoacyl-tRNA synthetases (aaRSs) that were screened for aromatic noncanonical amino acid (ncAA) polyspecificity before being screened for incorporation of individual aliphatic noncanonical amino acids (ncAAs) were B-LysAlkRS-3, B-BocKRS-2, and B-LysN3RS-7. B-LysN3RS-7 encoded LysN3 (15) efficiently with a relative readthrough efficiency (RRE) value of 0.31 but did not encode any other noncanonical amino acids (ncAAs) tested. B-BocKRS-2 similarly encoded BocK (14) well but showed little to no activity for any other aliphatic noncanonical amino acids (ncAAs). Unlike its BocK (14) and LysN3 (15) counterparts, B-LysAlkRS-3 not only encoded LysAlk (16) efficiently with a relative readthrough efficiency (RRE) of 0.30, but also encoded LysN3 (15), BocK (14), and AC (9). B-LysAlkRS-3 had a slightly higher background level of canonical amino acid (cAA) misincorporation, which manifested in the maximum misincorporation frequency (MMF) values. For B-LysAlkRS-3 and B-BocKRS-2, a few additional screens for aliphatic noncanonical amino acids (ncAAs) yielded aminoacyl-tRNA synthetases (aaRSs) that were polyspecific for both the original six aromatic noncanonical amino acids (ncAAs) used in the polyspecificity screens as well as aliphatic noncanonical amino acids (ncAAs).

A difference in polyspecificity between Tyrosyl-tRNA synthetase (TyrRS) and Leucyl tRNA synthetase (LeuRS) variants resulting from the screening methods and targets was observed. Select Tyrosyl-tRNA synthetase (TyrRS) variants isolated from screens for aminoacylation of OmeY and OPG demonstrated polyspecific interaction with aromatic noncanonical amino acids (ncAAs) AcF, AzF, OmeY, OPG, and IPhe (as well as AzMF in the case of OPGRS-L6), but were not able to charge any of the aliphatic noncanonical amino acids (ncAAs) tested (FIGS. 6A and 6B, Tables 15-18). On the other hand, some Tyrosyl-tRNA synthetase (TyrRS) clones, such as epDOPARS-0.1-10 and A-BPheRS-2, showed excellent substrate specificity for their cognate noncanonical amino acids (ncAAs) DOPA and BPhe and did not charge any of the other aromatic noncanonical amino acids (ncAAs). While Tyrosyl-tRNA synthetase (TyrRS) polyspecificity clone PolyT2RS-5 efficiently aminoacylated five out of the six aromatic noncanonical amino acids (ncAAs) it was originally screened against, it did not charge the other four aromatic noncanonical amino acids (ncAAs) evaluated, nor any of the six aliphatic noncanonical amino acids (ncAAs). The Leucyl tRNA synthetase (LeuRS) polyspecificity clone PolyT2RS-7 was able to aminoacylate the same aromatic noncanonical amino acids (ncAAs) as PolyT2RS-5, as well as LysN3 and AC at low but detectable levels. Similarly, Leucyl tRNA synthetase (LeuRS) clones B-LysAlkRS-3 and B-BocKRS-2 both supported stop codon suppression with some aromatic noncanonical amino acids (ncAAs), as well as multiple aliphatic noncanonical amino acids (ncAAs). In particular, B-LysAlkRS-3 charged BocK and AC at moderate levels and LysAlk and LysN3 at high levels. Conversely, B-LysN3RS-7 did not charge any other aliphatic noncanonical amino acids (ncAAs) tested. The differences in substrate specificity between Tyrosyl-tRNA synthetase (TyrRS) mutants that were and were not screened for desired specificity or polyspecificity profiles demonstrated the power of simple modifications to the screening process for intended selectivity characteristics. Furthermore, not wishing to be bound by theory, these results may indicate that the Leucyl-tRNA synthetase (LeuRS) variants may have a stronger tendency toward polyspecific behavior when mutations are made in the active site. Further, not wishing to be bound by theory, the editing domain that is present in the E. coli Leucyl tRNA synthetase (LeuRS) but not the Tyrosyl-tRNA synthetase (TyrRS) may further support the theory that that Leucyl tRNA synthetase (LeuRS) active site is less substrate specific than the Tyrosyl-tRNA synthetase (TyrRS) active site.

### Example 10. Confirmation of Noncanonical Amino Acid Incorporation Using Mass Spectrometry

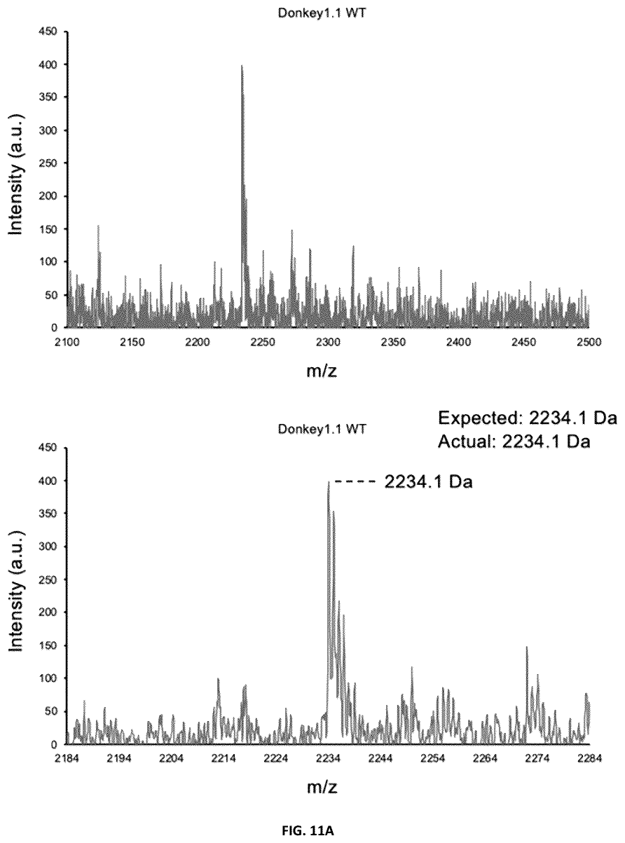

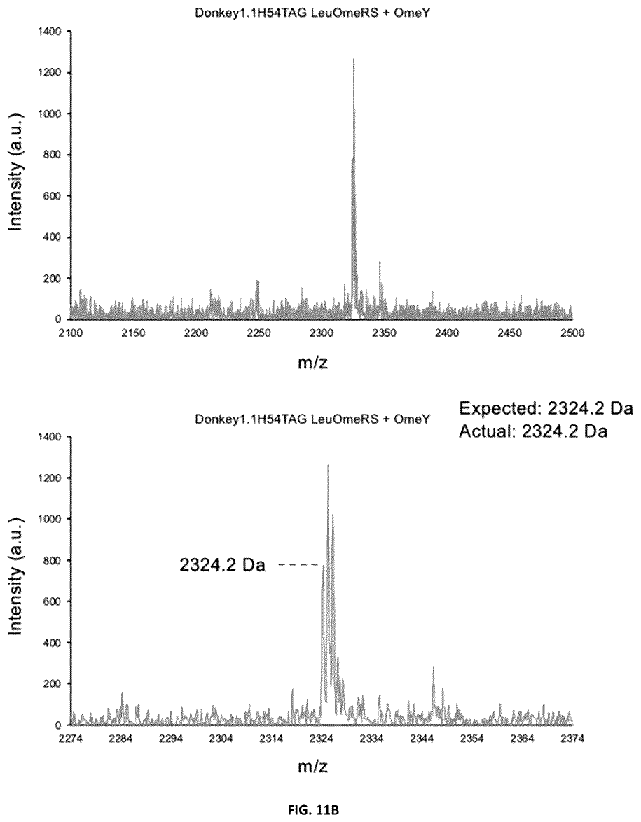

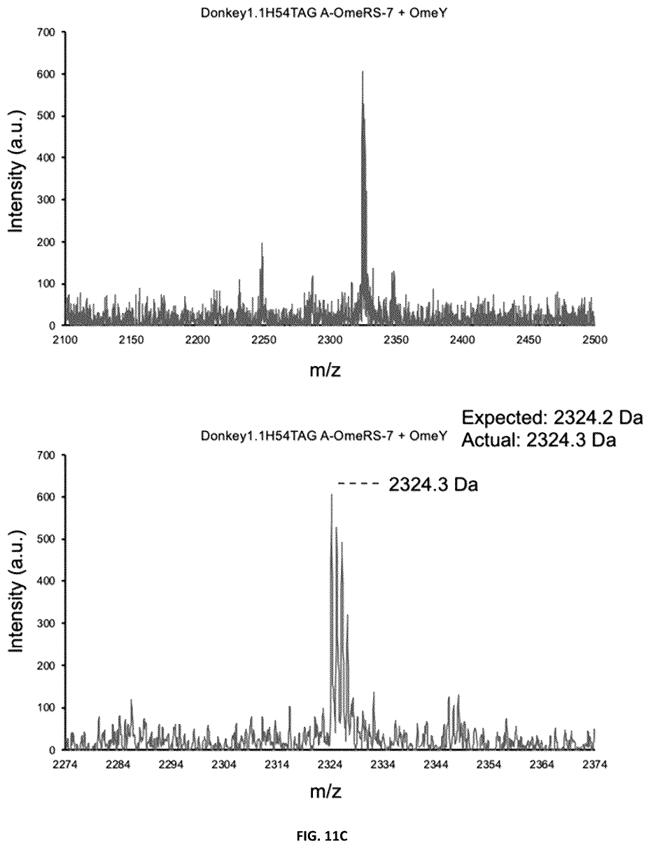

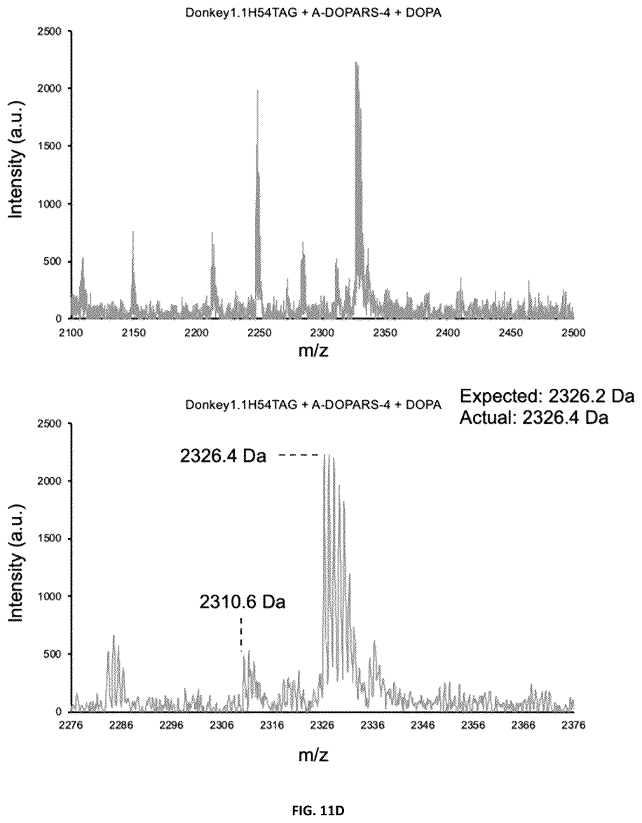

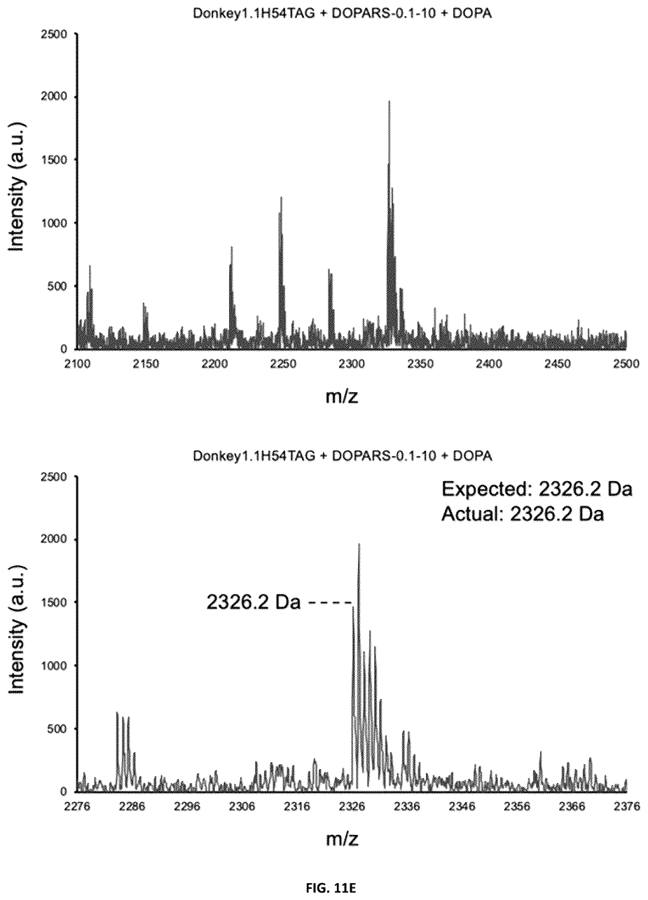

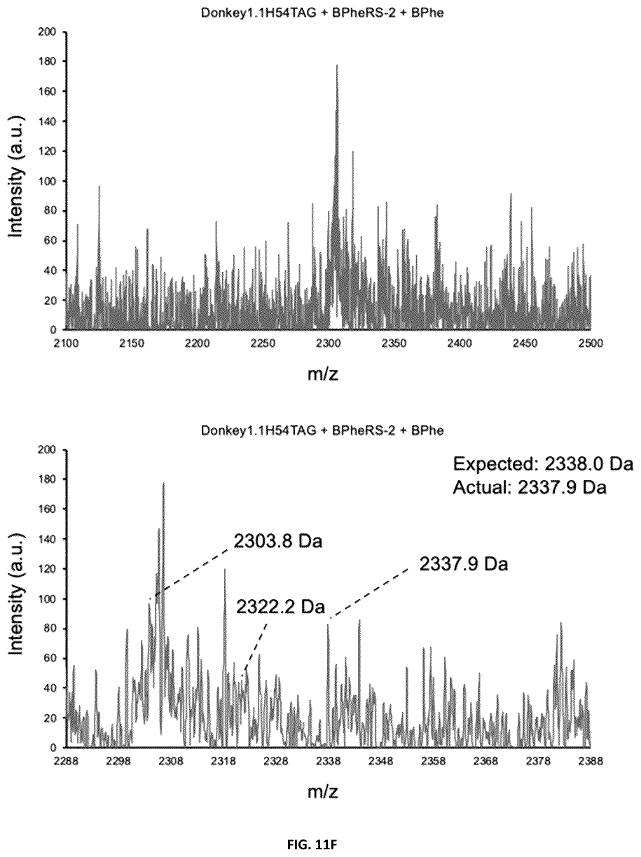

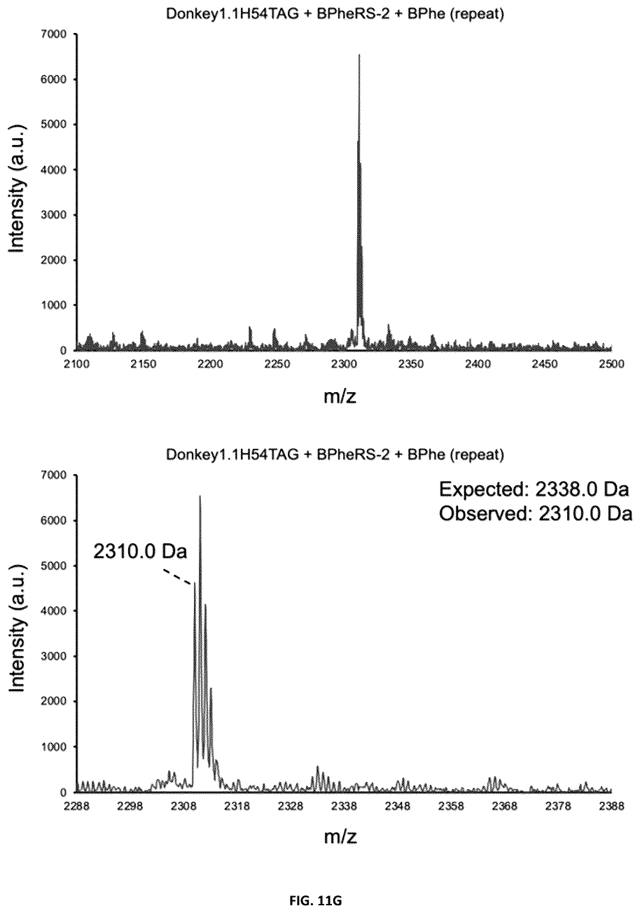

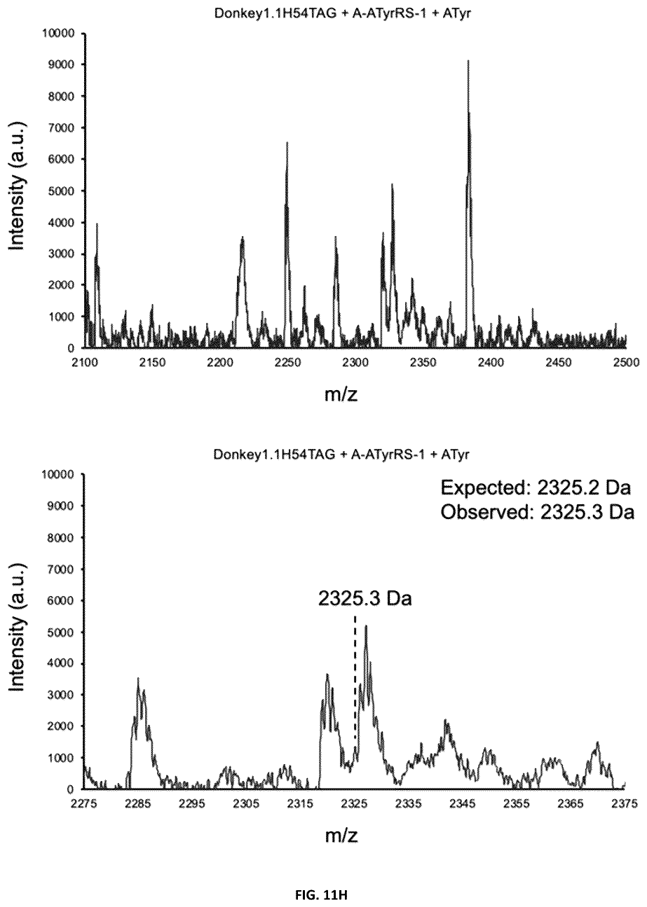

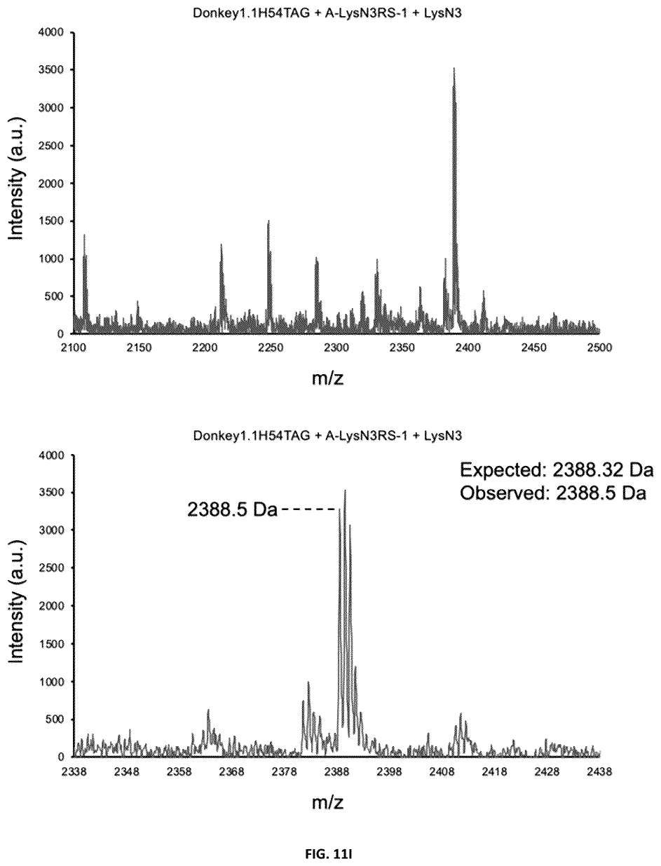

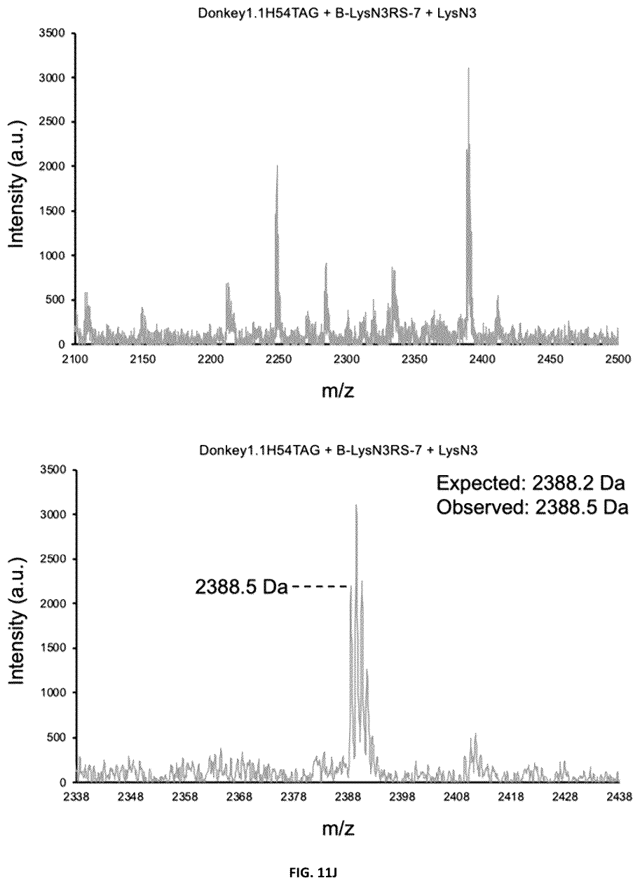

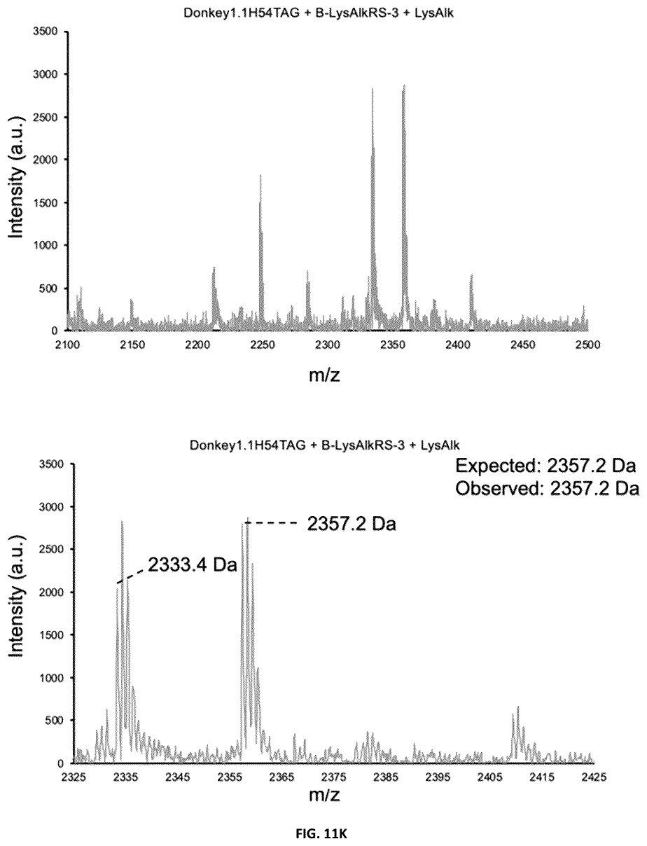

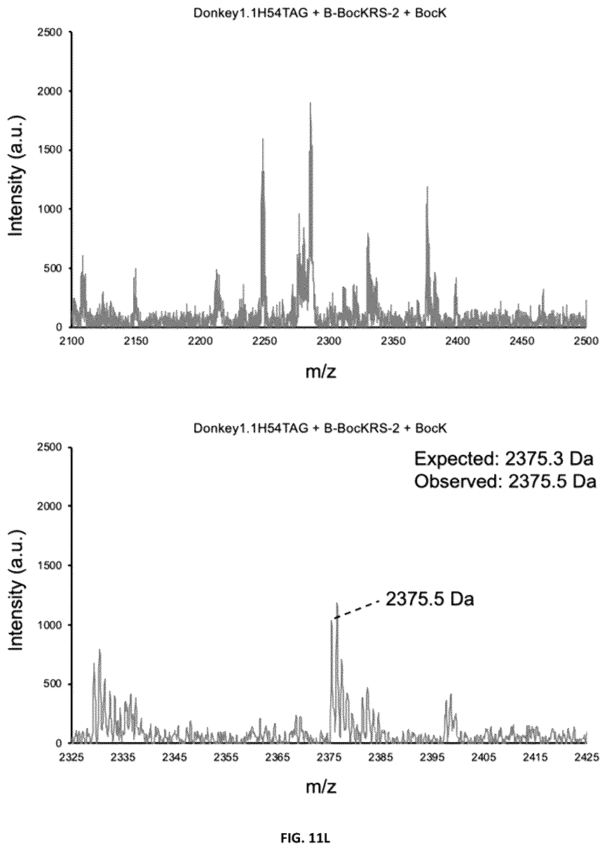

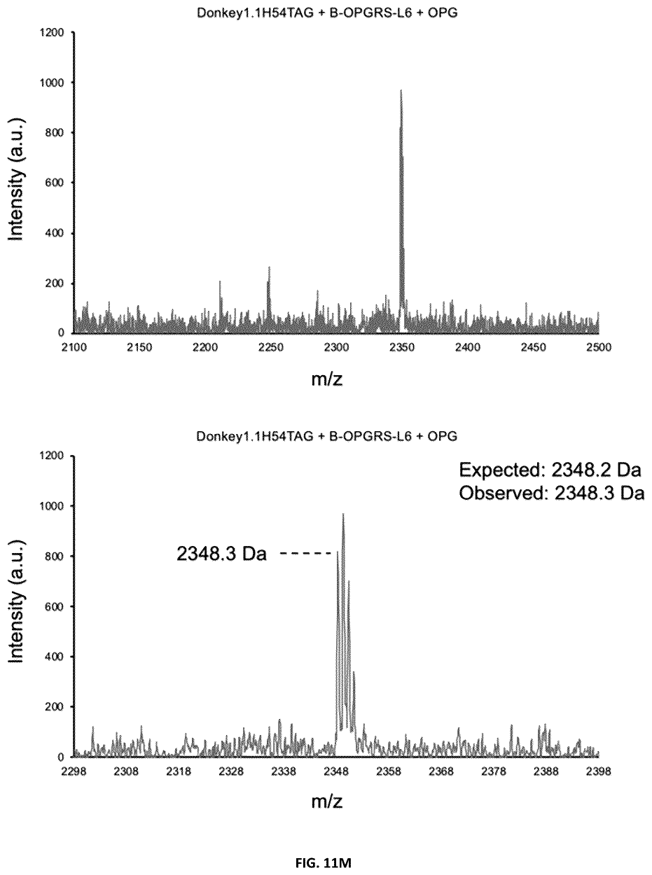

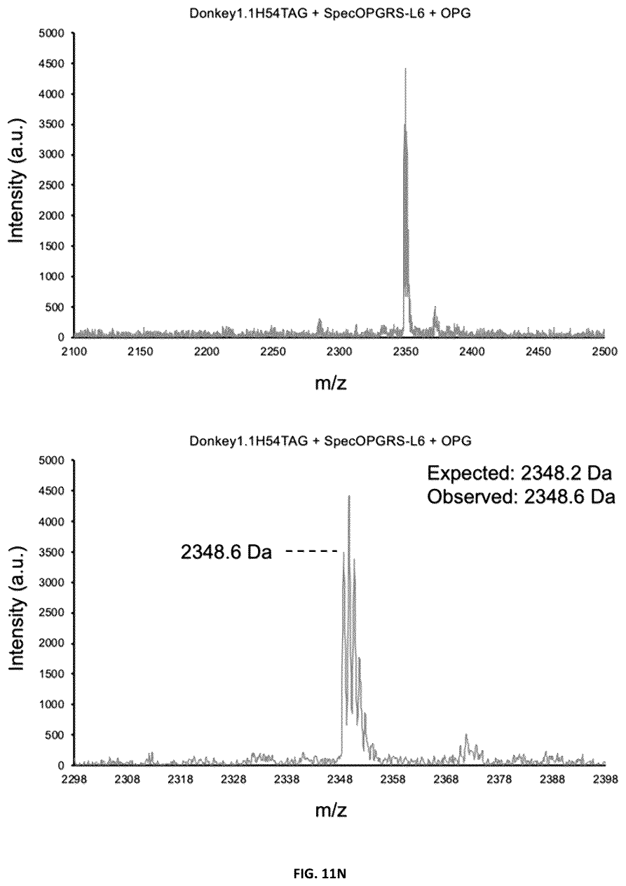

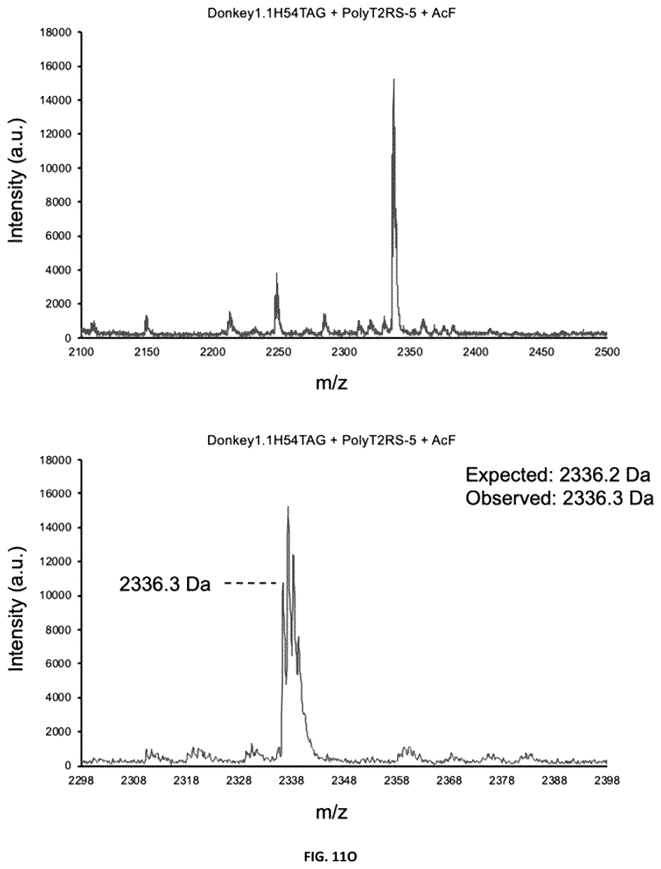

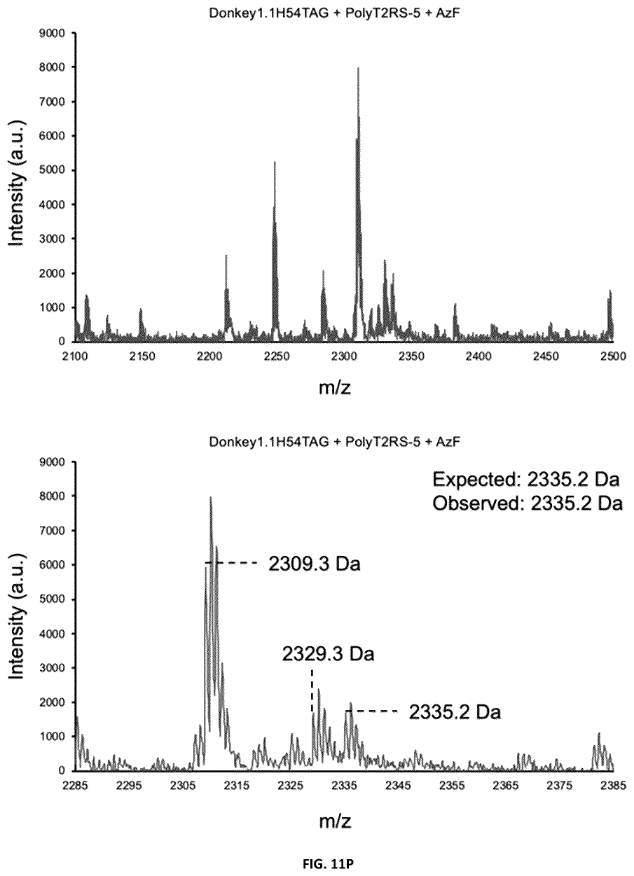

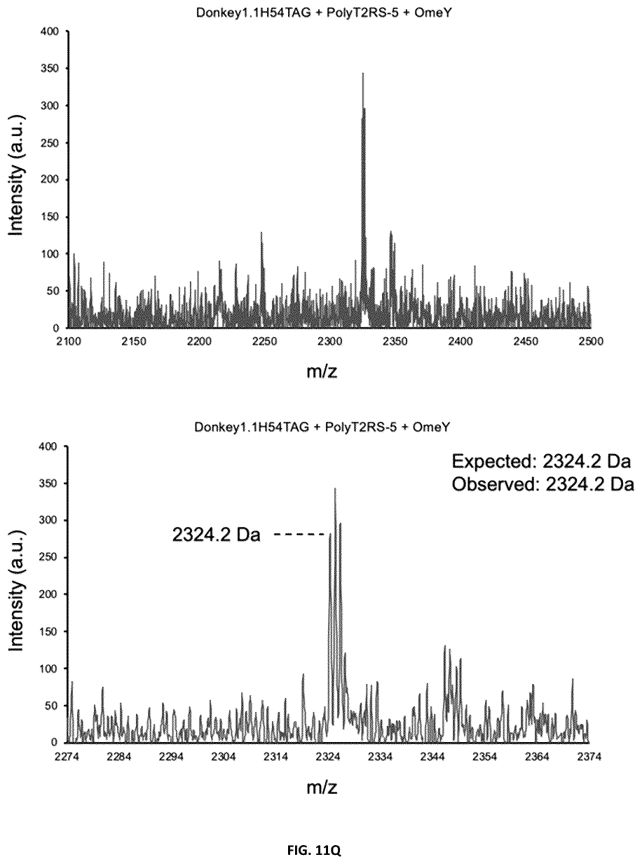

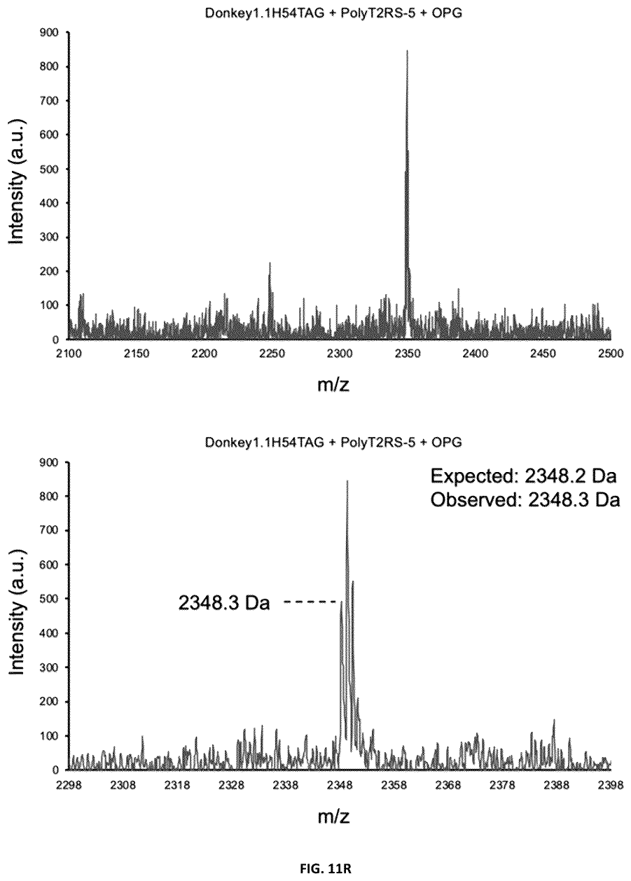

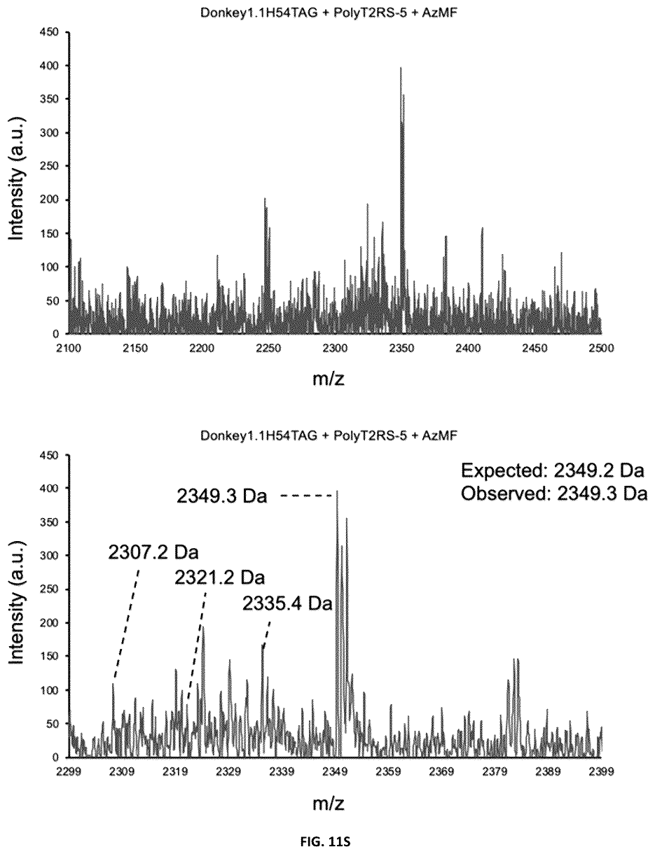

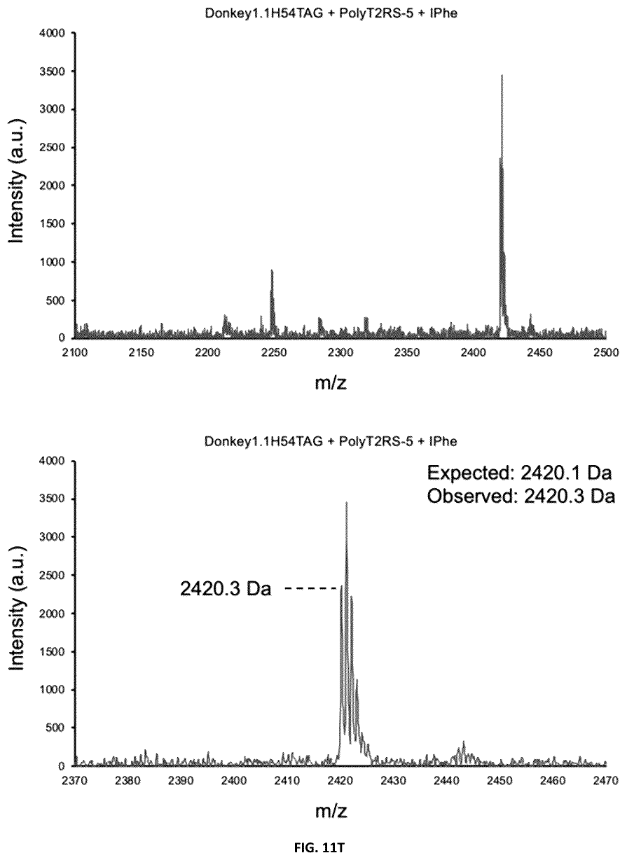

To further characterize the properties of the aminoacyl-tRNA synthetases (aaRSs), soluble ncAA-containing proteins were prepared using the aaRSs and the resulting ncAA incorporation was evaluated via MALDI mass spectrometry. Plasmids encoding each of several aminoacyl-tRNA synthetases (aaRSs) mutants were co-transformed into yeast with a secreted scFv-Fc reporter protein. Transformants were induced for secretion in rich media containing 1 mM ncAA, and resulting proteins were isolated via Protein A affinity chromatography, trypsinized, and subjected to MALDI. In most cases, detected masses for each ncAA-containing peptide were consistent with the expected masses (FIGS. 11A-11T, Table 19; MALDI provided high-accuracy mass determination of ncAA-containing polypeptides). MALDI was used due to its low sample requirements and compatibility with the glycosylated scFv-Fc reporter protein, in contrast to other commonly used MS methods. Taken as a whole, flow cytometry analysis and mass spectrometry characterizations confirmed that the aaRS libraries screened contained clones that support the incorporation of a variety of ncAAs into proteins in yeast. This validated the FACS-based discovery platform in yeast and provided opportunities to more systematically determine the range of chemically diverse ncAAs that EcTyrRS and EcLeuRS are capable of charging to suppressor tRNAs to support protein translation.

As noted above, incorporation of ncAAs was investigated via MALDI mass spectrometry of an scFv-Fc, Donkey 1.1 (H54TAG) (Islam, M.; Kehoe, H. P.; Lissoos, J. B.; Huang, M.; Ghadban, C. E.; Berumen Sanchez, G.; Lane, H. Z.; Van Deventer, J. A., Chemical Diversification of Simple Synthetic Antibodies. ACS Chem Biol 2021, 16 (2), 344-359), following expression, purification, and trypsinization (FIGS. 11A-11T). MALDI provided high-accuracy mass determination of ncAA-containing polypeptides (Fang, K. Y.; Lieblich, S. A.; Tirrell, D. A., Incorporation of Noncanonical Amino Acids into Proteins by Global Reassignment of Sense Codons. Methods Mol Biol 2018, 1798, 173-186; Duffy, N. H.; Dougherty, D. A., Preparation of translationally competent tRNA by direct chemical acylation. Org Lett 2010, 12 (17), 3776-9; Lee, J.; Schwieter, K. E.; Watkins, A. M.; Kim, D. S.; Yu, H.; Schwarz, K. J.; Lim, J.; Coronado, J.; Byrom, M.; Anslyn, E. V.; Ellington, A. D.; Moore, J. S.; Jewett, M. C., Expanding the limits of the second genetic code with ribozymes. Nat Commun 2019, 10 (1), 5097; Yang, A.; Ha, S.; Ahn, J.; Kim, R.; Kim, S.; Lee, Y.; Kim, J.; Soll, D.; Lee, H. Y.; Park, H. S., A chemical biology route to site-specific authentic protein modifications. Science 2016, 354 (6312), 623-626). MALDI was used due to its low sample requirements and compatibility with the glycosylated scFv-Fc reporter protein, in contrast to other commonly used MS methods. Protein yields in S. cerevisiae RJY100 were approximately 0.1 mg/L. Subsequent purification required for whole protein MS such as ESI further reduced protein yield. Similar to mammalian cell lines, S. cerevisiae strains are excellent for producing complex proteins, but proteins containing glycosylation sites, such as scFv-Fc use here, are glycosylated to various degrees. The deglycosylation process required the use of PNGase F, which then required further purification. Thus, the evidence presented in this Example for ncAA incorporation consists of yeast display reporter data and MALDI mass spectrometry. Below, is provided a discussion of the MALDI data presented in FIGS. 11A-11T.

Expected peptide sizes and peptide sizes that could appear due to cAA misincorporation are provided in Table 19. Expected and observed masses are reported directly on the MS spectra. Peptide masses at 2210.1, 2282.2, and 2298.2 Da were due to trypsin autolysis. The expected peptide masses of interest appeared in most samples. Both A-DOPARS-4 and DOPARS-0.1-10 had a low peak at approximately 2310 Da, which was attributed to a dehydration event that resulted in removal of one of the hydroxyl groups (FIGS. 11D and 11E). The PolyT2RS-5 sample induced in the presence of 1 mM AzF also showed a peak at 2309.3 Da indicative of degradation of AzF to APhe (expected peptide mass of 2309.2 Da, FIG. 11P). The expected peptide peak corresponding to intact AzF also appeared at 2335.2 Da. The PolyT2RS-5 sample induced in the presence of 1 mM AzMF showed the expected peptide peak at 2349.3 Da, with additional peaks at lower masses observed, presumably from the degradation (e.g., via reduction) of the azide group (FIG. 11S). Aromatic azides are often reduced in the yeast cytoplasm.

MALDI detected BPhe-containing proteins produced with BPheRS-2 (FIGS. 11F and 11G). The first MALDI spectra in FIG. 11F (which exhibited low signal-to-noise) showed a peak at 2337.9 Da, compared to an expected value of 2338.0, and additional peaks at 2303.8 and 2322.2 Da, corresponding to double and single dehydration forms of BPhe. MALDI MS was repeated for BPheRS-2, and the spectrum shown in SI FIG. 11G showed a peak at 2310.0, corresponding to the oxidized form of BPhe. With regard to MALDI of ATyr-containing proteins produced with A-ATyrRS-1, a peak at 2325.2 Da was expected corresponding to the peptide of interest and a peak at 2325.3 Da was observed. With one aaRS, it was suspected that a mixture of ncAA and tryptophan incorporation was observed: the peptide peak at 2333 Da that appears in the spectrum for B-LysAlkRS-3 is a known peptide mass that appears when tryptophan is encoded at the H54TAG position by the OTS (SI FIG. 4K). This is observation is perhaps unsurprising given that this aaRS variant was isolated using screens intended to maximize polyspecificity.

TABLE 19
Expected peptide sizes for the tryptic digest fragment
containing the H54TAG codon from the scFv-Fc form of Donkey
1.1. Both cAA misincorporation and ncAA incorporation are
included. Serine is the WT residue in the Donkey1.1
reporter, which does not contain a TAG codon at H54.
Expected peptide fragment (H54 peptide only)

| | | | Diff. from | Peptide |
|---|---|---|---|---|
| | Residue | MW (Da) | WT (Da) | Mass (Da) |
| CAAs | Serine | 105.1 | 0 | 2234.1 |
| | Alanine | 89.1 | −16 | 2218.1 |
| | Arginine | 174.2 | 69.1 | 2303.2 |
| | Asparagine | 132.1 | 27 | 2261.1 |
| | Aspartic acid | 133.1 | 28 | 2262.1 |
| | Cysteine | 121.2 | 16.1 | 2250.2 |
| | Glutamic acid | 147.1 | 42 | 2276.1 |
| | Glutamine | 146.2 | 41.1 | 2275.2 |
| | Glycine | 75.1 | −30 | 2204.1 |
| | Histidine | 155.2 | 50.1 | 2284.2 |
| | Isoleucine | 131.2 | 26.1 | 2260.2 |
| | Leucine | 131.2 | 26.1 | 2260.2 |
| | Lysine | 146.2 | 41.1 | 2275.2 |
| | Methionine | 149.2 | 44.1 | 2278.2 |
| | Phenylalanine | 165.2 | 60.1 | 2294.2 |
| | Proline | 115.1 | 10 | 2244.1 |
| | Threonine | 119.1 | 14 | 2248.1 |
| | Tryptophan | 204.2 | 99.1 | 2333.2 |
| | Tyrosine | 181.2 | 76.1 | 2310.2 |
| | Valise | 117.2 | 12.1 | 2246.2 |
| ncAAs | OmeY | 195.2 | 90.1 | 2324.2 |
| | OPG | 219.2 | 114.1 | 2348.2 |
| | AcF | 207.2 | 102.1 | 2336.2 |
| | AzF | 206.2 | 101.1 | 2335.2 |
| | AzMF | 220.2 | 115.1 | 2349.2 |
| | IPhe | 291.1 | 186 | 2420.1 |
| | BPhe | 209 | 103.9 | 2338 |
| | DOPA | 197.2 | 92.1 | 2326.2 |
| | ATyr | 196.2 | 91.1 | 2325.2 |
| | LysN3 | 259.3 | 154.2 | 2388.3 |
| | LysAlk | 228.2 | 123.1 | 2357.2 |
| | BocK | 246.3 | 141.2 | 2375.3 |
| | APhe | 180.2 | 75.1 | 2309.2 |

### Example 11. Incorporation of Crosslinkable Noncanonical Amino Acids in Proteins Produced in Yeast

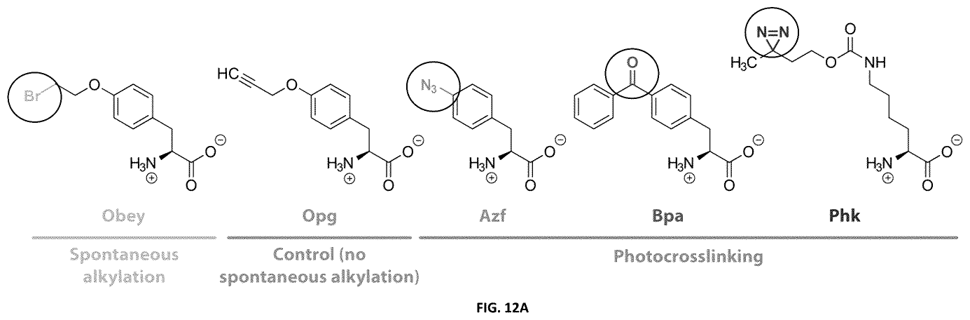

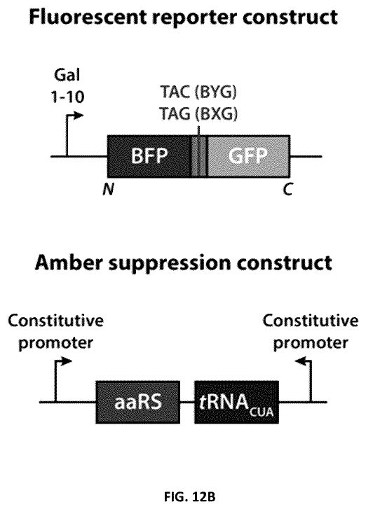

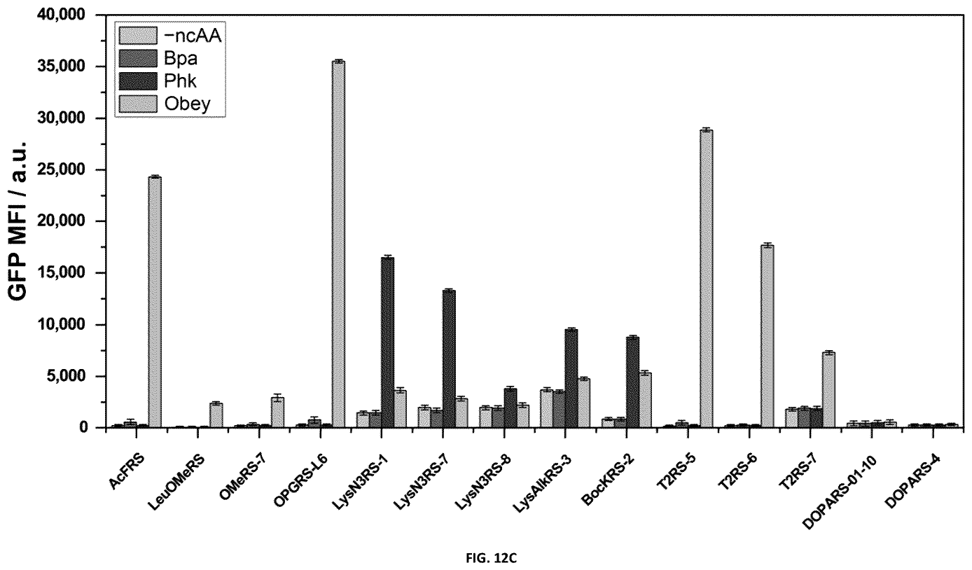

Experiments were undertaken to expand the tools available for genetically encoding crosslinkable ncAAs in proteins produced in yeast. As described in the above examples, a large number of aminoacyl-tRNA synthetase (aaRS) variants were discovered that support translation with a broad set of ncAAs. Here, several of these aaRSs were evaluated alongside previously described variants to evaluate their support of protein translation with the ncAAs O-(2-bromoethyl)tyrosine (Obey), 4-benzoyl-1-phenylalanine (Bpa), or N⁶-((2-(3-methyl-3H-diazirin-3-yl)ethoxy)carbonyl)-L-lysine (Photo-Lysine, Phk) (FIG. 12A). Although none of these ncAAs were used in any of the screening efforts described in the above Examples. several variants from the screens were nevertheless identified that supported translation at moderate to high efficiencies with Obey and Phk. Low-level translational activity was observed for Bpa with newly isolated aaRS variants, while BpaRS (see Chin, J. W.; Cropp, T. A.; Anderson, J. C.; Mukherji, M.; Zhang, Z.; Schultz, P. G., An Expanded Eukaryotic Genetic Code. Science 2003, 301 (5635), 964-967) supported moderate levels of protein translation. These observations were consistent with the notion that aaRS discovery typically leads to variants that exhibit polyspecificity. To begin to explore the potential utility of these ncAAs, they were substituted into yeast-displayed simple synthetic antibody fragments that tolerate a number of ncAA substitutions (Islam, M.; Kehoe, H. P.; Lissoos, J. B.; Huang, M.; Ghadban, C. E.; Berumen Sinchez, G.; Lane, H. Z.; Van Deventer, J. A., Chemical Diversification of Simple Synthetic Antibodies. ACS Chemical Biology 2021, 16 (2), 344-359). All incorporations of Obey, Phk, and Bpa into these constructs tested here resulted in constructs that retained antigen binding. The potential utility of these spontaneously crosslinkable and photocrosslinkable ncAAs was also investigated. For the case of Obey, a recently described anti-streptavidin peptides discovered in phage display format were displayed on the yeast surface and Obey and the control amino acid O-propargyltyrosine (Opg, FIG. 12A) were substituted into these peptides. The display and binding activities of these peptides were confirmed. These results confirmed that Obey could be incorporated into functional proteins and peptides on the yeast surface. For the cases of Phk and Bpa, photocrosslinkability on the yeast surface was investigated utilizing ncAA-substituted synthetic antibody clones and alongside AzF-substituted clones known to undergo UV-mediated crosslinking. For identical sets of crosslinking conditions on yeast, clear evidence was observed of crosslinking for Bpa and AzF, while Phk-substituted samples did not appear to undergo detectable crosslinking.

It was first sought to identify aaRSs that would support efficient incorporation of Obey, Phk, and Bpa into proteins in yeast. This was done by surveying a broad set of orthogonal translation machineries (OTSs). Here, experiments were undertaken to evaluate a diverse set aaRSs either evolved or otherwise characterized. The fourteen total synthetases (see FIG. 12C; AcFRS, LeuOMeRS, OMeRS-7, OPGRS-L6, LysN3RS-1, LysN3RS-7, LysN3RS-8, LysAlkRS-3, BocKRS-2, T2RS-5, T2RS-6, T2RS-7, DOPARS-01-10, and DOPARS-4) used in initial evaluations of translational activity included aaRSs exhibiting high readthrough activities with a range of aromatic and aliphatic ncAAs (see Islam, M.; Kehoe, H. P.; Lissoos, J. B.; Huang, M.; Ghadban, C. E.; Berumen Sinchez, G.; Lane, H. Z.; Van Deventer, J. A., Chemical Diversification of Simple Synthetic Antibodies. ACS Chemical Biology 2021, 16 (2), 344-359; Stieglitz, J. T.; Kehoe, H. P.; Lei, M.; Van Deventer, J. A., A Robust and Quantitative Reporter System To Evaluate Noncanonical Amino Acid Incorporation in Yeast. ACS Synthetic Biology 2018, 7 (9), 2256-2269; and Stieglitz, J. T.; Van Deventer, J. A., High-throughput aminoacyl-tRNA synthetase engineering for genetic code expansion in yeast. bioRxiv 2021, 2021.07.13.452272, the disclosures of which are incorporated herein by reference in their entireties for all purposes). NcAA incorporation was examined by flow cytometry using a dual fluorescent reporter, BXG, expressed intracellularly (FIG. 12B) (Potts, K. A.; Stieglitz, J. T.; Lei, M.; Van Deventer, J. A., Reporter system architecture affects measurements of noncanonical amino acid incorporation efficiency and fidelity. Molecular Systems Design & Engineering 2020, 5 (2), 573-588). Briefly, the reporter plasmid coded for blue fluorescent protein (BFP) and green fluorescent protein (GFP) connected by a small linker, where the linker contained an in-frame amber codon. In this way, detection of GFP fluorescence levels provides an initial indication of readthrough of the stop codon, with higher levels of fluorescence typically corresponding to higher levels of incorporation of the ncAA by the orthogonal translation machinery (OTS).

FIG. 12C provides an overview of the results of stop codon readthrough experiments performed with cells expressing each of the fourteen aaRSs following induction in the absence of ncAAs and in presence of 1 mM of each of the 3 ncAAs of interest. The data depicted in FIG. 12C are the median fluorescence intensities (MFI) of GFP levels determined via flow cytometry after gating the BFP-positive cells. Several aaRSs supported protein translation with Obey or Phk, while none of the aaRSs evaluated appeared to support quantitatively distinct levels of translation with Bpa. However, low levels of stop codon readthrough with Bpa were qualitatively apparent in two-dimensional dot plots. From these results, the three synthetases with the best apparent performance for Obey and Phk were chosen. For Obey, the synthetases with the highest MFI were chosen, namely AcFRS, OPGRS-L6, and T2RS-5. In the case of Phk, a similar criterion was followed and LysN3RS-1, LysN3RS-7, and BocKRS-2 were further evaluated. While LysAlkRS-3 showed higher levels of GFP in comparison to BocKRS-2, the latter was selected given the higher GFP levels of LysAlkRS-3 in the absence of ncAA, which could result from lack of specificity of the synthetase and amino acid misincorporation. Given the lack of aaRSs supporting adequate levels of translation with Bpa, the previously reported BpaRS synthetase was cloned into the OTS expression construct and its performance was characterized alongside the other aaRSs (Chin, J. W.; Cropp, T. A.; Anderson, J. C.; Mukherji, M.; Zhang, Z.; Schultz, P. G., An Expanded Eukaryotic Genetic Code. Science 2003, 301 (5635), 964-967). Both Obey and Phk exhibit high degrees of structural similarity to the ncAAs utilized in the screening and selection efforts described in the above Examples for the initial set of aaRS variants utilized here. Thus, it makes sense that variants were identified that supported translation with these ncAAs. On the other hand, Bpa contains two phenyl rings, making it distinct from the structures screened in the previous screens, which possibly explains why none of the initially evaluated aaRSs supported high levels of protein translation with Bpa.

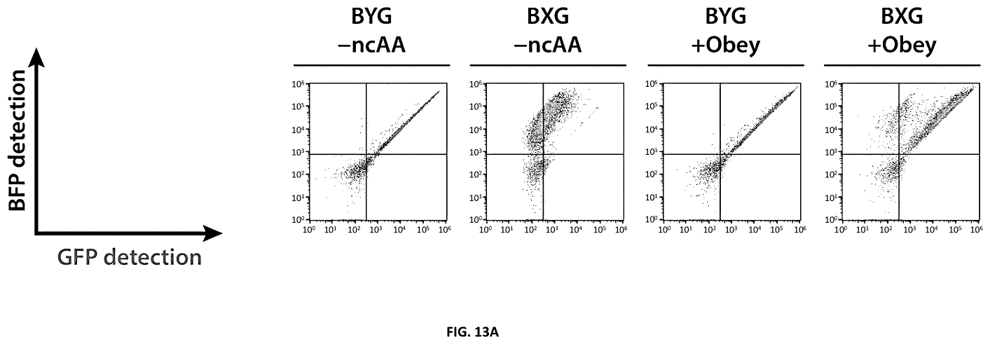

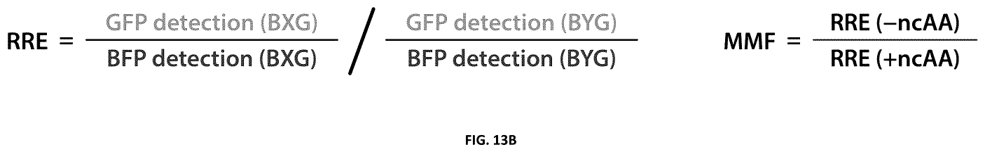

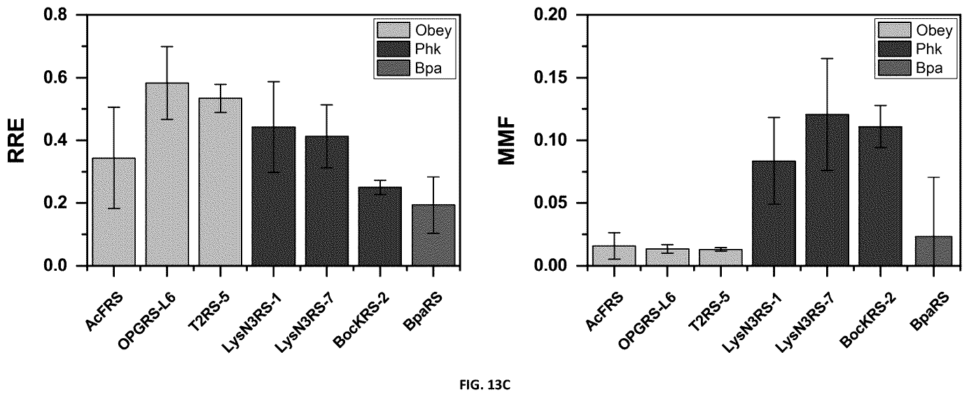

The level of incorporation was quantitated using the relative readthrough efficiency (RRE) and maximum misincorporation frequency (MMF) metrics described in Monk, J. W., et al., “Rapid and Inexpensive Evaluation of Nonstandard Amino Acid Incorporation in Escherichia coli,” ACS Synthetic Biology 2017, 6 (1), 45-54 (FIGS. 13A-13C). The BXG and BYG dual reporters were used (Potts, K. A., et al. “Reporter system architecture affects measurements of noncanonical amino acid incorporation efficiency and fidelity,” Molecular Systems Design & Engineering 2020, 5 (2), 573-588), where BYG was the “wild type” (WT) version of BXG, containing a TAC codon coding for tyrosine instead of the amber codon present in BXG. As shown on FIG. 13C, all synthetases incorporated their corresponding ncAA with RRE values near 0.20 or higher, while keeping the MFI values below 0.15, indicating a satisfactory level of efficiency of the OTS. From these results, the following synthetases showed advantageous efficiencies for the indicated ncAA based on the average RRE and MMF values: OPGRS-L6 for Obey, and LysN3RS-1 for Phk.

The above examples demonstrate the utility of an original yeast display method for screening libraries of E. coli Tyrosyl-tRNA synthetase (TyrRS) and Leucyl tRNA synthetase (LeuRS) active site mutants using fluorescence-activated cell sorting (FACS). Using this screening platform in yeast, aminoacyl-tRNA synthetases (aaRSs) that supported incorporation of noncanonical amino acids (ncAAs) were isolated that had not previously been genetically encoded in proteins in yeast: DOPA and BPhe. For a less active DOPARS clone, a single round of error-prone polymerase chain reaction (epPCR) and further screening was used to isolate aminoacyl-tRNA synthetases (aaRSs) that were better able to aminoacylate DOPA at both 0.1 and 1 mM concentrations. By introducing slight variations in the positive and negative screening methods, aminoacyl-tRNA synthetases (aaRSs) with defined specificity and polyspecificity profiles for a group of 6 aromatic noncanonical amino acid (ncAA) analogs were isolated. Further screens of a polyspecific library population for aminoacylation of several aliphatic noncanonical amino acids (ncAAs) led to isolation of highly efficient aminoacyl-tRNA synthetases (aaRSs) that could encode one or more aliphatic and aromatic noncanonical amino acids (ncAAs).

In the above examples, flow cytometry-based screens were employed to discover aaRSs exhibiting a wide range of properties for genetic code expansion in yeast. This is the first report utilizing such approaches in yeast to engineer orthogonal translation machineries (OTSs). Isolation of clones from saturation mutagenesis EcTyrRS and EcLeuRS libraries led to numerous variants supporting protein translation with a broad set of ncAAs, including DOPA, BPhe, and other ncAAs that had not previously been genetically encoded in yeast. Error-prone PCR mutagenesis of a DOPARS variant followed by increasingly stringent screening led to identification of clones that supported improved protein translation with DOPA even at reduced ncAA concentrations. The facile discovery of improved variants in a single round of mutagenesis suggests the possibility that this platform will facilitate more extensive, multi-round aaRS discovery and mutagenesis campaigns in the future—a generally underexplored route to modifying aaRS activity. Moreover, these findings highlight the strong potential to enhance OTS performance by broadly exploring aaRS diversification strategies beyond aaRS aminoacylation active sites; this observation is consistent with the findings of other recent work in this area. Further studies using random mutagenesis, deep mutational scanning, or other approaches to facilitate more comprehensive exploration of the sequence spaces surrounding known aaRSs represents a major opportunity for understanding and engineering aaRSs.

The breadth of aaRS properties accessed the above examples underscores the excellent plasticity and “evolvability” of these enzymes. Not intending to be bound by theory, the functional diversity of the mutants reported here is likely attributed primarily to the carefully controlled screening conditions, with both flow cytometry gating strategies and well-defined induction conditions playing key roles in biasing screening outcomes. There are several ways in which the findings described here could be extended further in future work. First, detailed sequence-activity relationships for the aaRSs investigated here may be attainable, especially if deep sequencing methodologies can be applied. Second, understanding the relationship between the observed translation properties reported here and underlying orthogonal translation machinery (OTS) properties (e.g., kinetic constants of aaRSs, expression levels of OTS components, and expression conditions) could lead to better understanding of how best to efficiently prepare ncAA-containing proteins in high yields and purities. Third, the availability of highly specific OTSs has the potential to facilitate genetic code expansion to include multiple ncAAs in the same protein, even when the two ncAAs of interest are similar in structure. Finally, polyspecific aaRSs have potential utility in applications in “protein medicinal chemistry,” where systematically exploring the effects of different ncAA side chains on protein properties is desirable. The findings provided in the above Examples begin to investigate the most efficient way to select sets of ncAAs that lead to tightly controlled aaRS activity using modified specificity profile screens. Overall, the availability of a high-throughput screening platform for aaRSs in yeast broadens opportunities for generating versatile aaRSs suitable for use in genetic code manipulation in yeast, mammalian cells, and other eukaryotes. Such tools are expected to facilitate dissection of basic biological and biochemical phenomena as well as myriad applications at the interface of chemical biology, synthetic biology, and protein engineering.

Tables 20-25 provide a listing of polypeptide sequences and their associated mutations identified in the above examples.

TABLE 20
LeuRS clone family descriptions.
Family Descriptions

| | |
|---|---|
| Family 1: M40G & S496T | A Leucyl-tRNA Synthetase (LeuRS) polypeptide, or a |
| | functional fragment thereof, comprising one or more amino |
| | acid substitutions selected from Q2E; M40G; T252A; S496T; |
| | a proline, glutamate, or glycine at position L41; a cysteine, |
| | leucine, histidine, or threonine at position Y499; a glycine or |
| | phenylalanine at position Y527, and a glycine or phenylalanine |
| | at position H537, or any of those amino acid substitutions |
| | relative to a wild type or reference LeuRS polypeptide |
| | sequence. |
| Family 2: S496T & H537G | A Leucyl-tRNA Synthetase (LeuRS) polypeptide, or a |
| | functional fragment thereof, comprising one or more amino |
| | acid substitutions selected from Q2E; T252A; S496T; H537G; |
| | a glycine or leucine at position M40; a proline, glutamate, or |
| | glycine at position L41; a cysteine, isoleucine, histidine, or |
| | threonine at position Y499; and a glycine or phenylalanine at |
| | position Y527, or any of those amino acid substitutions |
| | relative to a wild type or reference LeuRS polypeptide |
| | sequence. |
| Family 3: S496G & H537G | A Leucyl-tRNA Synthetase (LeuRS) polypeptide, or a |
| | functional fragment thereof, comprising one or more amino |
| | acid substitutions selected from Q2E; T252A; S496G; H537G; |
| | a glycine, serine, proline, or alanine at position M40; a |
| | threonine, proline, alanine, histidine, or glycine at position |
| | L41; a glycine, alanine, phenylalanine, cysteine, isoleucine, or |
| | threonine at position Y499; and a threonine, cysteine, glycine, |
| | histidine, isoleucine, valine, or serine at position Y527, or any |
| | of those amino acid substitutions relative to a wild type or |
| | reference LeuRS polypeptide sequence. |
| Family 4: L41P, S496G, & | A Leucyl-tRNA Synthetase (LeuRS) polypeptide, or a |
| H537G | functional fragment thereof, comprising one or more amino |
| | acid substitutions selected from Q2E; L41P; T252A; S496G; |
| | H537G; a serine, glycine, or alanine at position M40; an |
| | isoleucine, alanine, glycine, or threonine at position Y499; and |
| | a histidine, valine, or cysteine at position Y527, or any of those |
| | amino acid substitutions relative to a wild type or reference |
| | LeuRS polypeptide sequence. |
| Family 5: M40P, S496G, & | A Leucyl-tRNA Synthetase (LeuRS) polypeptide, or a |
| H537G | functional fragment thereof, comprising one or more amino |
| | acid substitutions selected from Q2E; M40P; T252A; S496G; |
| | H537G; an alanine or glycine at position L41; a cysteine, |
| | glycine, or alanine at position Y499; and an isoleucine, |
| | cysteine, or valine at position Y527, or any of those amino |
| | acid substitutions relative to a wild type or reference LeuRS |
| | polypeptide sequence. |
| Family 6: L41G & H537G | A Leucyl-tRNA Synthetase (LeuRS) polypeptide, or a |
| | functional fragment thereof, comprising one or more amino |
| | acid substitutions selected from Q2E; L41G; T252A; H537G; |
| | a glycine, alanine, or proline at position M40; a threonine or |
| | glycine at position S496; a glycine, threonine, leucine, valine, |
| | cysteine, or alanine at position Y499; and a threonine, |
| | isoleucine, cysteine, valine, or phenylalanine at position Y527, |
| | or any of those amino acid substitutions relative to a wild type |
| | or reference LeuRS polypeptide sequence. |
| Family 7: M40A & H537G | A Leucyl-tRNA Synthetase (LeuRS) polypeptide, or a |
| | functional fragment thereof, comprising one or more amino |
| | acid substitutions selected from Q2E; M40A; T252A; S496G; |
| | H537G; a proline, alanine, or glycine at position L41; a |
| | glycine, alanine, or valine at position Y499; and a threonine, |
| | cysteine, isoleucine, or histidine at position Y527, or any of |
| | those amino acid substitutions relative to a wild type or |
| | reference LeuRS polypeptide sequence. |

TABLE 21
LeuRS families clone sequences.

| Family | | Amino Acid Positions | | | | | | |
|---|---|---|---|---|---|---|---|---|
| Number | WT LeuRS | M40 | L41 | S496 | Y499 | Y527 | H537 | Target(s) |
| 1: M40G | A-LysN3RS-ori1 | G | P | T | C | Y | G | LysN3 |
| & 5496T | A-LysN3RS-4 | G | P | T | L | G | F | LysN3 |
| | A-LysN3RS-12 | G | E | T | C | Y | G | LysN3 |
| | B-PolyT2RS-7 | G | E | T | H | F | G | AcF, AzF, |
| | | | | | | | | OmeY, AzMF, |
| | | | | | | | | OPG, IPhe |
| | B-BocKRS-9 | G | G | T | T | F | G | BocK |
| 2: S496T | A-LysN3RS-ori1 | G | P | T | C | Y | G | LysN3 |
| & | A-LysN3RS-ori2 | L | P | T | I | G | G | LysN3 |
| H537G | A-LysN3RS-12 | G | E | T | C | Y | G | LysN3 |
| | B-PolyT2RS-7 | G | E | T | H | F | G | AcF, AzF, |
| | | | | | | | | OmeY, AzMF, |
| | | | | | | | | OPG, IPhe |
| | B-BocKRS-9 | G | G | T | T | F | G | BocK |
| 3: S56G | A-LysN3RS-8 | G | T | G | T | T | G | LysN3 |
| & | B-APheRS-12 | S | P | G | I | H | G | APhe |
| H537G | B-BocKRS-1 | P | A | G | G | I | G | BocK |
| | B-BocKRS-12 | A | A | G | A | C | G | BocK |
| | B-LysAlkRS-2 | S | P | G | T | V | G | LysAlk |
| | B-LysAlkRS-3 | M | H | G | A | G | G | LysAlk |
| | B-LysAlkRS-5 | P | G | G | C | C | G | LysAlk |
| | B-LysAlkRS-10 | M | L | G | F | S | G | LysAlk |
| | B-LysN3RS-1 | G | P | G | A | C | G | LysN3 |
| | B-LysN3RS-2 | P | G | G | A | V | G | LysN3 |
| | B-LysN3RS-3 | P | G | G | A | C | G | LysN3 |
| | B-LysN3RS-4 | P | G | G | C | C | G | LysN3 |
| | B-LysN3RS-6 | A | P | G | G | H | G | LysN3 |
| | B-LysN3RS-7 | S | P | G | I | H | G | LysN3 |
| | B-APheRS-12 | S | P | G | I | H | G | APhe |
| 4: L419, | B-LysAlkRS-2 | S | P | G | T | V | G | LysAlk |
| S496G, | B-LysN3RS-1 | G | P | G | A | C | G | LysN3 |
| & | B-LysN3RS-6 | A | P | G | G | H | G | LysN3 |
| H537G | B-LysN3RS-7 | S | P | G | I | H | G | LysN3 |
| 5: | B-BocKRS-1 | P | A | G | G | I | G | BocK |
| M40P, | B-LysAlkRS-5 | P | G | G | C | C | G | LysAlk |
| S498G, | B-LysN3RS-2 | P | G | G | A | V | G | LysN3 |
| & | B-LysN3RS-3 | P | G | G | A | C | G | LysN3 |
| H537G | B-LysN3RS-4 | P | G | G | C | C | G | LysN3 |
| 6: L41G | B-BocKRS-2 | A | G | S | G | T | G | BocK |
| & | B-BocKRS-9 | G | G | T | T | F | G | BocK |
| H537G | B-BocKRS-11 | P | G | S | L | T | G | BocK |
| | B-LysAlkRS-1 | A | G | S | V | I | G | LysAlk |
| | B-LysAlkRS-5 | P | G | G | C | C | G | LysAlk |
| | B-LysAlkRS-6 | A | G | S | G | T | G | LysAlk |
| | B-LysN3RS-2 | P | G | G | A | V | G | LysN3 |
| | B-LysN3RS-3 | P | G | G | A | C | G | LysN3 |
| | B-LysN3RS-4 | P | G | G | C | C | G | LysN3 |
| | B-LysN3RS-9 | A | G | S | G | T | G | LysN3 |
| 7: M40A | B-BocKRS-2 | A | G | S | G | T | G | BocK |
| & | B-BocKRS-12 | A | A | G | A | C | G | BocK |
| H537G | B-LysAlkRS-1 | A | G | S | V | I | G | LysAlk |
| | B-LysAlkRS-6 | A | G | S | G | T | G | LysAlk |
| | B-LysN3RS-6 | A | P | G | G | H | G | LysN3 |
| | B-LysN3RS-9 | A | G | S | G | T | G | LysN3 |

TABLE 22
LeuRS sequences.

| | Amino Acid Positions | | | | | | |
|---|---|---|---|---|---|---|---|
| WT LeuRS | M40 | L41 | S496 | Y499 | Y527 | H537 | Target(s) |
| A-LysN3RS-ori1 | G | P | T | C | Y | G | LysN3 |
| A-LysN3RS-ori2 | L | P | T | I | G | G | LysN3 |
| A-LysN3RS-ori3 | A | G | S | G | N | C | LysN3 |
| A-LysN3RS-4 | G | P | T | L | G | F | LysN3 |
| A-LysN3RS-6 | A | N | S | N | G | F | LysN3 |
| A-LysN3RS-8 | G | T | G | T | T | G | LysN3 |
| A-LysN3RS-12 | G | E | T | C | Y | G | LysN3 |
| A-ATyrRS-1 | L | T | A | H | S | G | ATyr |
| A-ATyrRS-12 | Q | G | S | Y | Y | H | ATyr |
| A-ATyrRS-2 | M | E | A | I | R | G | ATyr |
| B-PolyT1RS-8 | L | A | A | S | D | S | AcF, AzF, OmeY, AzMF, OPG, IPhe |
| B-SpecOPGRS-6 | L | T | A | T | T | G | OPG |
| B-PolyT2RS-7 | G | E | T | H | F | G | AcF, AzF, OmeY, AzMF, OPG, IPhe |
| B-PolyT2RS-12 | G | A | A | S | F | A | AcF, AzF, OmeY, AzMF, OPG, IPhe |
| B-APheRSRS-5 | G | V | G | T | V | L | APhe |
| B-APheRSRS-12 | S | P | G | I | H | G | APhe |
| B-BocKRS-1 | P | A | G | G | I | G | BocK |
| B-BocKRS-2 | A | G | S | G | T | G | BocK |
| B-BocKRS-3 | G | A | A | C | I | G | BocK |
| B-BocKRS-9 | G | G | T | T | F | G | BocK |
| B-BocKRS-11 | P | G | S | L | T | G | BocK |
| B-BocKRS-12 | A | A | G | A | C | G | BocK |
| B-LysAIKRS-1 | A | G | S | V | I | G | LysAlk |
| B-LysAIKRS-2 | S | P | G | T | V | G | LysAlk |
| B-LysAIKRS-3 | M | H | G | A | G | G | LysAlk |
| B-LysAIKRS-4 | G | V | G | T | V | L | LysAlk |
| B-LysAIKRS-5 | P | G | G | C | C | G | LysAlk |
| B-LysAIKRS-6 | A | G | S | G | T | G | LysAlk |
| B-LysAIKRS-10 | M | L | G | F | S | G | LysAlk |
| B-LysN3RSRS-1 | G | P | G | A | C | G | LysN3 |
| B-LysN3RSRS-2 | P | G | G | A | V | G | LysN3 |
| B-LysN3RSRS-3 | P | G | G | A | C | G | LysN3 |
| B-LysN3RSRS-4 | P | G | G | C | C | G | LysN3 |
| B-LysN3RSRS-6 | A | P | G | G | H | G | LysN3 |
| B-LysN3RSRS-7 | S | P | G | I | H | G | LysN3 |
| B-LysN3RSRS-9 | A | G | S | G | T | G | LysN3 |

TABLE 23
TyRS clone family descriptions.
Family Descriptions

| | |
|---|---|
| Family 1: Y37L & L71V | A Tyrosyl-tRNA Synthetase (TyrRS) polypeptide, or a |
| | functional fragment thereof, comprising one or more amino |
| | acid substitutions selected from Y37L; L71V; D182G; a |
| | methionine, aspartate, or asparagine at position Q179; a |
| | methionine, isoleucine, leucine, threonine, or histidine at |
| | position F183; an alanine or serine at position L186; and a |
| | threonine or gluatamate at position Q195, or any of those |
| | amino acid substitutions relative to a wild type or reference |
| | TyrRS polypeptide sequence. |
| Family 2: Y37G, L71T, & | A Tyrosyl-tRNA Synthetase (TyrRS) polypeptide, or a |
| D182S | functional fragment thereof, comprising one or more amino |
| | acid substitutions selected from Y37G; L71T; D182S; an |
| | alanine or asparagine at position Q179; a valine or leucine at |
| | position F183; an alanine or glutamate at position L186; and |
| | an isoleucine or gluatamate at position Q195, or any of those |
| | amino acid substitutions relative to a wild type or reference |
| | TyrRS polypeptide sequence. |
| Family 3: L71V & L186A | A Tyrosyl-tRNA Synthetase (TyrRS) polypeptide, or a |
| | functional fragment thereof, comprising one or more amino |
| | acid substitutions selected from L71V; L186A; a glycine, |
| | alanine, threonine, leucine, glutamate, valine, methionine, or |
| | isoleucine at position Y37; a methionine, arginine, leucine, |
| | isoleucine, glutamine, histidine, alanine, or threonine at |
| | position V72; a methionine, aspartate, proline, or valine at |
| | position Q179; a glycine or serine at position D182; a |
| | methionine, leucine, threonine, or histidine at position F183; |
| | and a threonine, valine, serine, leucine, or gluatamate at |
| | position Q195, or any of those amino acid substitutions |
| | relative to a wild type or reference TyrRS polypeptide |
| | sequence. |
| Family 4: Y37V & L71V | A Tyrosyl-tRNA Synthetase (TyrRS) polypeptide, or a |
| | functional fragment thereof, comprising one or more amino |
| | acid substitutions selected from Y37V; L71V; D182G; a |
| | threonine, leucine, or isoleucine at position V72; a valine or |
| | alanine at position Q179; a methionine or threonine at position |
| | F183; a serine or alanine at position L186; and an isoleucine or |
| | leucine at position Q195, or any of those amino acid |
| | substitutions relative to a wild type or reference TyrRS |
| | polypeptide sequence. |
| Family 5: Y37L, L71V, | A Tyrosyl-tRNA Synthetase (TyrRS) polypeptide, or a |
| L186A | functional fragment thereof, comprising one or more amino |
| | acid substitutions selected from Y37L; L71V; D182G; L186A; |
| | an aspartate or methionine at position Q179; a methionine, |
| | histidine, or threonine at position F183; and a glutamate or |
| | threonine at position Q195, or any of those amino acid |
| | substitutions relative to a wild type or reference TyrRS |
| | polypeptide sequence. |

TABLE 24
TyRS families clone sequences.

| Family | | Amino Acid Positions | | | | | | | | |
|---|---|---|---|---|---|---|---|---|---|---|
| Number | WT TyrRS | Y37 | L71 | V72 | Q179 | D182 | F183 | L186 | Q195 | Target(s) |
| 1: Y37L | A-OmeRS-1 | L | V | V | Q | G | M | A | Q | OmeY |
| & L71V | A-LysN3RS- | L | V | V | N | D | I | L | Q | LysN3 |
| | 3 | | | | | | | | | |
| | A-BPheRS-8 | L | V | V | N | D | L | S | Q | BPhe |
| | A-ATyrRS-6 | L | V | V | D | D | T | A | E | ATyr |
| | B-DOPARS- | L | V | V | M | D | H | A | T | DOPA |
| | 2 | | | | | | | | | |
| | B-PolyT2RS- | L | V | V | Q | G | M | A | Q | AcF, AzF, |
| | 2 | | | | | | | | | OmeY, |
| | | | | | | | | | | AzMF, |
| | | | | | | | | | | OPG, IPhe |
| 2: | A-BPheRS-2 | G | T | V | N | S | V | E | I | BPhe |
| Y37G, | B- | G | T | V | A | S | L | A | E | OPG |
| L71T, | SpecOPGRS- | | | | | | | | | |
| & | 3 | | | | | | | | | |
| D182S | | | | | | | | | | |
| 3: L71V | A-OmeRS-1 | L | V | V | Q | G | M | A | Q | OmeY |
| & | A-DOPARS- | E | V | V | Q | G | M | A | V | DOPA |
| L186A | 5 | | | | | | | | | |
| | A-DOPARS- | E | V | M | Q | G | M | A | T | DOPA |
| | 9 | | | | | | | | | |
| | A-APheRS-4 | V | V | V | Q | G | M | A | Q | APhe |
| | A-BPheRS-5 | M | V | R | Q | D | L | A | S | BPhe |
| | A-BPheRS-6 | V | V | L | V | D | T | A | L | BPhe |
| | A-ATyrRS-6 | L | V | V | D | D | T | A | E | ATyr |
| | B-OmeRS-4 | I | V | V | Q | G | M | A | Q | OmeY |
| | B-DOPARS- | L | V | V | M | D | H | A | T | DOPA |
| | 2 | | | | | | | | | |
| | B-APheRS-4 | E | V | M | Q | G | M | A | Q | APhe |
| | B- | A | V | V | P | S | L | A | E | OPG |
| | SpecOPGRS- | | | | | | | | | |
| | 1 | | | | | | | | | |
| | B- | T | V | H | Q | G | M | A | Q | OPG |
| | SpecOPGRS- | | | | | | | | | |
| | 2 | | | | | | | | | |
| | B- | G | V | Q | Q | S | T | A | E | OPG |
| | SpecOPGRS- | | | | | | | | | |
| | 4 | | | | | | | | | |
| | B- | T | V | A | Q | G | M | A | Q | OPG |
| | SpecOPGRS- | | | | | | | | | |
| | 9 | | | | | | | | | |
| | B- | T | V | T | Q | G | M | A | Q | OPG |
| | SpecOPGRS- | | | | | | | | | |
| | 12 | | | | | | | | | |
| | B-Poly T2RS- | L | V | V | Q | G | M | A | Q | AcF, AzF, |
| | 2 | | | | | | | | | OmeY, |
| | | | | | | | | | | AzMF, |
| | | | | | | | | | | OPG, IPhe |
| | B-PolyT2RS- | V | V | V | Q | G | M | A | Q | AcF, AzF, |
| | 4 | | | | | | | | | OmeY, |
| | | | | | | | | | | AzMF, |
| | | | | | | | | | | OPG, IPhe |
| | B-Poly T2RS- | V | V | I | Q | G | M | A | Q | AcF, AzF, |
| | 5 | | | | | | | | | OmeY, |
| | | | | | | | | | | AzMF, |
| | | | | | | | | | | OPG, IPhe |
| | B-Poly T2RS- | I | V | V | Q | G | M | A | Q | AcF, AzF, |
| | 6 | | | | | | | | | OmeY, |
| | | | | | | | | | | AzMF, |
| | | | | | | | | | | OPG, IPhe |
| | B-OPGRS- | V | V | T | Q | G | M | A | Q | OPG |
| | 1H | | | | | | | | | |
| 4: Y37V | A-LysN3RS- | V | V | V | A | D | T | S | I | LysN3 |
| & L71V | 1 | | | | | | | | | |
| | A-APheRS-4 | V | V | V | Q | G | M | A | Q | APhe |
| | A-BPheRS-6 | V | V | L | V | D | T | A | L | BPhe |
| | B-PolyT2RS- | V | V | V | Q | G | M | A | Q | AcF, AzF, |
| | 4 | | | | | | | | | OmeY, |
| | | | | | | | | | | AzMF, |
| | | | | | | | | | | OPG, IPhe |
| | B-PolyT2RS- | V | V | I | Q | G | M | A | Q | AcF, AzF, |
| | 5 | | | | | | | | | OmeY, |
| | | | | | | | | | | AzMF, |
| | | | | | | | | | | OPG, IPhe |
| | B-OPGRS- | V | V | T | Q | G | M | A | Q | OPG |
| | 1H | | | | | | | | | |
| 5: | A-OmeRS-1 | L | V | V | Q | G | M | A | Q | OmeY |
| Y37L, | A-ATyrRS-6 | L | V | V | D | D | T | A | E | ATyr |
| L71V, | B-DOPARS- | L | V | V | M | D | H | A | T | DOPA |
| L186A | 2 | | | | | | | | | |
| | B-Poly T2RS- | L | V | V | Q | G | M | A | Q | AcF, AzF, |
| | 2 | | | | | | | | | OmeY, |
| | | | | | | | | | | AzMF, |
| | | | | | | | | | | OPG, IPhe |

TABLE 25
TyRS sequences. Wild type amino acids are italicized.

| | Amino Acid Positions | | | | | | | | |
|---|---|---|---|---|---|---|---|---|---|
| WT TyrRS | Y37 | L71 | V72 | Q179 | D182 | F183 | L186 | Q195 | Target(s) |
| A-OmeRS-1 | L | V | V | Q | G | M | A | Q | OmeY |
| A-OmeRS-3 | G | R | V | P | S | P | L | S | OmeY |
| A-OmeRS-7 | L | L | V | Q | G | M | A | Q | OmeY |
| A-DOPARS-1 | E | L | V | Q | G | M | A | T | DOPA |
| A-DOPARS-2 | L | L | V | G | D | T | V | L | DOPA |
| A-DOPARS-3 | T | L | V | M | D | N | L | V | DOPA |
| A-DOPARS-4 | Q | M | V | S | D | T | V | I | DOPA |
| A-DOPARS-5 | E | V | V | Q | G | M | A | V | DOPA |
| A-DOPARS-7 | T | I | V | M | D | M | A | K | DOPA |
| A-DOPARS-9 | E | V | M | Q | G | M | A | T | DOPA |
| A-LysN3RS-1 | V | V | V | A | D | T | S | I | LysN3 |
| A-LysN3RS-2 | A | M | V | Q | D | T | V | V | LysN3 |
| A-LysN3RS-3 | L | V | V | N | D | I | L | Q | LysN3 |
| A-LysN3RS-5 | I | L | V | Q | D | G | L | Q | LysN3 |
| A-LysN3RS-9 | I | L | V | Q | D | V | A | L | LysN3 |
| A-LysN3RS- | L | D | V | L | D | T | L | V | LysN3 |
| 11 | | | | | | | | | |
| A-APheRS-1 | I | L | V | Q | G | M | A | Q | APhe |
| A-APheRS-4 | V | V | V | Q | G | M | A | Q | APhe |
| A-APheRS-6 | G | L | V | T | D | T | S | M | APhe |
| A-APheRS-8 | E | L | V | Q | G | M | A | S | APhe |
| A-APheRS-10 | T | V | S | N | D | I | L | G | APhe |
| A-BPheRS-1 | E | I | V | L | S | I | S | E | BPhe |
| A-BPheRS-2 | G | T | V | N | S | V | E | I | BPhe |
| A-BPheRS-3 | A | L | V | P | D | T | L | A | BPhe |
| A-BPheRS-5 | M | V | R | Q | D | L | A | S | BPhe |
| A-BPheRS-6 | V | V | L | V | D | T | A | L | BPhe |
| A-BPheRS-8 | L | V | V | N | D | L | S | Q | BPhe |
| A-ATyrRS-4 | L | L | V | M | D | Q | A | S | ATyr |
| A-ATyrRS-6 | L | V | V | D | D | T | A | E | ATyr |
| A-ATyrRS-7 | L | M | V | N | D | I | S | L | ATyr |
| A-ATyrRS-10 | E | V | V | H | D | V | L | I | ATyr |
| B-OmeRS-3 | I | L | V | Q | G | M | A | Q | OmeY |
| B-OmeRS-4 | I | V | V | Q | G | M | A | Q | OmeY |
| B-OmeRS-6 | I | L | V | M | G | M | A | Q | OmeY |
| B-DOPARS-2 | L | V | V | M | D | H | A | T | DOPA |
| B-DOPARS-4 | T | I | V | M | D | M | A | K | DOPA |
| B-DOPARS-5 | H | L | V | M | D | L | A | G | DOPA |
| B-DOPARS-9 | Q | L | V | H | S | D | A | V | DOPA |
| B-DOPARS- | Q | V | V | Q | D | H | L | V | DOPA |
| 11 | | | | | | | | | |
| B-DOPARS- | T | L | V | T | D | N | V | T | DOPA |
| 14 | | | | | | | | | |
| B-APheRS-1 | I | L | V | Q | G | M | A | Q | APhe |
| B-APheRS-4 | E | V | M | Q | G | M | A | Q | APhe |
| B-APheRS-11 | D | L | V | Q | D | A | A | H | APhe |
| B-ATyrRS-5 | I | L | V | E | D | T | L | S | ATyr |
| B-PolyT1RS-2 | L | L | V | Q | G | M | A | Q | AcF, AzF, OmeY, |
| | | | | | | | | | AzMF, OPG, IPhe |
| B-PolyT1RS-7 | V | L | V | Q | G | M | A | Q | AcF, AzF, OmeY, |
| | | | | | | | | | AzMF, OPG, IPhe |
| B-PolyT1RS- | T | L | V | Q | G | M | A | Q | AcF, AzF, OmeY, |
| 12 | | | | | | | | | AzMF, OPG, IPhe |
| B- | A | V | V | P | S | L | A | E | OPG |
| SpecOPGRS-1 | | | | | | | | | |
| B- | T | V | H | Q | G | M | A | Q | OPG |
| SpecOPGRS-2 | | | | | | | | | |
| B- | G | T | V | A | S | L | A | E | OPG |
| SpecOPGRS-3 | | | | | | | | | |
| B- | G | V | Q | Q | S | T | A | E | OPG |
| SpecOPGRS-4 | | | | | | | | | |
| B- | T | L | V | E | S | T | L | L | OPG |
| SpecOPGRS-7 | | | | | | | | | |
| B- | T | V | A | Q | G | M | A | Q | OPG |
| SpecOPGRS-9 | | | | | | | | | |
| B- | T | V | T | Q | G | M | A | Q | OPG |
| SpecOPGRS- | | | | | | | | | |
| 12 | | | | | | | | | |
| B-PolyT2RS-2 | L | V | V | Q | G | M | A | Q | AcF, AzF, OmeY, |
| | | | | | | | | | AzMF, OPG, IPhe |
| B-PolyT2RS-4 | V | V | V | Q | G | M | A | Q | AcF, AzF, OmeY, |
| | | | | | | | | | AzMF, OPG, IPhe |
| B-PolyT2RS-5 | V | V | I | Q | G | M | A | Q | AcF, AzF, OmeY, |
| | | | | | | | | | AzMF, OPG, IPhe |
| B-PolyT2RS-6 | I | V | V | Q | G | M | A | Q | AcF, AzF, OmeY, |
| | | | | | | | | | AzMF, OPG, IPhe |
| B-OPGRS-1H | V | V | T | Q | G | M | A | Q | OPG |
| B-OPGRS-6L | V | I | V | Q | G | M | A | Q | OPG |
| B-OPGRS-8L | T | L | V | Q | G | M | A | Q | OPG |
| B-OPGRS- | M | L | V | Q | S | R | L | Q | OPG |
| 10L | | | | | | | | | |

### Methods of the Examples

The following materials and methods were employed in the above examples.

All restriction enzymes used for molecular biology were from New England Biolabs (NEB). Synthetic oligonucleotides for cloning and sequencing were purchased from Eurofins Genomics or GENEWIZ. All sequencing was performed by Eurofins Genomics (Louisville, KY) or Quintara Biosciences (Cambridge, MA). Epoch Life Science GenCatch™ Plasmid DNA Mini-Prep Kits were used for plasmid DNA purification from E. coli. Yeast chemical competent cells and subsequent transformations were prepared using Zymo Research Frozen-EZ Yeast Transformation II kits. Noncanonical amino acids were purchased from the indicated companies: p-acetyl-L-phenylalanine (SynChem), p-azido-L-phenylalanine (Chem-Impex International), O-methyl-L-tyrosine (Chem-Impex International), p-propargyloxy-L-phenylalanine (Iris Biotech), 4-azidomethyl-L-phenylalanine (SynChem), 4-iodo-L-phenylalanine (AstaTech), 3,4-dihydroxy-L-phenylalanine (Alfa Aesar), 4-borono-L-phenylalanine (Acros Organics), 3-Amino-L-tyrosine (Bachem), 4-Amino-L-phenylalanine (Bachem), (S)-2-amino-6-((2-azidoethoxy)carbonylamino)hexanoic acid (Iris Biotech), (S)-2-amino-6-(((prop-2-yn-1-yloxy)carbonyl)amino)hexanoic acid (AstaTech), N^{ε}-Boc-L-lysine (Chem-Impex International), N^{ε}-azido-L-lysine (Chem-Impex International), N^{ε}-dimethyl-L-lysine (Chem-Impex International), and L-α-aminocaprylic acid (Acros Organics). Table 26 provides a list of noncanonical amino acids (NcAAs) used in the Examples.

TABLE 26
NcAA names, abbreviations, suppliers, and CAS numbers.

| # | Name | Abbreviation | Supplier | CAS |
|---|---|---|---|---|
| 1 | O-methyl-L-tyrosine | OmeY | Chem-Impex International | 6230-11-1 |
| 2 | p-acetyl-L-phenylalanine | AcF | SynChem | 22888-49-9′ |
| 3 | p-azido-L-phenylalanine | AzF | Chem-Impex International | 33173-53-4 |
| 4 | p-propargyloxy-L-phenylalanine | OPG | Iris Biotech | 610794-20-2 |
| 5 | 4-azidomethyl-L-phenylalanine | AzMF | SynChem | 1446772-80-0 |
| 6 | 4-borono-L-phenylalanine | BPhe | Acros Organics | 76410-58-7 |
| 7 | 3,4-dihydroxy-L-phenylalanine | DOPA | Alfa Aesar | 59-92-7 |
| 8 | 4-iodo-L-phenylalanine | IPhe | AstaTech | 24250-85-9 |
| 9 | L-a-aminocaprylic acid | AC | Acros Organics | 644-90-6* |
| 10 | N^{ε}-azido-L-lysine | AzK | Chem-Impex International | 1454334-76-9 |
| 11 | 3-Amino-L-tyrosine | ATyr | Bachem | 23279-22-3 |
| 12 | 4-Amino-L-phenylalanine | APhe | Bachem | 62040-55-5 |
| 13 | N^{ε},N^{ε}-dimethyl-L-lysine | DMK | Chem-Impex International | 2259-86-1 |
| 14 | N^{ε}-Boc-L-lysine | BocK | Chem-Impex International | 2418-95-3 |
| 15 | (S)-2-amino-6-((2-azidoethoxy)carbonylamino)hexanoic acid | LysN3 | Iris Biotech | 1994331-17-7 |
| 16 | (2S)-2-amino-6-(((prop-2-yn-1-yloxy)carbonyl)amino)hexanoic acid | LysAlk | AstaTech | 1428330-91-9 |

*The CAS number supplied is for the DL racemic mixture. Indicated ncAA concentrations were for L isomer only.

### Media Preparation and Yeast Strain Construction

The preparation of liquid and solid media was performed as described in Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269. Unless otherwise noted, all SD-SCAA and SG-SCAA media used here were prepared without tryptophan (TRP), leucine (LEU) or uracil (URA). The strain RJY100 was constructed using standard homologous recombination approaches as described in Van Deventer, J. A., Kelly, R. L., Rajan, S., Wittrup, K. D., and Sidhu, S. S. (2015) A switchable yeast display/secretion system, Protein Eng Des Sel 28, 317-325.

### Preparing Noncanonical Amino Acid Liquid Stocks

All noncanonical amino acid (ncAA) stocks were prepared at a final concentration of 50 mM concentration of the L-isomer. DI water was added to the solid noncanonical amino acid (ncAA) to approximately 90% of the final volume needed to make the stock, and 6.0 N NaOH was used as needed to fully dissolve the noncanonical amino acid (ncAA) powder in the water. Water was added to the final volume and the solution was sterile filtered through a 0.2 micron filter. OmeY was pH adjusted to 7 prior to sterile filtering. No pH adjustment was performed unless otherwise noted. Filtered solutions were stored at 4° C. for up to four weeks for less labile noncanonical amino acids (ncAAs); for more labile noncanonical amino acids (ncAAs) (AzF, BPHe, DOPA), 50 mM stocks were made immediately prior to induction.

### Reporter Plasmid Construction

The pCTCON2-FAPB2.3.6 and pCTCON2-FAPB2.3.6L1TAG reporter constructs are described in Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269. pCTCON2-FAPB2.3.6 was used as a wildtype (WT) control to compare TAG-containing samples against and was not used for library construction or sorting.

Tyrosyl-tRNA Synthetase (TyrRS) and Leucyl tRNA Synthetase (LeuRS) Library Construction and Characterization

The previously reported pRS315-AcFRS plasmid with additional NcoI and NdeI restriction enzyme recognition sites flanking the aminoacyl-tRNA synthetase (aaRS) gene (Potts, K. A., Stieglitz, J. T., Lei, M., Van Deventer, J. A. (2020) Reporter system architecture affects measurements of noncanonical amino acid incorporation efficiency and fidelity, Mol. Syst. Des. Eng. 5, 573-588) was further modified by replacing the ampicillin resistance marker with a kanamycin/neomycin resistance marker. Restriction enzyme sites XmaI and AvrII were introduced on either side of the ampicillin resistance marker via Quick Change, then the kanamycin/neomycin marker was amplified from pREP4 with primers containing 30 bases of overlap with pRS315-AcFRS (containing the NcoI and NdeI sites) that had been double digested with XmaI and AvrII. The PCR amplified kanamycin/neomycin gene was then cloned into the digest pRS315-AcFRS vector via Gibson assembly. Gibson assembly reactions were transformed into chemically competent E. coli and plated on LB plates with kanamycin at a final concentration of 34 μg/mL. Colonies were inoculated in selective liquid media, grown to saturation, miniprepped, and sequenced. The resulting plasmid was named pRS315-KanR-AcFRS. Additional PCRs of the kanamycin/neomycin resistance gene were done to remove a NcoI restriction enzyme site from the gene and were cloned into the pRS315-KanR-AcFRS plasmid that was double digested with XmaI and AvrII. The resulting plasmid was sequence verified and named pRS315-KanRmod-AcFRS. The plasmid pRS315-EcLeuRS containing the E. coli leucyl-tRNA synthetase (LeuRS) with a T252A mutation in the editing domain is described in Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269. The entire Leucyl tRNA synthetase (LeuRS) gene and cognate tRNA as well as the constitutive promoters for each gene were PCR amplified from pRS315-EcLeuRS and cloned into pRS315-KanRmod-AcFRS that was double digested with SacI and PstI via Gibson assembly. The resulting plasmid was sequence verified and named pRS315-KanRmod-EcLeuRS.

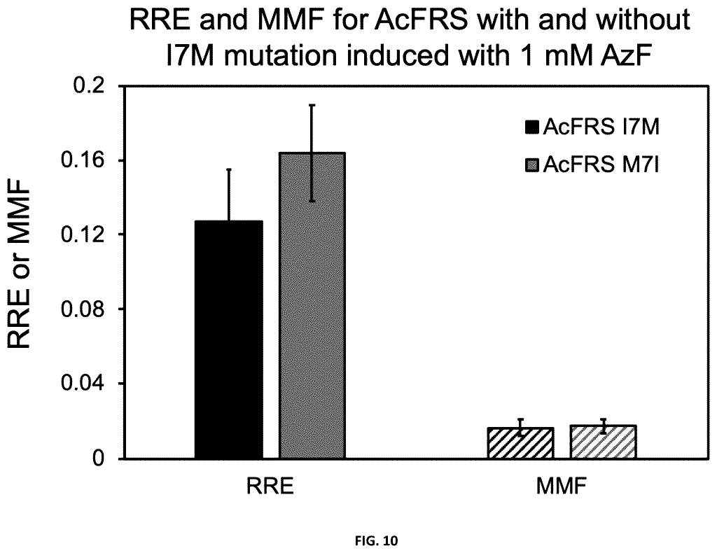

Primers containing degenerate codons were used to amplify the aminoacyl-tRNA synthetase (aaRS) genes from parent plasmids pRS315-KanRmod-EcLeuRS (LeuRS library) or pRS315-KanRmod-AcFRS (TyrRS library). Seven positions in the Tyrosyl-tRNA synthetase (TyrRS) active site were chosen for mutation: Y37, L71, Q179, N182, F183, L186, and Q195 (Tables 1 and 2). A separate primer with only the WT tyrosine codon at position Y37 was also used. The AcFRS gene contained a preexisting D165G mutation. An additional mutation, I7M, was inadvertently introduced when a primer containing that mutation was received and used for PCR amplification of the gene. However, a side-by-side comparison of AcFRS with and without the I7M mutations showed that the activity of AcFRS was not significantly affected by the presence of the mutation (FIG. 10). pRS315-KanRmod-AcFRS was double digested with restriction enzymes NcoI and NdeI and the PCR-amplified Tyrosyl-tRNA synthetase (TyrRS) gene fragments were concentrated using Pellet Paint® NF Co-precipitant according to the manufacturer's protocols. Similarly, seven positions in the Leucyl tRNA synthetase (LeuRS) active site were chosen for mutation: M40, L41, T252, S496, Y499, Y527, and H537, with only an alanine mutation at position T252 (Tables 3-5). pRS315-KanRmod-LeuRS was double digested with restriction enzymes NcoI and NdeI and the PCR-amplified Leucyl tRNA synthetase (LeuRS) gene fragments were concentrated using Pellet Paint® NF Co-precipitant according to the manufacturer's protocols. A control DNA sample for each aminoacyl-tRNA synthetase (aaRS) library that contained only double digested vector and no insert DNA was prepared similarly. Electrocompetent S. cerevisiae RJY100 were prepared as described in Van Deventer, J. A., and Wittrup, K. D. (2014) Yeast surface display for antibody isolation: library construction, library screening, and affinity maturation, Methods Mol Biol 1131, 151-181, and electroporation protocols for transforming the library DNA were also followed. Electroporated cells were plated for transformation efficiency on SD-SCAA (-TRP-LEU-URA) and recovered in SD-SCAA (-TRP-LEU-URA) supplemented with penicillin-streptomycin. Dilutions plated on solid media to evaluate transformation efficiency were grown at 30° C. for 3-4 days and colonies were counted and averaged in quadrants to approximate the number of individual transformants. The remainder of the libraries were grown at 30° C. to saturation, then passaged to 1 L SD-SCAA (-TRP-LEU-URA) and grown at 30° C. overnight. The 1 L cultures were centrifuged at maximum speed for 30 min and the supernatant was decanted. The remainder of the cell pellets were resuspended in 60% glycerol, aliquoted to cryogenic vials, and stored at −80° C. A portion of each library was passaged separately in 5 mL SD-SCAA (-TRP-LEU-URA) cultures to evaluate the naïve library on a flow cytometer and to isolate the aminoacyl-tRNA synthetase (aaRS) plasmids from the cells and send for sequence verification (see below for details).

Error-Prone PCR (epPCR) Library Construction and Characterization

E. coli Tyrosyl-tRNA synthetase (TyrRS) mutant A-DOPARS-8 was used as a template for error-prone polymerase chain reaction (epPCR). Error-prone polymerase chain reaction (epPCR) was performed by combining 5 μL 10× ThermoPol Buffer, 1 μL 10 mM dNTP, 5 μL 20 μM or 100 μM dPTP, 5 μL 20 μM or 100 μM 8-oxo-dGTP, 1 μL Taq polymerase, 1 μL DNA template (1 ng total), and 2.5 μL of each forward and reverse primer at 10 μM to amplify across the entire aminoacyl-tRNA synthetase (aaRS) gene, as well as 27 μL sterile water to bring the total volume to 50 μL. Two concentrations (20 μM or 100 μM) of mutagenic dNTPs were used to vary the number of mutations made across the aminoacyl-tRNA synthetase (aaRS). Reactions were run on the thermal cycler at 95° C. for 500 s followed by 16 cycles of 95° C. for 45 s, 60° C. for 30 s, 72° C. for 135 s. Once cycles were complete, samples underwent a 10 min 72° C. final extension and hold at 4° C. until they were removed from the thermal cycler.

Following PCR with mutagenic dNTPs, each gene was amplified again via PCR at a higher volume to prepare enough DNA for electroporation into yeast. PCR was performed by combining 20 μL 10× ThermoPol Buffer, 4 μL 10 mM dNTP, 4 μL Taq polymerase, 10 μL error-prone polymerase chain reaction (epPCR)-mutated DNA template, and 2 μL of each forward and reverse primer at 100 μM to amplify across the entire aminoacyl-tRNA synthetase (aaRS) gene, as well as 158 μL sterile water to bring the total volume to 50 μL. Reactions were run on the thermal cycler at 95° C. for 180 s followed by 30 cycles of 95° C. for 45 s, 55° C. for 30 s, 72° C. for 135 s. Once cycles were completed, samples underwent a 10 min 72° C. final extension and hold at 4° C. until they were removed from the thermal cycler.

Digested pRS315-KanRmod vectors with tRNA_{CUA}^{Tyr} (tyrosyl transfer RNA with a CUA anticodon) were prepared in the same manner as for the E. coli Tyrosyl-tRNA synthetase (TyrRS) saturation mutagenesis library (see above). For each of the DOPARS error-prone polymerase chain reaction (epPCR) libraries at both concentrations of mutagenic dNTPs, the following masses of DNA were Pellet Painted to concentrate: 4 μg error-prone polymerase chain reaction (epPCR)-amplified aminoacyl-tRNA synthetase (aaRS), 1 μg double digested pRS315-KanRmod vector, and 1 μg pCTCON2-FAPB2.3.6L1TAG. Preparation of electrocompetent cells, electroporations, and subsequent characterization proceeded in same manner as construction of E. coli Tyrosyl-tRNA synthetase (TyrRS) and Leucyl tRNA synthetase (LeuRS) libraries.

### Yeast Transformations, Propagation, and Induction

Reporter plasmids pCTCON2-FAPB2.3.6L1TAG or pCTCON2-FAPB2.3.6 (TRP marker) and suppression machinery plasmids (LEU marker) were co-transformed into Zymo competent RJY100 cells, plated on solid SD-SCAA media (-TRP-LEU-URA), and grown at 30° C. until colonies appeared (3 days). WT controls containing only pCTCON2-FAPB2.3.6 were transformed similarly into Zymo competent RJY100 cells, plated on solid SD-SCAA media (-TRP-URA), and grown at 30° C. until colonies appeared (3 days). Inoculation and propagation in liquid SD media and induction in SG media are described in Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269. Briefly: Three separate transformant colonies (biological triplicates) were inoculated from each plate except the wild type (WT) control, where only one colony was inoculated, in 5 mL SD media of the same composition as the plates from transformations. All liquid cultures were supplemented with penicillin-streptomycin to prevent bacterial contamination. Liquid cultures were grown to saturation and then diluted to OD₆₀₀ 1 in 5 mL of the same media. The diluted cultures were grown to OD₆₀₀ 2-5 (usually 4-6 h at 30° C. with shaking) and then induced in 2 mL SG media at OD₆₀₀ 1. Induction cultures with no noncanonical amino acid (ncAA) and 1 mM of each respective noncanonical amino acid (ncAA) were prepared for each replicate. The WT control was only induced with no noncanonical amino acid (ncAA). In the case of the error-prone polymerase chain reaction (epPCR) aminoacyl-tRNA synthetases (aaRSs), induction cultures with 0.1 mM of the respective noncanonical amino acid (ncAA) were also prepared. Induced cultures were incubated at 20° C. with shaking at 300 rpm for 16 h.

For library propagation and induction, the steps were identical but in 100 mL media inoculated from a 2 mL glycerol stock of the library with propagation and inductions in 100 mL in order to preserve the full diversity of the library.

### Flow Cytometry Data Collection and Analysis

Freshly induced samples were labeled in 1.7 mL microcentrifuge tubes or 96-well V-bottom plates. Flow cytometry was performed on an Attune NxT flow cytometer (Life Technologies) at the Tufts University Science and Technology Center. Detailed protocols describing the antibody labeling process are described in Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269. Briefly: 2 million cells were removed to either microcentrifuge tubes (controls) or 96-well V-bottom plates and centrifuged to pellet. Supernatant was aspirated or decanted and cells were resuspended in room temperature phosphate-buffered saline (PBSA) to wash. Centrifugation, aspiration/decanting, and wash steps were repeated twice more. Samples were resuspended in 50 μL room temperature phosphate-buffered saline (PBSA) with 1:500 dilutions of each primary antibody label (Table 27) and incubated at room temperature on a rotary wheel or orbital shaker for 30 min. Following primary labeling, samples were kept on ice or in a refrigerated centrifuge at 4° C. for remainder of steps, until resuspension for evaluation on the flow cytometer. After 30 min primary labeling, cells were resuspended in ice-cold phosphate-buffered saline (PBSA), pelleted, and aspirated/decanted. Wash steps were repeated twice more to remove extraneous primary label. Samples were resuspended in 50 μL ice-cold phosphate-buffered saline (PBSA) with 1:500 dilutions of each secondary label (Table 27) and incubated on ice in the dark for 15 min. Samples were diluted in ice-cold phosphate-buffered saline (PBSA), pelleted, and aspirated/decanted. Wash steps were repeated once more, and cells were either immediately resuspended for evaluation on the flow cytometer or kept as wet pellets on ice or at 4° C. in the dark until resuspension (up to 6 h).

TABLE 27
Antibody labels and dilutions used in preparation for detection
of yeast-displayed constructs via flow cytometry.

| Detection | Primary label (dilution) | Secondary label (dilution) |
|---|---|---|
| HA | Mouse anti-HA (1:500) | Goat anti-mouse Alexa |
| tag (displaying) | | Fluor 488 (1:500) |
| c-Myc tag (full | Chicken anti- | Goat anti-chicken Alexa |
| length) | CMYC (1:500) | Fluor 647 (1:500) |

### Calculating Relative Readthrough Efficiency (RRE) and Maximum Misincorporation Frequency (MMF)

Flow cytometry data analysis was performed using FlowJo and Microsoft Excel. Detailed descriptions of the calculations for relative readthrough efficiency (RRE) and maximum misincorporation frequency (MMF) with corresponding error propagation are provided in Potts, K. A., Stieglitz, J. T., Lei, M., Van Deventer, J. A. (2020) Reporter system architecture affects measurements of noncanonical amino acid incorporation efficiency and fidelity, Mol. Syst. Des. Eng. 5, 573-588 and in Stieglitz, J. T., Kehoe, H. P., Lei, M., and Van Deventer, J. A. (2018) A Robust and Quantitative Reporter System To Evaluate Noncanonical Amino Acid Incorporation in Yeast, ACS Synth Biol 7, 2256-2269.

### Fluorescence-Activated Cell Sorting (FACS)

Aminoacyl-tRNA synthetase library populations were induced and labeled using the methods described above. For naïve (unsorted) library screens, a larger number of cells were used for antibody labeling and antibody/PBSA volumes for primary and secondary labeling were adjusted accordingly. A number of cells was used that was at minimum ten times larger than the library population being sorted for all subsequent screens. Cell pellets were resuspended in ice-cold phosphate-buffered saline (PBSA) immediately prior to sorting. Samples were sorted using a FACSAria™ III (Becton, Dickinson and Company) flow cytometer or a combination of a MoFlow Legacy (Beckman Coulter) and a FACSAria™ III at the Tufts University Flow Cytometry Core. Sorted samples were collected in 14 mL culture tubes containing 1 mL SD-SCAA (-TRP-LEU-URA) supplemented with penicillin-streptomycin. Following sorting, the sides of the culture tubes were washed with an additional 1 mL SD-SCAA (-TRP-LEU-URA), then transported back to the main laboratory facilities. An additional 3 mL SD-SCAA (-TRP-LEU-URA) was added to each sample and cultures were then grown at 30° C. with shaking at 300 rpm until saturated (2-3 days). Subsequent flow cytometry characterization was performed on each sorted population before the following round of screening (see above for details).

Aminoacyl-tRNA Synthetase (aaRS) Characterization Post-FACS

Once library populations with low cAA and high noncanonical amino acid (ncAA) were isolated, aminoacyl-tRNA synthetase (aaRS) plasmid DNA was purified using a Zymoprep Yeast Plasmid Miniprep II kit with slightly modified manufacturer's protocols. 500 μL of 5 mL cultures library populations that had been previously propagated for flow cytometry characterization were diluted into 4.5 mL of the same media, supplemented with penicillin-streptomycin. Cultures were grown for 4 h at 30° C. with shaking at 300 rpm. 1 mL of each culture was removed to a microcentrifuge tube and pelleted at 13,000 rpm for 30 s. Supernatant was aspirated and each pellet was resuspended in 200 μL Solution I with 6 μL reconstituted zymolase from the Zymo kit. Each sample was vortexed briefly and then incubated at 37° C. with shaking at 300 rpm overnight or up to 24 h. 200 μL of Solution II from the Zymo kit was added and tubes were inverted to mix. 400 μL of Solution III was added and tubes were inverted to mix. Samples were pelleted at 15,000 rpm (max speed) for 30 min to separate out cell debris. The supernatant was transferred to Epoch Life Science E. coli DNA purification columns and purified using Epoch protocols. DNA was eluted in 40 μL sterile water and then transformed into chemically competent E. coli DH5alphaZ1 cells and plated on LB media with 34 μg/mL kanamycin. 10-12 individual colonies were inoculated into separate 5 mL LB cultures with 50 μg/mL kanamycin and grown overnight at 37° C. with shaking at 300 rpm. Cultures were miniprepped using an Epoch E. coli GenCatch™ Plasmid DNA Mini-Prep Kit and submitted for sequencing.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A Tyrosyl-tRNA Synthetase (TyrRS) polypeptide, or a functional fragment thereof, having at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and comprising an alteration at an amino acid position selected from the group consisting of Y37, L71, V72, Q179, D182, and Q195 and having tRNA synthetase activity.

2. The Tyrosyl-tRNA Synthetase (TyrRS) polypeptide of claim 1, further comprising an alteration at an amino acid position wherein:
i) Y37 is Y37A, Y37D, Y37E, Y37G, Y37H, Y37I, Y37L, Y37M, Y37Q, Y37T, or Y37V;
ii) L71 is L71V, L71D, L71I, L71M, L71R, L71T, or L71V;
iii) V72 is V72A, V72H, V72I, V72L, V72M, V72Q, V72R, V72S, or V72T;
iv) Q179 is Q179A, Q179D, Q179E, Q179G, Q179H, Q179L, Q179M, Q179N, Q179P, Q179S, Q179T, or Q179V;
v) D182 is D182G, or D182S;
vi) F183 is F183A, F183D, F183G, F183H, F183I, F183L, F183N, F183P, F183Q, F183R, F183T, or F183V;
vii) L186 is L186E, L186S, or L186V; and
viii) Q195 is Q195A, Q195E, Q195G, Q195H, Q195I, Q195K, Q195L, Q195M, Q195S, Q195T, or Q195V.

3. The Tyrosyl-tRNA Synthetase (TyrRS) polypeptide of claim 1, wherein the TyrRS polypeptide comprises a combination of alterations selected from (1) Y37L and L71V, (2) Y37G, L71T, and D182S, (3) L71V and L186A, (4) Y37V and L71V, (5) Y37L, L71V, and L186A, and (6) L71I, V72V, Q179Q, D182G, F183M, L186A, and Q195Q.

4. A Leucyl-tRNA Synthetase (LeuRS) polypeptide, or a functional fragment thereof, having at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and comprising an alteration at an amino acid position selected from the group consisting of M40, L41, S496, Y499 and Y527 and having tRNA synthetase activity.

5. The Leucyl-tRNA Synthetase (LeuRS) polypeptide of claim 4, wherein the alteration is selected at an amino acid position selected from the following:
i) M40 is M40A, M40G, M40L, M40P, M40Q, or M40S;
ii) L41 is L41A, L41E, L41G, L41H, L41N, L41P, L41T, or L41V;
iii) S496 is S496A, S496G, or S496T;
iv) Y499 is Y499A, Y499C, Y499F, Y499G, Y499H, Y499I, Y499L, Y499N, Y499S, Y499T, or Y499V;
v) Y527 is Y527C, Y527D, Y527F, Y527G, Y527H, Y527I, Y527N, Y527R, Y527S, Y527T, or Y527V;
vi) H537 is H537A, H537C, H537F, H537L, or H537S.

6. The Leucyl-tRNA Synthetase (LeuRS) polypeptide of claim 4, wherein the LeuRS polypeptide comprises a combination of alterations selected from (1) M40G and S496T, (2) S496T and H537G, (3) S496G and H537G, (4) L41P, S496G, and H537G, (5) M40P, S496G, H537G, (6) L41G and H537G, (7) M40A and H537G, and (8) M40G, L41P, S496GY499A, Y527C, and H537G.

7. The polypeptide of claim 1, wherein the polypeptide has aminoacylation activity specific for one or more noncanonical amino acid.

8. The polypeptide of claim 7, wherein the noncanonical amino acid(s) is selected from one or more of O-methyl-L-tyrosine (OmeY); p-acetyl-L-phenylalanine (AcF); p-azido-L-phenylalanine (AzF); p-propargyloxy-L-phenylalanine (OPG); 4-azidomethyl-L-phenylalanine (AzMF); 4-borono-L-phenylalanine (BPhe); 3,4-dihydroxy-L-phenylalanine (DOPA); O-(2-Bromoethyl)-tyrosine (Obey); 4-iodo-L-phenylalanine (IPhe); L-α-aminocaprylic acid (AC); NE-azido-L-lysine (AzK); 3-Amino-L-tyrosine (ATyr); 4-Amino-L-phenylalanine (APhe); dimethyl-L-lysine (DMK); Boc-L-lysine (BocK); (S)-2-amino-6-((2-azidoethoxy)carbonylamino)hexanoic acid (LysN3); and 2-Amino-6-(prop-2-ynoxycarbonylamino)hexanoic acid (LysAlk), O-(2-Bromoethyl)-L-tyrosine, O-Sulfo-L-tyrosine (SY), 2-amino-3-[4-(carboxymethyl) phenyl]propanoic acid (CMF), L-p-hydroxy-phenyllactic acid (Ester), L-2-Amino-4-phosphonobutyric acid (PSA), O-phospho-L-serine (OPS), Acetyl-L-lysine (AcK), 4-benzoyl-1-phenylalanine (Bpa), and N⁶-((2-(3-methyl-3H-diazirin-3-yl)ethoxy)carbonyl)-L-lysine (Photo-Lysine, Phk).

9. A polynucleotide encoding the polypeptide of claim 1.

10. An expression vector comprising the polynucleotide of claim 9.

11. A cell comprising the expression vector of claim 10.

12. An in vitro method of producing a protein comprising a noncanonical amino acid, the method comprising:
(a) contacting an in vitro translation system or cell with a Tyrosyl-tRNA Synthetase (TyrRS) polypeptide, or a functional fragment thereof, having at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and comprising an alteration at an amino acid position selected from the group consisting of Y37, L71, V72, Q179, D182, and Q195 and having tRNA synthetase activity,
(b) contacting the in vitro translation system or cell of (a) with one or more noncanonical amino acids or a composition thereof and a polynucleotide encoding a protein comprising one or more noncanonical amino acids; and
(c) expressing the protein comprising one or more noncanonical amino acids using the in vitro translation system or cell.

13. An in vitro method of producing a protein comprising a noncanonical amino acid, the method comprising:
(a) contacting an in vitro translation system or cell with an expression vector encoding a Leucyl-tRNA Synthetase (LeuRS) polypeptide, or a functional fragment thereof, having at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and comprising an alteration at an amino acid position selected from the group consisting of M40, L41, 5496, Y499 and Y527 and having tRNA synthetase activity, and expressing the polypeptide(s) in the cell;
(b) contacting the in vitro translation system or cell of (a) with one or more noncanonical amino acids or a composition thereof and producing a protein comprising one or more noncanonical amino acids in the cell; and
(c) expressing said protein comprising one or more noncanonical amino acids using the in vitro translation system or cell.

14. The method of claim 13, wherein the cell is genetically engineered or mutated to utilize noncanonical amino acids.

15. A system for producing and selecting for a protein comprising a noncanonical amino acid, the system comprising: an in vitro translation system or cell comprising a Tyrosyl-tRNA Synthetase (TyrRS) polypeptide, or a functional fragment thereof, having at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and comprising an alteration at an amino acid position selected from the group consisting of Y37, L71, V72, Q179, D182, and Q195 and having tRNA synthetase activity, wherein the in vitro translation system or cell comprises one or more noncanonical amino acids or a composition thereof, and wherein a protein comprising one or more noncanonical amino acids is produced in the in vitro translation system or cell.

16. A system for producing and selecting for a protein comprising a noncanonical amino acid, the system comprising: an in vitro translation system or cell containing a Leucyl-tRNA Synthetase (LeuRS) polypeptide, or a functional fragment thereof, having at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and comprising an alteration at an amino acid position selected from the group consisting of M40, L41, S496, Y499 and Y527 and having tRNA synthetase activity, wherein the in vitro translation system or cell comprises one or more noncanonical amino acids or a composition thereof, and wherein a protein comprising one or more noncanonical amino acids is produced in the in vitro translation system or cell.

17. A protein comprising one or more noncanonical amino acids, the protein being produced, generated or selected by the method of claim 13.

18. A method of controlling the replication of a cell or virus, the method comprising:
(a) (i) altering a gene encoding a polypeptide in the cell or a virus infecting the cell to encode the polypeptide altered to comprise a noncanonical amino acid and/or (ii) knocking out the gene in the cell or the virus infecting the cell, and contacting the cell with a polynucleotide sequence encoding the polypeptide altered to comprise the noncanonical amino acid, such that replication of the cell or virus is reduced or eliminated in the absence of expression of the polypeptide containing the noncanonical amino acid,
(b) contacting the cell with an expression vector encoding a Tyrosyl-tRNA Synthetase (TyrRS) polypeptide, or a functional fragment thereof wherein the polypeptide has at least about 85% amino acid sequence identity to the following polypeptide sequence: MASSNLIKQLQERGLVAQVTDEEALAERLAQGPIALYCGFDPTADSLHLGHLVPLLCLK RFQQAGHKPVALVGGATGLIGDPSFKAAERKLNTEETVQEWVDKIRKQVAPFLDFDCG ENSAIAANNYDWFGNMNVLTFLRDIGKHFSVNQMINKEAVKQRLNREDQGISFTEFSYN LLQGYDFACLNKQYGVVLQIGGSDQWGNITSGIDLTRRLHQNQVFGLTVPLITKADGTK FGKTEGGAVWLDPKKTSPYKFYQFWINTADADVYRFLKFFTFMSIEEINALEEEDKNSG KAPRAQYVLAEQVTRLVHGEEGLQAAKRITECLFSGSLSALSEADFEQLAQDGVPMVE MEKGADLMQALVDSELQPSRGQARKTIASNAITINGEKQSDPEYFFKEEDRLFGRFTLLR RGKKNYCLICWK (SEQ ID NO: 1), and comprising an alteration at an amino acid position selected from the group consisting of Y37, L71, V72, Q179, D182, and Q195 and having tRNA synthetase activity, and expressing the polypeptide(s) in the cell;
(c) controlling replication of the cell and/or virus by contacting the cell of (a) with the noncanonical amino acid, wherein replication of the cell and/or virus is reduced or eliminated in the absence of the noncanonical amino acid.

19. A method of controlling the replication of a cell or virus, the method comprising:
(a) (i) altering a gene encoding a polypeptide in the cell or a virus infecting the cell to encode the polypeptide altered to comprise a noncanonical amino acid and/or (ii) knocking out the gene in the cell or the virus infecting the cell, and contacting the cell with a polynucleotide sequence encoding the polypeptide altered to comprise the noncanonical amino acid, such that replication of the cell or virus is reduced or eliminated in the absence of expression of the polypeptide containing the noncanonical amino acid,
(b) contacting the cell an expression vector encoding a Leucyl-tRNA Synthetase (LeuRS) polypeptide, or a functional fragment thereof, wherein the polypeptide has at least about 85% amino acid sequence identity to the following polypeptide sequence: MQEQYRPEEIESKVQLHWDEKRTFEVTEDESKEKYYCLSMLPYPSGRLHMGHVRNYTI GDVIARYQRMLGKNVLQPIGWDAFGLPAEGAAVKNNTAPAPWTYDNIAYMKNQLKM LGFGYDWSRELATCTPEYYRWEQKFFTELYKKGLVYKKTSAVNWCPNDQTVLANEQV IDGCCWRCDTKVERKEIPQWFIKITAYADELLNDLDKLDHWPDTVKTMQRNWIGRSEG VEITFNVNDYDNTLTVYTTRPDTFMGCTYLAVAAGHPLAQKAAENNPELAAFIDECRN TKVAEAEMATMEKKGVDTGFKAVHPLTGEEIPVWAANFVLMEYGTGAVMAVPGHDQ RDYEFASKYGLNIKPVILAADGSEPDLSQQALTEKGVLFNSGEFNGLDHEAAFNAIADK LTAMGVGERKVNYRLRDWGVSRQRYWGAPIPMVTLEDGTVMPTPDDQLPVILPEDVV MDGITSPIKADPEWAKTTVNGMPALRETDTFDTFMESSWYYARYTCPQYKEGMLDSEA ANYWLPVDIYIGGIEHAIMHLLYFRFFHKLMRDAGMVNSDEPAKQLLCQGMVLADAFY YVGENGERNWVSPVDAIVERDEKGRIVKAKDAAGHELVYTGMSKMSKSKNNGIDPQV MVERYGADTVRLFMMFASPADMTLEWQESGVEGANRFLKRVWKLVYEHTAKGDVA ALNVDALTENQKALRRDVHKTIAKVTDDIGRRQTFNTAIAAIMELMNKLAKAPTDGEQ DRALMQEALLAVVRMLNPFTPHICFTLWQELKGEGDIDNAPWPVADEKAMVEDSTLV VVQVNGKVRAKITVPVDATEEQVRERAGQEHLVAKYLDGVTVRKVIYVPGKLLNLVV G (SEQ ID NO: 2), and comprising an alteration at an amino acid position selected from the group consisting of M40, L41, S496, Y499 and Y527 and having tRNA synthetase activity, and expressing the polypeptide(s) in the cell;
(c) controlling replication of the cell and/or virus by contacting the cell of (a) with the noncanonical amino acid, wherein replication of the cell and/or virus is reduced or eliminated in the absence of the noncanonical amino acid.

20. The method of claim 19, wherein the cell is a mammalian cell, yeast cell, bacterial cell, plant cell, or insect cell.
